(19) United States
(12) Patent Application Publication
(10) Pub. No.: US 2026/0076557 A1
(43) Pub. Date: Mar. 19, 2026

(54) METHODS AND SYSTEMS FOR VIRTUAL REALITY COLOR WAVELENGTH SENSITIVITY TESTING

(71) Applicant: Zenni Optical, Inc., Novato, CA (US)

(72) Inventors: Steven LEE, Barrington, IL (US); Julia ZHEN, Novato, CA (US); ChyrSong TING, Novato, CA (US); Matthew James GOLINO, Brookhaven, GA (US); Justin Paul DEMPSEY, Ottawa (CA); Jeffrey Joseph FILLINGHAM, Dartmouth (CA)

(21) Appl. No.: 18/885,511

(22) Filed: Sep. 13, 2024

(51) Int. Cl. A61B 3/06; A61B 3/00

(52) U.S. Cl. CPC A61B 3/066; A61B 3/005

(57) ABSTRACT

A virtual reality (VR) system can be implemented for evaluating color wavelength sensitivity. The system can use an electronic device featuring a head-mounted display (HMD) and eye-tracking sensors. The system can generate a VR user interface that creates a three-dimensional virtual environment, rendered on the HMD. Within this virtual space, the system can simulate a variety of color wavelength tasks. As users engage with these tasks, the eye-tracking sensors can continuously monitor responses to the simulated tasks. The system can then analyze the tracked data for color wavelength sensitivity performance.

### TECHNICAL FIELD

The present inventions relate to vision test technology. More specifically, methods, systems, devices, and non-statutory computer-readable storage media are applied to implement vision testing in an extended reality environment for evaluating color wavelength sensitivity.

### BACKGROUND

Traditional visual assessment methods have been the cornerstone of evaluating eye health and vision for many years. These methods are typically conducted in clinical environments, where specialized equipment and standardized procedures are used to ensure accurate and reliable results. The parameters for these assessments are generally fixed, reflecting the controlled nature of the clinical setting.

Over time, these techniques have become the accepted standard for diagnosing and monitoring visual conditions, forming the basis of routine eye care practices in medical offices, hospitals, and specialized eye care facilities. Despite their widespread use, these methods have traditionally been limited to professional settings, where they can be conducted under the supervision of trained healthcare providers using dedicated equipment.

### SUMMARY

The present disclosure relates to innovative methods and systems that can revolutionize vision care, making vision testing and other exams more accessible and affordable for patients. Additionally, it is contemplated that the principles and features of the present disclosure can be implemented in numerous other applications of display technology, including headsets, heads-up displays, and other micro-displays (e.g., microLED and microOLED) to address challenges and limitations inherent in such products and their uses.

In accordance with at least some embodiments disclosed herein is the realization that traditional methods for visual assessment do not allow for dynamic adjustment of test parameters, leading to less accurate assessments, nor can they be implemented to test eyes and vision at home using household devices in a consistent and environment-locked manner.

Some embodiments are directed to a method of implementing a virtual vision test at an electronic device including a head-mounted display (HMD) and a camera. The method includes executing a user application configured to enable the virtual vision test; generating a virtual reality (VR) user interface corresponding to a three-dimensional (3D) virtual environment; focusing the camera on an eye area of a user wearing the electronic device; displaying, on the user interface, a visual stimulus corresponding to the virtual vision test; while displaying the visual stimulus, in real time, capturing a sequence of eye images using the camera of the electronic device; determining eye movement information including a temporal sequence of eyeball positions based on the sequence of eye images; and comparing the visual stimulus and the eye movement information to determine an eye health condition.

In some embodiments, a user application can be implemented by a head-mounted display configured to create a customized extended reality (XR) environment for a user engaged on an XR information platform. Products may be rendered for the user in a three-dimension format in the XR environment, thereby facilitating eyewear selection and fitting. The XR can be an umbrella term encapsulating Augmented Reality (AR), Virtual Reality (VR), Mixed Reality (MR), and everything in between. In this application, any embodiments that apply a VR system can be implemented using an AR or MR system as well.

Some embodiments are directed to a method of implementing a virtual reality (VR) system for testing light sensitivity and prescribing customized LCD tinted lenses. The method is performed at an electronic device including a head-mounted display and eye-tracking sensors. The method includes generating a VR user interface corresponding to a three-dimensional virtual environment and rendering the VR user interface on the head-mounted display. The method also includes simulating various lighting conditions sequentially in the VR user interface. While simulating the various lighting conditions, in real time, the method continuously tracks, using the eye-tracking sensors, gaze direction, blink rate, squinting, and pupillary responses to the simulated lighting conditions. The method also includes evaluating the tracked data for light sensitivity performance. In this way, the method enables comprehensive assessment of an individual's light sensitivity in a controlled, immersive environment, facilitating the prescription of customized LCD tinted lenses tailored to the user's specific visual needs.

Some embodiments are directed to a method of implementing a virtual reality (VR) system for recommending lens tints through an interactive vision sensitivity test. The method is performed at an electronic device including a head-mounted display (HMD) and eye-tracking sensors. The method includes generating a VR user interface corresponding to a three-dimensional virtual environment and rendering the VR user interface on the head-mounted display. The method also includes simulating various lighting conditions and glare levels sequentially in the VR user interface. While simulating the various lighting conditions and glare levels, in real time, the method continuously tracks, using the eye-tracking sensors, user responses to the simulated lighting conditions and glare levels. The method also includes evaluating the tracked data for vision sensitivity performance. In this way, the method enables a comprehensive and interactive assessment of a user's vision sensitivity under various lighting and glare conditions in a controlled, immersive environment, facilitating the recommendation of personalized lens tints based on the user's specific visual responses and needs.

Some embodiments are directed to a method of implementing a virtual reality (VR) system for evaluating color perception. The method is performed at an electronic device including a head-mounted display (HMD) and eye-tracking sensors. The method includes generating a VR user interface corresponding to a three-dimensional virtual environment and rendering the VR user interface on the head-mounted display. The method also includes simulating various color-coded challenges and puzzles under varying luminosities and backgrounds in the VR user interface. While simulating the color-coded challenges and puzzles, in real time, the method continuously tracks, using the eye-tracking sensors, user responses to the simulated challenges and puzzles. The method also includes evaluating the tracked data for color perception performance. In this way, the method enables a comprehensive and dynamic assessment of color perception abilities under diverse visual conditions in an immersive, controlled environment. By utilizing interactive challenges and puzzles, the system can evaluate nuanced aspects of color perception, potentially uncovering subtle deficiencies or strengths that might not be apparent in traditional color vision tests.

Some embodiments are directed to a method of implementing a virtual reality (VR) system for evaluating color perception. The method is performed at an electronic device including a head-mounted display (HMD) and eye-tracking sensors. The method includes generating a VR user interface corresponding to a three-dimensional virtual environment and rendering the VR user interface on the head-mounted display. The method also includes simulating various color perception tasks under varying luminosities and backgrounds in the VR user interface. While simulating the color perception tasks, in real time, the method continuously tracks, using the eye-tracking sensors, user responses to the simulated tasks. The method also includes evaluating the tracked data for color perception performance. In this way, the method enables a comprehensive and dynamic assessment of color perception abilities under diverse visual conditions in an immersive, controlled environment. By utilizing a range of color perception tasks and varying environmental factors, the system can evaluate, for example, nuanced aspects of color vision, potentially uncovering subtle deficiencies or strengths that might not be apparent in traditional color vision tests.

Some embodiments are directed to a method of implementing a virtual reality (VR) system for evaluating color perception, with a specific focus on color wavelength sensitivity. The method is performed at an electronic device including a head-mounted display (HMD) and eye-tracking sensors. The method includes generating a VR user interface corresponding to a three-dimensional virtual environment and rendering the VR user interface on the head-mounted display. The method also includes simulating various color wavelength tasks in the VR user interface. While simulating the color wavelength tasks, in real time, the method continuously tracks, using the eye-tracking sensors, user responses to the simulated tasks. The method also includes evaluating the tracked data for color wavelength sensitivity performance. In this way, the method enables a precise and comprehensive assessment of an individual's sensitivity to specific color wavelengths in an immersive, controlled environment. By utilizing specialized color wavelength tasks and advanced eye-tracking technology, the system can evaluate nuanced aspects of color perception at the wavelength level, potentially uncovering subtle variations in color sensitivity that might not be detected by conventional color vision tests.

Some embodiments are directed to a method of implementing a virtual reality (VR) system for testing and recommending adaptive eyewear for color blindness. The method is performed at an electronic device including a head-mounted display (HMD) and eye-tracking sensors. The method includes generating a VR user interface corresponding to a three-dimensional virtual environment and rendering the VR user interface on the head-mounted display. The method also includes simulating various real-world scenarios in the VR user interface. While simulating the real-world scenarios, in real time, the method continuously tracks, using the eye-tracking sensors, user responses to the simulated scenarios. The method also includes evaluating the tracked data for color perception performance. In this way, the method enables a comprehensive and realistic assessment of color vision deficiencies in simulated everyday situations, providing a basis for recommending personalized adaptive eyewear. By utilizing a range of real-world scenarios and advanced eye-tracking technology, the system can evaluate the effectiveness of different adaptive eyewear options in improving color perception for individuals with color blindness.

Some embodiments are directed to a system for implementing a virtual eye test. The system includes a head-mounted display including a display and one or more cameras. The system also includes one or more processors and memory storing one or more programs configured to be executed by the one or more processors. The one or more programs includes instructions for a user interface module configured to generate a virtual reality (VR) user interface corresponding to a three-dimensional virtual environment. The one or more programs also includes instructions for a rendering module configured to render the VR user interface on the HMD. The one or more programs also includes instructions for a simulation module configured to simulate one or more scenarios in the VR user interface. The one or more programs also includes instructions for a tracking module configured to continuously track, using at least one of the one or more cameras and/or eye-tracking sensors, eye movements and/or responses to visual stimuli presented in the one or more scenarios. The one or more programs also includes instructions for an evaluation module configured to analyze user interactions and system performance to determine and/or measure at least one of: light sensitivity performance, vision sensitivity performance, color sensitivity performance, color perception performance, and/or color wavelength sensitivity performance.

In some embodiments, a non-transitory computer readable storage medium is provided that can store one or more programs for execution by one or more processors of a computer system, the one or more programs including instructions for performing any of the methods described herein.

In some embodiments, an electronic device is provided that can comprise an HMD, a camera and/or eye-tracking sensors, one or more processors, and memory for storing one or more programs for execution by the one or more processors, the one or more programs including instructions for performing any of the methods described herein.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE FIGURES

Various features of illustrative embodiments of the inventions are described below with reference to the drawings. The illustrated embodiments are intended to illustrate, but not to limit, the inventions.

FIG. 1 is an example data processing environment having one or more servers communicatively coupled to one or more computer devices (e.g., includes a headset device), in accordance with some embodiments.

FIG. 2 is an environment in which a computer device (e.g., a headset device) is applied to facilitate visual assessment or eyewear fitting, in accordance with some embodiments.

FIG. 3 is a block diagram of a computer system (e.g., including a headset device) configured to implement vision assessment or eyewear fitting, in accordance with some embodiments.

FIG. 4 is a block diagram of a machine learning system for training and applying machine learning models (e.g., for glass making), in accordance with some embodiments.

FIG. 5A is a structural diagram of an example neural network applied to process input data in a machine learning model, in accordance with some embodiments, and FIG. 5B is an example node in the neural network, in accordance with some embodiments.

FIG. 6A is an example “tumbling E” chart applied in a visual acuity test, and FIGS. 6B-6E are example patterns applied in an astigmatism test, a stereopsis test, a visual field test, and a color blindness test, in accordance with some embodiments.

FIG. 7 is another example visual pattern applied to test visual acuity and astigmatism, in accordance with some embodiments.

FIGS. 8A-8D include four diagrams of example graphical user interfaces rendered to determine a visual acuity score in a virtual environment created by a headset device, in accordance with some embodiments.

FIGS. 9A-9C include three diagrams of example graphical user interfaces rendered to determine a nearsighted or farsighted power in a virtual environment created by a headset device, in accordance with some embodiments.

FIGS. 10A-10F include six diagrams of example graphical user interfaces rendered to determine eye stigmatism in a virtual environment created by a headset device, in accordance with some embodiments.

FIGS. 11A and 11B are diagrams showing an example vision test system, in accordance with some embodiments.

FIGS. 12A-12F show a flow diagram of an example process for implementing a virtual reality (VR) system for testing light sensitivity and prescribing customized LCD tinted lenses, according to some embodiments.

FIGS. 13A-13F show a flow diagram of an example process for recommending lens tints through an interactive vision sensitivity test, according to some embodiments.

FIGS. 14A-14F show a flow diagram of an example process for implementing a virtual eye test for color blindness using color-coded challenges and/or puzzles, according to some embodiments.

FIGS. 15A-15F show a flow diagram of an example process for implementing a virtual eye test for evaluating color perception under varying luminosities and backgrounds, according to some embodiments.

FIGS. 16A-16F show a flow diagram of an example process for testing sensitivity to specific color wavelengths for specialized eyewear prescriptions, according to some embodiments.

FIGS. 17A-17F show a flow diagram of an example process for testing and/or recommending adaptive eyewear for color blindness in real-world simulations, according to some embodiments.

FIG. 18 is a schematic diagram showing an example vision test, in accordance with some embodiments.

FIG. 19A shows illustrations of example visual scenarios for VR light sensitivity testing and LCD tinted lens prescription system, according to some embodiments.

FIG. 19B is a block diagram of example components for a VR light sensitivity testing and LCD tinted lens prescription system, according to some embodiments.

FIG. 20A shows illustrations of example photorealistic view glare-prone environments for VR lens tint recommendation through interactive vision sensitivity test, according to some embodiments.

FIG. 20B is a block diagram of example components for VR lens tint recommendation through interactive vision sensitivity test, according to some embodiments.

FIG. 21A shows illustrations of example 3D virtual environments for VR-enabled color blindness test using color-coded challenges and puzzles, according to some embodiments.

FIG. 21B is a block diagram of example components for VR-enabled color blindness test using color-coded challenges and puzzles, according to some embodiments.

FIG. 22A shows illustrations of example backgrounds for VR-based color perception evaluation system, according to some embodiments.

FIG. 22B is a block diagram of example components for VR-based color perception evaluation system, according to some embodiments.

FIG. 23A shows illustrations of example color presentations for VR-based color wavelength sensitivity evaluation system, according to some embodiments.

FIG. 23B is a block diagram of example components for VR-based color wavelength sensitivity evaluation system, according to some embodiments.

FIG. 24A shows illustrations of example real-world scenarios for VR-based adaptive eyewear recommendation system for color blindness, according to some embodiments.

FIG. 24B is a block diagram of example components for VR-based adaptive eyewear recommendation system for color blindness, according to some embodiments.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

Moreover, various aspects of the present disclosure can be implemented in combination with aspects of other virtual-reality technology developed by the present applicant, for example, in copending U.S. Patent App. Nos. 63/560,623 (137034-5002), filed on Mar. 1, 2024, 63/569,095 (137034-5005), filed on Mar. 23, 2024, 63/642,571 (137034-5007), filed on May 3, 2024, 63/642,583 (137034-5009), filed on May 3, 2024, 63/642,593 (137034-5010), filed on May 3, 2024, 63/642,604 (137034-5011), filed on May 3, 2024, 63/644,457 (137034-5012), filed on May 8, 2024, Ser. No. 18/759,641 (137034-5018), filed on Jun. 28, 2024, Ser. No. 18/791,203 (137034-5036), filed on Jul. 31, 2024, Ser. No. 18/827,546 (137034-5050), filed Sep. 6, 2024, and Ser. No. 18/827,588 (137034-5070), filed Sep. 6, 2024, Ser. No. 18/819,311 (137034-5029), filed Aug. 29, 2024, Ser. No. 18/820,121 (137034-5047), filed Aug. 29, 2024, Ser. No. 18/820,140 (137034-5063), filed Aug. 29, 2024, App. No. TBD (137034-5105), filed Sep. 13, 2024, the entireties of each of which is incorporated herein by reference. Aspects of these copending cases can be implemented in combination with some embodiments disclosed herein, whether in addition to features thereof or as an alternative to a particular feature of an embodiment disclosed herein.

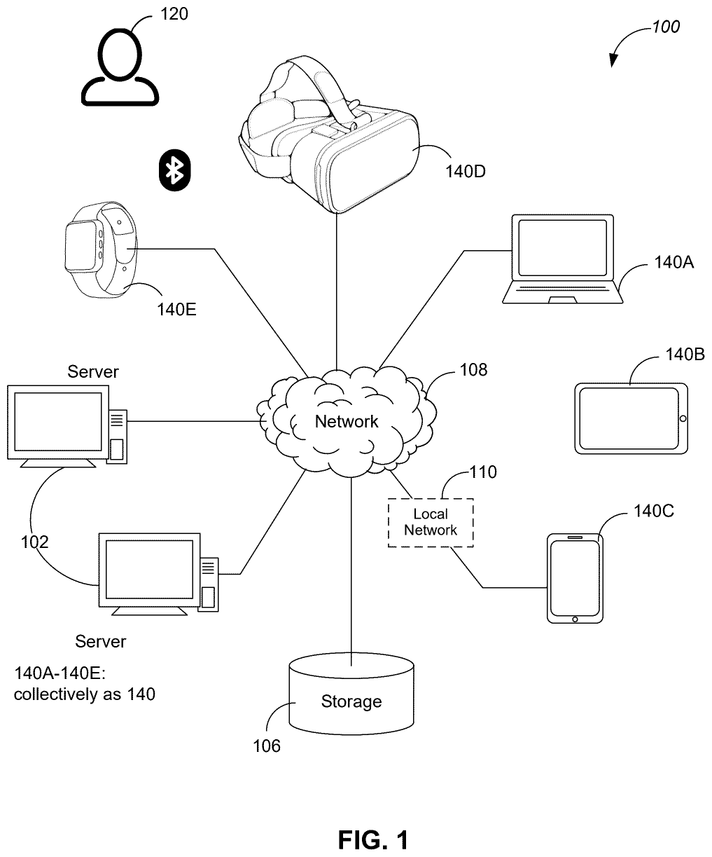

FIG. 1 is an example data processing environment 100 having one or more servers 102 communicatively coupled to one or more computer devices 140 (e.g., includes a headset device 140D), in accordance with some embodiments. The one or more computer devices 140 are electronic devices having computational capabilities, and may be, for example, desktop computers 140A, tablet computers 140B, mobile phones 140C, or intelligent, multi-sensing, network-connected home devices (e.g., a depth camera, a visible light camera). In some embodiments, the one or more computer devices 140 include a headset device 140D (also called a head-mounted display 140D) configured to render extended reality content. In some embodiments, the one or more computer devices 140 include a wireless wearable device 140E (e.g., a smart watch, a fitness band) configured to track health data (e.g., heart rate, quality of sleep) and activity data (e.g., steps walked, stairs climbed) of a user wearing the device 140E. Each computer device 140 can collect data or user inputs, executes user applications, and present outputs on its user interface. The collected data or user inputs can be processed locally at the computer device 140 and/or remotely by the server(s) 102. The one or more servers 102 provides system data (e.g., boot files, operating system images, and user applications) to the computer devices 140, and in some embodiments, processes the data and user inputs received from the computer device(s) 140 when the user applications are executed on the computer devices 140. In some embodiments, the data processing environment 100 further includes a storage 106 for storing data related to the servers 102, computer devices 140, and applications executed on the computer devices 140. For example, storage 106 may store video content, static visual content, and/or audio data.

The one or more servers 102 can enable real-time data communication with the computer devices 140 that can be remote from each other or from the one or more servers 102. Further, in some embodiments, the one or more servers 102 can implement data processing tasks that are not completed locally by the computer devices 140. For example, the computer devices 140 include a game console (e.g., the headset device 140D) that executes an interactive online gaming application. The game console receives a user instruction and sends it to a game server 102 with user data. The game server 102 generates a stream of video data based on the user instruction and user data and provides the stream of video data for display on the game console and other computer devices that can be engaged in the same game session with the game console.

The one or more servers 102, one or more computer devices 140, and storage 106 can be communicatively coupled to each other via one or more communication networks 108, which are the medium used to provide communications links between these devices and computers connected together within the data processing environment 100. The one or more communication networks 108 may include connections, such as wire, wireless communication links, or fiber optic cables. Examples of the one or more communication networks 108 include local area networks (LAN), wide area networks (WAN) such as the Internet, or a combination thereof. The one or more communication networks 108 are, optionally, implemented using any known network protocol includes various wired or wireless protocols, such as Ethernet, Universal Serial Bus (USB), FIREWIRE, Long Term Evolution (LTE), Global System for Mobile Communications (GSM), Enhanced Data GSM Environment (EDGE), code division multiple access (CDMA), time division multiple access (TDMA), Bluetooth, Wi-Fi, voice over Internet Protocol (VoIP), Wi-MAX, or any other suitable communication protocol. A connection to the one or more communication networks 108 may be established either directly (e.g., using 1G/4G connectivity to a wireless carrier), or through a network interface 110 (e.g., a router, switch, gateway, hub, or an intelligent, dedicated whole-home control node), or through any combination thereof. As such, the one or more communication networks 108 can represent the Internet of a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other electronic systems that route data and messages.

In some embodiments, the headset device 140D can be communicatively coupled to a data processing environment 100. The headset device 140D includes one or more cameras (e.g., a visible light camera, a depth camera), a microphone, a speaker, one or more inertial sensors (e.g., gyroscope, accelerometer), and a display. In some situations, the camera captures hand gestures of a user wearing the headset device 140D. In some situations, the microphone records ambient sound includes user's voice commands.

In some embodiments, the headset device 140D is communicatively coupled to one or more servers 102 and enables a centralized vision test management platform with the one or more servers 102. This vision test management platform may aggregate data (e.g., visual stimuli 338, sensor data 342, vision test results 344) from a plurality of user accounts associated with a plurality of users, analyze the aggregated data, and track vision health trends for individual users or user groups. In some embodiments, data are communicated between a headset device 140D and a server 102 in an encrypted format. In some embodiments, the vision test management platform is coupled to a global health database storing epidemiological data and configured to cross-reference the data collected from its user accounts with the epidemiological data to identify an emerging pattern and a public health concern. For example, a teenager's vision data was collected and analyzed during an extended duration of time (e.g., 10 years) to identify an individual vision development trend and cross-referenced with an average vision development trend extracted from the global health database. A doctor can rely on a cross-referencing result to determine whether the individual vision development trend is normal or whether the teenager's eyesight drops faster than average teenagers. As such, various embodiments of the vision test management platform integrate biometric data and global health analytics and provides a secure, personalized, and interactive environment for vision testing, which improves precision and user experience of vision assessments and contributes to broader public health monitoring and research initiatives.

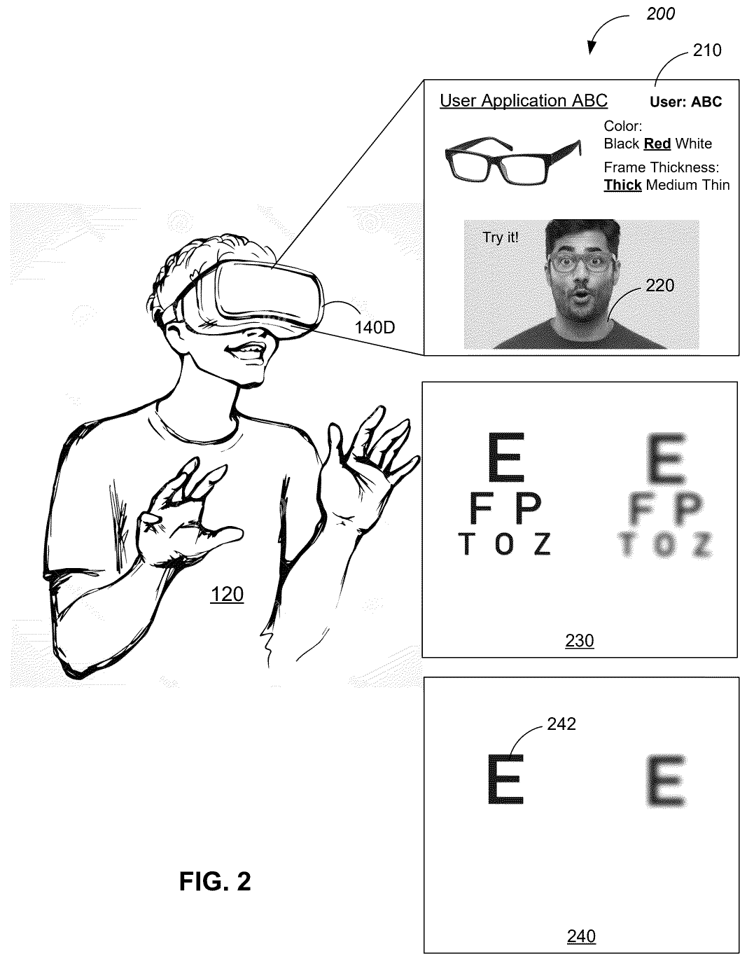

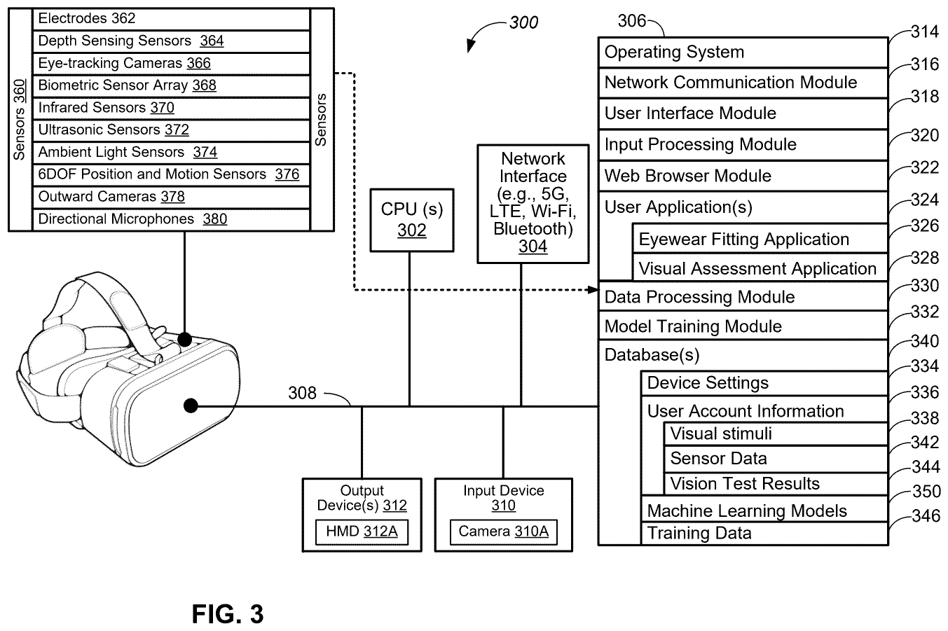

FIG. 2 is an environment 200 in which a computer device 140 (e.g., a headset device 140D) is applied to facilitate visual assessment or eyewear fitting, in accordance with some embodiments. The XR headset device 140D may be communicatively coupled within the data processing environment 100. The XR headset device 140D may include one or more cameras (e.g., a visible light camera, a depth camera), a microphone, a speaker, one or more inertial sensors (e.g., gyroscope, accelerometer), and a display. In some situations, the camera captures hand gestures of a user wearing the XR headset device 140D. In some situations, the microphone records ambient sound includes user's voice commands. The XR headset device 140D may execute a client-side eyewear fitting application 326 or a client-side visual assessment application 328 (FIG. 3) via a user account associated with a user 120 (e.g., an optometrist user, an optician user, a patient user). In some embodiments, a computer device 140 (e.g., a mobile phone 140C) distinct from the XR headset device 140D can be used to implement the client-side eyewear fitting application 326 or visual assessment application 328 (FIG. 3).

In some embodiments, a first user interface 210 can be displayed on a computer device 140 (e.g., the headset device 140D) associated with the user 120. In some embodiments, an eyewear can be tried on or displayed as being worn by a 2D or 3D image 220 of the user 120. The server 102 or computer device 140 receives, from the first user interface 210, a user feedback message indicating an issue, requesting further improvement, or confirming a fit. In some embodiments, a second user interface 230 can be displayed on a computer device 140 associated with the user 120. The second user interface 230 includes a plurality of optotypes (e.g., six optotypes E, F, P, T, O, and Z) having different sizes. In some embodiments, a third user interface 240 can be displayed on a computer device 140 associated with the user 120. The second user interface 230 can display a temporal sequence of optotypes having respective sizes. Each optotype of a corresponding size can be displayed at one time.

FIG. 3 is a block diagram of a computer system 300 (e.g., including a headset device 140D, a server, or a combination thereof) configured to implement vision assessment or eyewear fitting, in accordance with some embodiments. The computer system 300 typically, includes one or more processing units (CPUs) 302, one or more network interfaces 304, memory 306, and one or more communication buses 308 for interconnecting these components (sometimes called a chipset). The computer system 300 includes one or more input devices 310 that facilitate user input, such as a keyboard, a mouse, a voice-command input unit or microphone, a touch screen display, a touch-sensitive input pad, a gesture capturing camera, or other input buttons or controls. Furthermore, in some embodiments, the computer device 140 of the computer system 300 uses a microphone for voice recognition or an eye tracking camera 366 for tracking eyeball movement. In some embodiments, the computer device 140 includes one or more optical cameras (e.g., an RGB camera), scanners, or photo sensor units for capturing images. The computer system 300 also includes one or more output devices 312 that enable presentation of user interfaces 210 and display content includes one or more speakers and/or one or more visual displays.

The computer system 300 includes one or more sensors 360, which further includes one or more of: a plurality of electrodes 362, one or more depth sensing sensors 364, one or more eye tracking cameras 366, a biometric sensor array 368, one or more infrared sensors 370, one or more ultrasonic sensors 372, one or more ambient sensors 374, one or more motion sensors (e.g., six degree of freedom (6DOF) position and motion sensors 376, one or more outward camera 378, and one or more directional microphones 380. It is noted that the one or more sensors 360 are also included in the input device 310 and used to collect data to the computer system 300.

Memory 306 includes high-speed random-access memory, such as DRAM, SRAM, DDR RAM, or other random-access solid state memory devices; and, optionally, includes non-volatile memory, such as one or more magnetic disk storage devices, one or more optical disk storage devices, one or more flash memory devices, or one or more other non-volatile solid state storage devices. Memory 306, optionally, includes one or more storage devices remotely located from one or more processing units 302. Memory 306, or alternatively the non-volatile memory within memory 306, includes a non-transitory computer readable storage medium. In some embodiments, memory 306, or the non-transitory computer readable storage medium of memory 306, stores the following programs, modules, and data structures, or a subset or superset thereof:
Operating system 314 including procedures for handling various basic system services and for performing hardware dependent tasks;
Network communication module 316 for connecting each server 102 or computer device 140 to other devices (e.g., server 102, computer device 140, or storage 106) via one or more network interfaces 304 (wired or wireless) and one or more communication networks 108, such as the Internet, other wide area networks, local area networks, metropolitan area networks, and so on;
User interface module 318 for enabling presentation of information (e.g., a graphical user interface for application(s) 324, widgets, websites and web pages thereof, and/or games, audio and/or video content, text, etc.) at each computer device 140 via one or more output devices 312 (e.g., displays, speakers, etc.);
Input processing module 320 for detecting one or more user inputs or interactions from one of the one or more input devices 310 and interpreting the detected input or interaction;
Web browser module 322 for navigating, requesting (e.g., via HTTP), and displaying websites and web pages thereof includes a web interface for logging into a user account associated with a computer device 140 or another electronic device, controlling the computer device if associated with the user account, and editing and reviewing settings and data that are associated with the user account;
One or more user applications 324 for execution by the computer system 300 (e.g., games, social network applications, smart home applications, extended reality application, and/or other web or non-web-based applications for controlling another electronic device and reviewing data captured by such devices), where in some embodiments, an eyewear fitting application 326 can be executed to implement eyewear fitting, and has a plurality of user accounts associated with a plurality of users 120 (e.g., technician users and eyewear users), and in some embodiments, a visual assessment application 328 can be executed to evaluate eyesight of a patient user, and has a plurality of user accounts associated with a plurality of users 120 (e.g., an optometrist user, a patient user);
Data processing module 330 for processing data associated with the user applications 324, e.g., using machine learning models 350;
Model training Module 332 for obtaining training data 346 and training machine learning models 350; and
One or more databases 340 for storing at least data including one or more of:
   Device settings 334 including common device settings (e.g., service tier, device model, storage capacity, processing capabilities, communication capabilities, etc.) of the computer system 300;
   User account information 336 for the one or more user applications 324, e.g., user names, security questions, account history data, user preferences, and predefined account settings, where in some embodiments, the user account information 336 includes facial measurements and one or more virtual fitting parameters associated with associated with a user account of an eye fitting application 326, and in some embodiments, the user account information 336 includes visual stimuli 338, sensor data 342, and vision test results 344 associated with a user account of a visual assessment application 328; and
   Machine learning models 350 including parameters (e.g., weights, biases) used to implement vision test or select eyewear for eyewear users.

Each of the above identified elements may be stored in one or more of the previously mentioned memory devices and correspond to a set of instructions for performing a function described above. The above identified modules or programs (i.e., sets of instructions) need not be implemented as separate software programs, procedures, modules or data structures, and thus various subsets of these modules may be combined or otherwise rearranged in various embodiments. In some embodiments, memory 306, optionally, stores a subset of the modules and data structures identified above. Furthermore, memory 306, optionally, stores additional modules and data structures not described above.

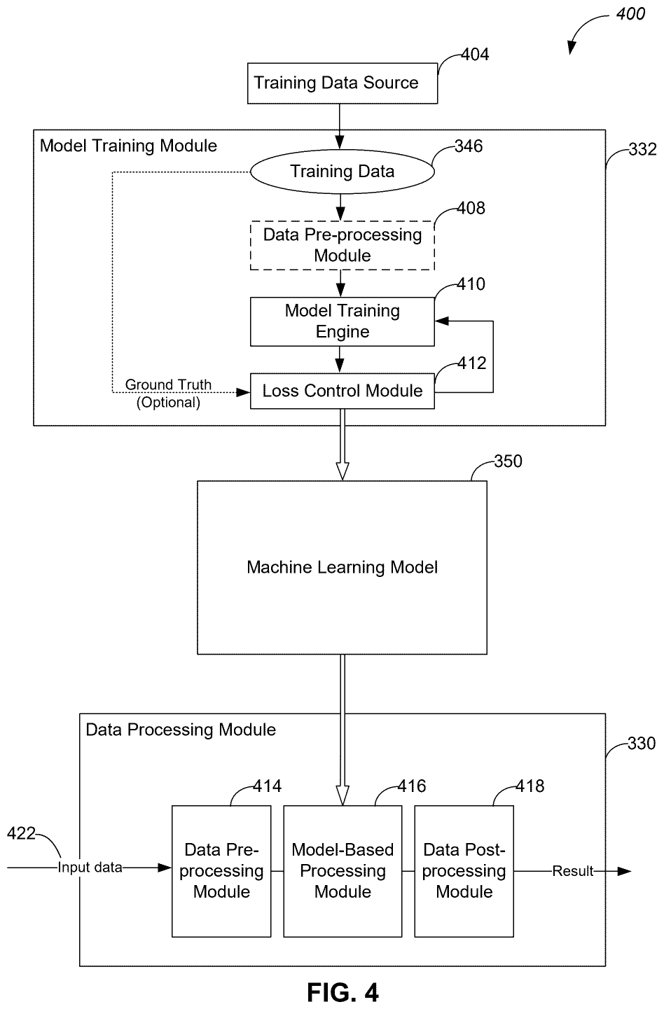

FIG. 4 is a block diagram of a machine learning system 400 for training and applying machine learning models 350 (e.g., for glass making), in accordance with some embodiments. The machine learning system 400 includes a model training module 332 establishing one or more machine learning models 350 and a data processing module 330 for processing input data 422 using the machine learning model 350. In some embodiments, both the model training module 332 and the data processing module 330 are located within a computer device 140 (e.g., a VR headset), while a training data source 404 provides training data 346 to the computer device 140. In some embodiments, the training data source 404 is the data obtained from the computer device 140 itself, from a server 102, from storage 106, or from another electronic device or computer device 140. Alternatively, in some embodiments, the model training module 332 is located at a server 102, and the data processing module 330 is located in a computer device 140. The server 102 trains the machine learning model 350 and provides the trained models 350 to the computer device 140 to process real-time input data 422 detected by the computer device 140. In some embodiments, the training data 346 provided by the training data source 404 include a standard dataset widely used to train machine learning models 350. The input data 422 further includes sensor data. Further, in some embodiments, a subset of the training data 346 is modified to augment the training data 346. The subset of modified training data is used in place of or jointly with the subset of training data 346 to train the machine learning models 350.

In some embodiments, the model training module 332 includes a model training engine 410, and a loss control module 412. Each machine learning model 350 is trained by the model training engine 410 to process corresponding input data 422 to implement a respective task. Specifically, the model training engine 410 receives the training data 346 corresponding to a machine learning model 350 to be trained and processes the training data to build the machine learning model 350. In some embodiments, during this process, the loss control module 412 monitors a loss function comparing the output associated with the respective training data item to a ground truth of the respective training data item. In these embodiments, the model training engine 410 modifies the machine learning models 350 to reduce the loss, until the loss function satisfies a loss criteria (e.g., a comparison result of the loss function is minimized or reduced below a loss threshold). The machine learning models 350 are thereby trained and provided to the data processing module 330 of a computer device 140 to process real-time input data 422 from the computer device 140.

In some embodiments, the model training module 402 further includes a data pre-processing module 408 configured to pre-process the training data 346 before the training data 346 is used by the model training engine 410 to train a machine learning model 350. For example, an image pre-processing module 408 is configured to format patients'eye images in the training data 346 into a predefined image format. For example, the preprocessing module 408 may normalize the images to a fixed size, resolution, or contrast level. In another example, an image pre-processing module 408 extracts a region of interest (ROI) corresponding to an eye area.

In some embodiments, the model training module 332 uses supervised learning in which the training data 346 is labelled and includes a desired output for each training data item (also called the ground truth in some situations). In some embodiments, the desirable output is labelled manually by people or labelled automatically by the model training model 332 before training. In some embodiments, the model training module 332 uses unsupervised learning in which the training data 346 is not labelled. The model training module 332 is configured to identify previously undetected patterns in the training data 346 without pre-existing labels and with little or no human supervision. Additionally, in some embodiments, the model training module 332 uses partially supervised learning in which the training data is partially labelled.

In some embodiments, the data processing module 330 includes a data pre-processing module 414, a model-based processing module 416, and a data post-processing module 418. The data pre-processing modules 414 pre-processes input data 422 based on the type of the input data 422. In some embodiments, functions of the data pre-processing modules 414 are consistent with those of the pre-processing module 408 and convert the input data 422 into a predefined data format that is suitable for the inputs of the model-based processing module 416. The model-based processing module 416 applies the trained machine learning model 350 provided by the model training module 332 to process the pre-processed input data 422. In some embodiments, the model-based processing module 416 also monitors an error indicator to determine whether the input data 422 has been properly processed in the machine learning model 350. In some embodiments, the processed input data is further processed by the data post-processing module 418 to create a preferred format or to provide additional information that can be derived from the processed input data. The data processing module 330 uses the processed input data to make eyewear glasses for a patient user.

Examples of the machine learning model 350 include, but are not limited to, an eye trajectory model, an eye position model, an ocular microtremor model, a response analysis model, a response analysis model, a biomedical data model, and medical information models.

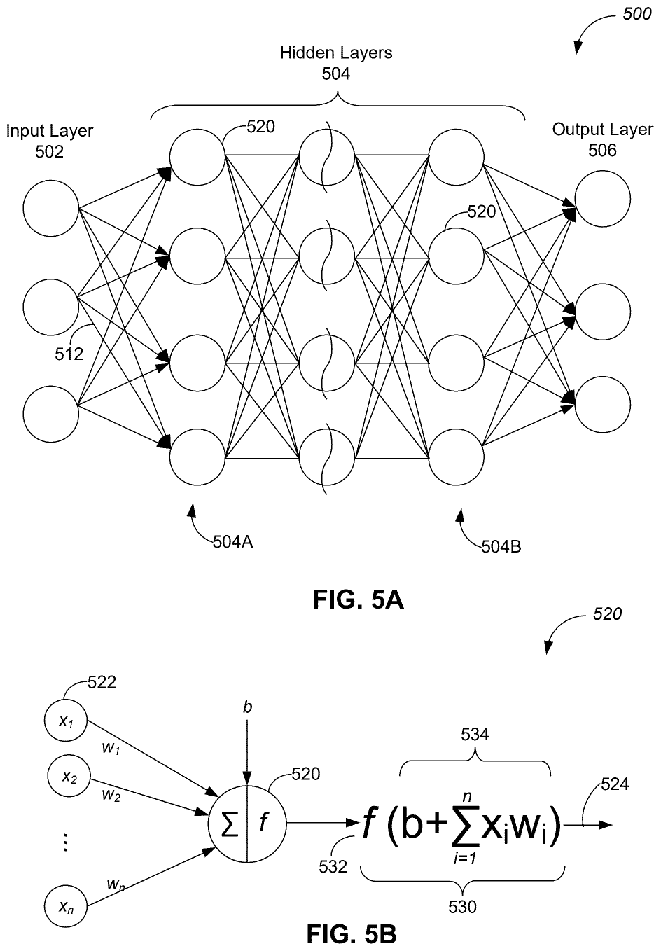

FIG. 5A is a structural diagram of an example neural network 500 applied to process input data in a machine learning model 350, in accordance with some embodiments, and FIG. 5B is an example node 520 in the neural network 500, in accordance with some embodiments. It should be noted that this description is used as an example only, and other types or configurations may be used to implement the embodiments described herein. The machine learning model 350 is established based on the neural network 500. A corresponding model-based processing module 416 applies the machine learning model 350 including the neural network 500 to process input data 422 that has been converted to a predefined data format. The neural network 500 includes a collection of nodes 520 that are connected by links 512. Each node 520 receives one or more node inputs 522 and applies a propagation function 530 to generate a node output 524 from the one or more node inputs. As the node output 524 is provided via one or more links 512 to one or more other nodes 520, a weight w associated with each link 512 is applied to the node output 524. Likewise, the one or more node inputs 522 are combined based on corresponding weights w₁, w₂, w₃, and w₄ according to the propagation function 530. In an example, the propagation function 530 is computed by applying a non-linear activation function 532 to a linear weighted combination 534 of the one or more node inputs 522.

The collection of nodes 520 is organized into layers in the neural network 500. In general, the layers include an input layer 502 for receiving inputs, an output layer 506 for providing outputs, and one or more hidden layers 504 (e.g., layers 504A and 504B) between the input layer 502 and the output layer 506. A deep neural network has more than one hidden layer 504 between the input layer 502 and the output layer 506. In the neural network 500, each layer is only connected with its immediately preceding and/or immediately following layer. In some embodiments, a layer is a “fully connected” layer because each node in the layer is connected to every node in its immediately following layer. In some embodiments, a hidden layer 504 includes two or more nodes that are connected to the same node in its immediately following layer for down sampling or pooling the two or more nodes. In particular, max pooling uses a maximum value of the two or more nodes in the layer for generating the node of the immediately following layer.

In some embodiments, a convolutional neural network (CNN) is applied in a machine learning model 350 to process input data. The CNN employs convolution operations and belongs to a class of deep neural networks. The hidden layers 504 of the CNN include convolutional layers. Each node in a convolutional layer receives inputs from a receptive area associated with a previous layer (e.g., nine nodes). Each convolution layer uses a kernel to combine pixels in a respective area to generate outputs. For example, the kernel may be to a 3×3 matrix including weights applied to combine the pixels in the respective area surrounding each pixel. Video or image data is pre-processed to a predefined video/image format corresponding to the inputs of the CNN. In some embodiments, the pre-processed video or image data is abstracted by the CNN layers to form a respective feature map. In this way, video and image data can be processed by the CNN for video and image recognition or object detection.

In some embodiments, a recurrent neural network (RNN) is applied in the machine learning model 350 to process input data 422. Nodes in successive layers of the RNN follow a temporal sequence, such that the RNN exhibits a temporal dynamic behavior. In an example, each node 520 of the RNN has a time-varying real-valued activation. It is noted that in some embodiments, two or more types of input data are processed by the data processing module 330, and two or more types of neural networks (e.g., both a CNN and an RNN) are applied in the same machine learning model 350 to process the input data jointly.

The training process is a process for calibrating all of the weights wi for each layer of the neural network 500 using training data 346 that is provided in the input layer 502. The training process typically includes two steps, forward propagation and backward propagation, which are repeated multiple times until a predefined convergence condition is satisfied. In the forward propagation, the set of weights for different layers are applied to the input data and intermediate results from the previous layers. In the backward propagation, a margin of error of the output (e.g., a loss function) is measured (e.g., by a loss control module 412), and the weights are adjusted accordingly to decrease the error. The activation function 532 can be linear, rectified linear, sigmoidal, hyperbolic tangent, or other types. In some embodiments, a network bias term b is added to the sum of the weighted outputs 534 from the previous layer before the activation function 532 is applied. The network bias b provides a perturbation that helps the neural network 500 avoid over fitting the training data. In some embodiments, the result of the training includes a network bias parameter b for each layer.

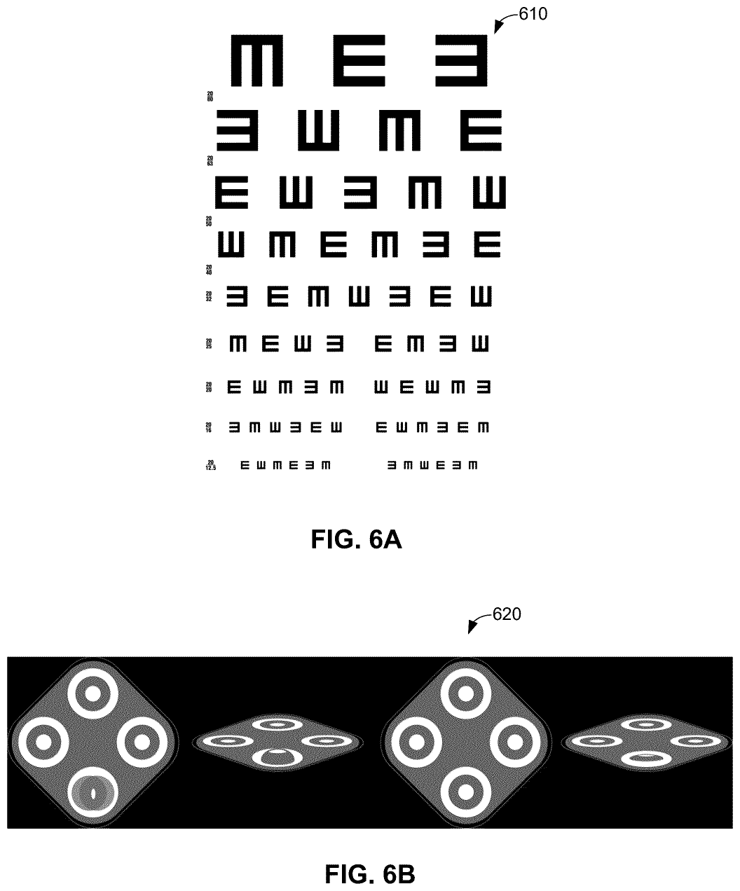

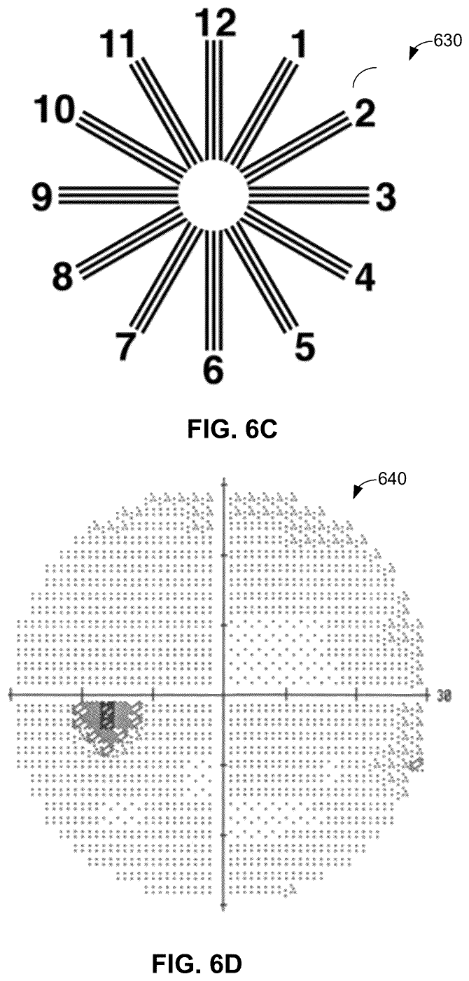

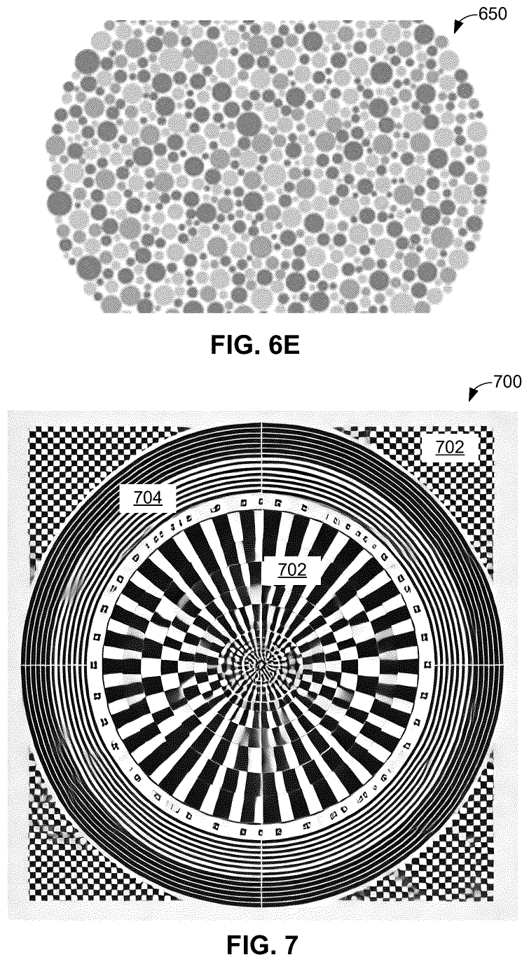

In some embodiments of the present disclosure, a vision test is implemented in a headset device 140D configured to display a user interface creating a three-dimensional (3D) virtual environment. Examples of a vision test implemented in the 3D virtual environment include, but are not limited to a visual acuity test, a visual field test, a visual depth test, a color blindness test, a retinoscopy, a test for stereopsis, a refraction test, an astigmatism test, and a contact lens exam. FIG. 6A is an example “tumbling E” chart 610 applied in a visual acuity test, in accordance with some embodiments. FIGS. 6B, 6C, 6D, and 6E are example patterns 620, 630, 640, and 650 applied in an astigmatism test, a stereopsis test, a visual field test, and a color blindness test, in accordance with some embodiments.

FIG. 7 is another example visual pattern 700 applied to test visual acuity and astigmatism, in accordance with some embodiments. The visual pattern 700 integrates a grid pattern 702 and concentric rings 704. The grid pattern 702 may include evenly spaced horizontal and vertical lines, creating a checkerboard pattern. The grid pattern 702 may be configured to identify distortions in straight lines, which can indicate issues with visual acuity and astigmatism. The concentric rings 704 may expand outward from a center of the visual pattern 700 and can assist in detecting radial distortions, which are common indicators of astigmatism. The visual pattern 700 may be depicted in high-contrast black and white, which ensures maximum clarity and reduces the potential for color-related distortions, making it easier to detect any visual impairment or defect.

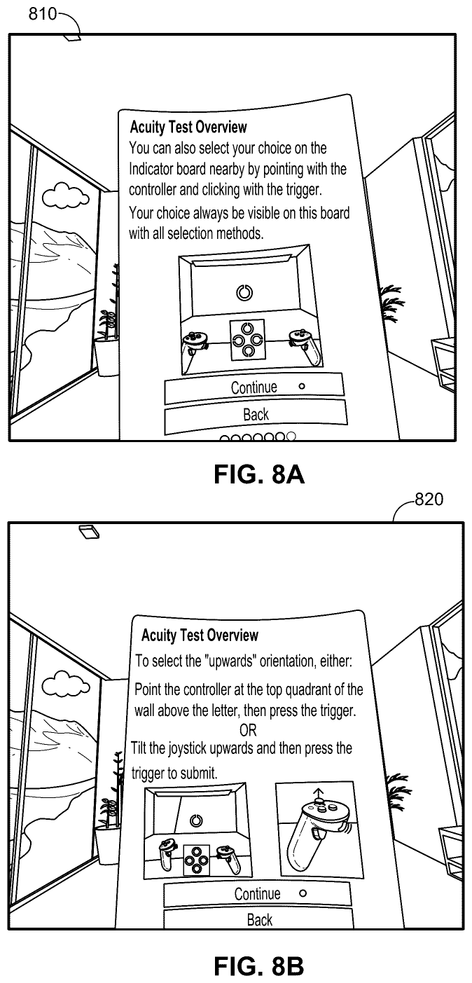

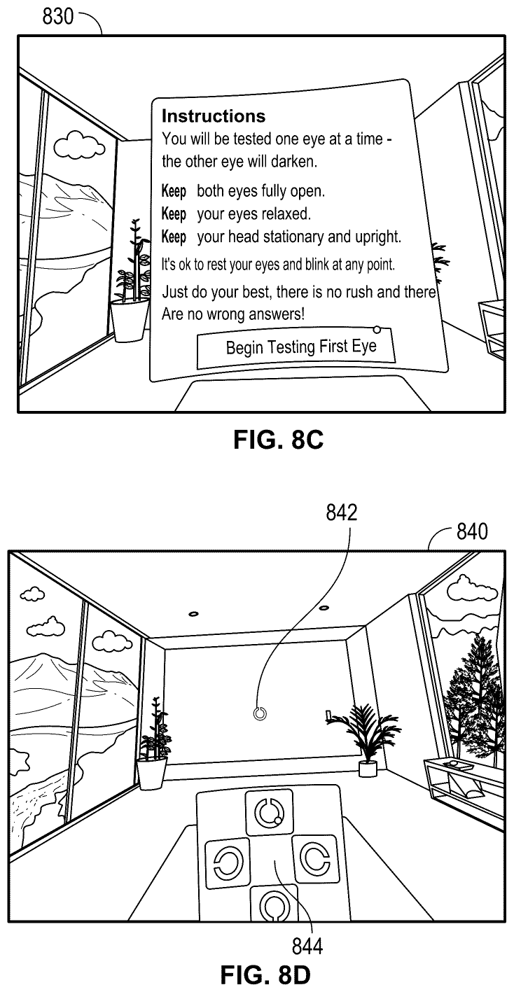

FIGS. 8A-8D include four diagrams of example graphical user interfaces 810, 820, 830, and 840 rendered to determine a visual acuity score in a virtual environment created by a headset device 140D, in accordance with some embodiments. The user interface 810 displays an information page including instructions on controlling a headset device 140D to select one of a plurality of optotype candidates to match a target optotype displayed in the virtual environment. The user interface 820 displays an information page including two optional ways of using the controller to select the one of the plurality of optotype candidates. The user interface 830 displays an information page including general guidelines on a visual acuity assessment process. The user interface 840 displays an optotype 842 that is projected on a screen that has a first distance L1 from a user's position in the virtual environment. In a second distance L2 near the user, a selection panel 844 including a plurality of optotype candidates is displayed, prompting the user to select one of the optotype candidates that matches the optotype 842. In some embodiments, in response to a user selection of the one of the optotype candidates, the optotype 842 displayed in the first distance L1 is updated with a new optotype 842. Further, in some embodiments, the new optotype 842 spins at a fast rate for a shortened duration of time (e.g., 2 seconds), before it settles in place of the original optotype 842. In an example, the optotype 842 spins and gradually shrinks in size during the shortened duration of time.

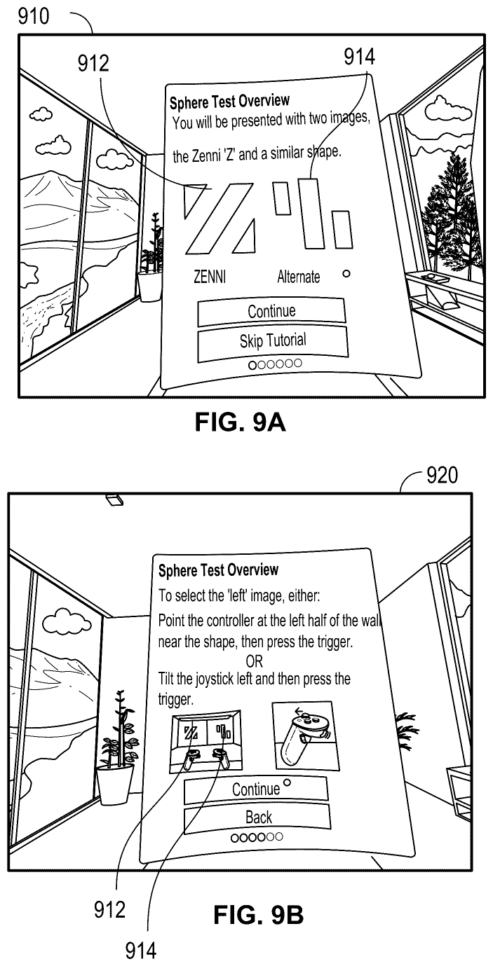

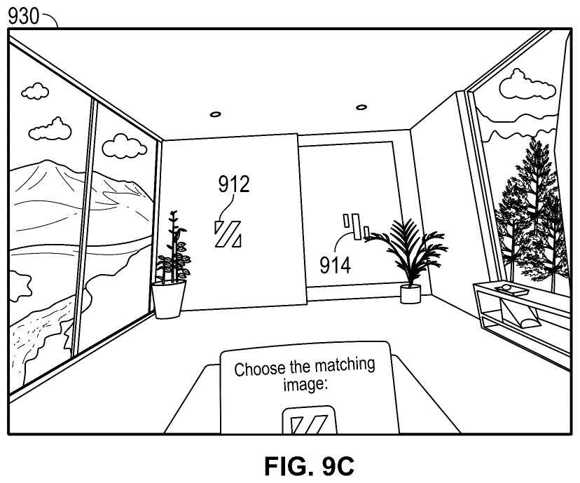

FIGS. 9A-9C include three diagrams of example graphical user interfaces 910, 920, and 930 rendered to determine a nearsighted or farsighted power in a virtual environment created by a headset device 140D, in accordance with some embodiments. The user interface 910 displays an information page explaining that two target optotypes 912 and 914 are displayed in the virtual environment. The user interface 920 displays an information page including two optional ways of using the controller to select one of the two target optotypes 912 and 914. The user interface 930 displays two target optotypes 912 and 914 that are projected on a screen that has a first distance L1 from a user's position in the virtual environment. In this example, the target optotype 912 located on the left is highlighted (e.g., by being displayed in a colored background). In a second distance L2 near the user, a confirmation panel 932 is displayed, prompting the user to select one of the two target optotypes 912 and 914. In some embodiments, in response to a user selection of the one of the two target optotypes 912 and 914, the two target optotypes 912 and 914 displayed in the first distance L1 is updated with a new pair of two target optotypes 912 and 914. Further, in some embodiments, each optotype 912 or 914 spins at a fast rate for a shortened duration of time (e.g., 2 seconds), before it settles in place of the original optotype 912 or 914. In an example, the optotype 912 or 914 spins and gradually shrinks in size during the shortened duration of time.

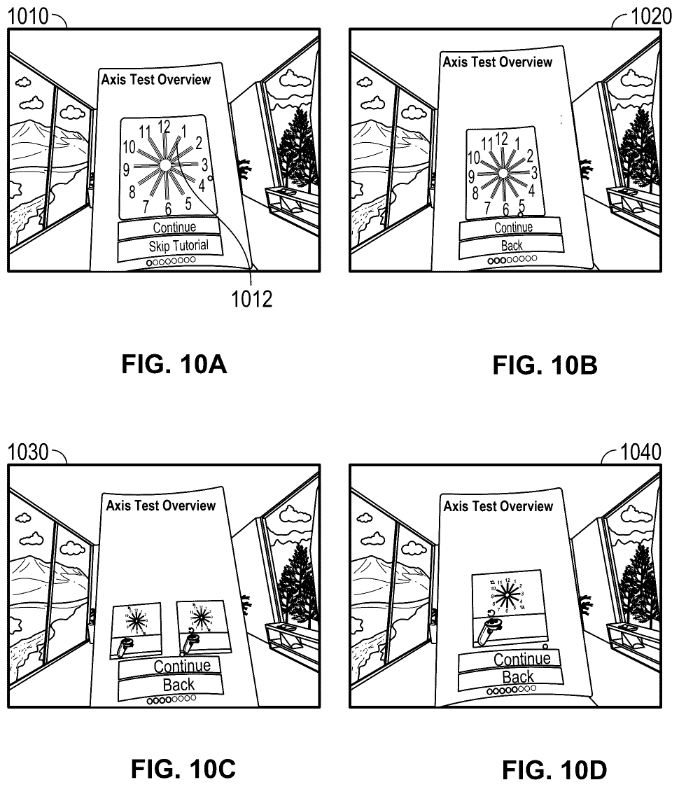

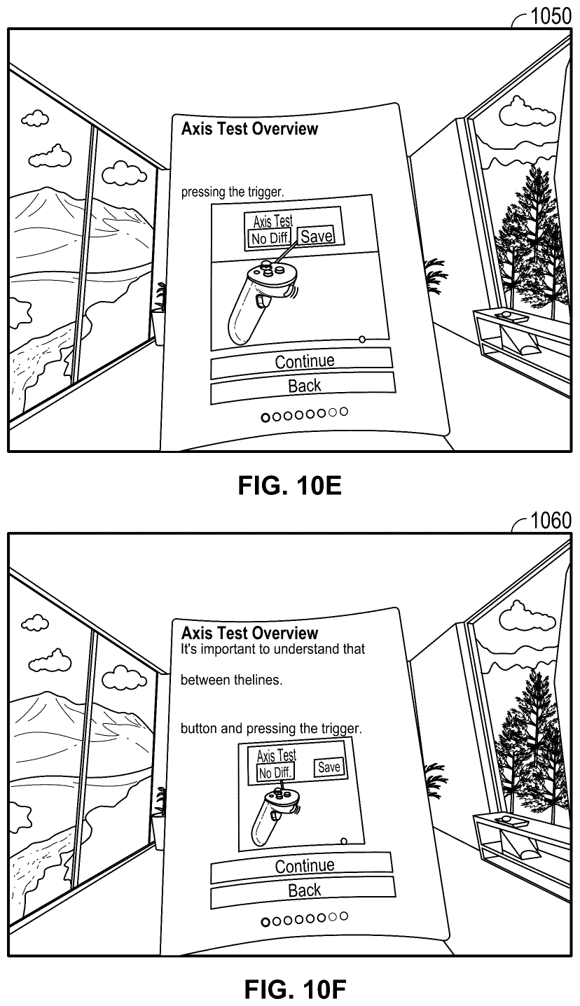

FIGS. 10A-10F include six diagrams of example graphical user interfaces 1010, 1020, 1030, 1040, 1050, and 1060 rendered to determine eye stigmatism in a virtual environment created by a headset device 140D, in accordance with some embodiments. The user interface 1010 displays an information page explaining that a clock diagram of converging numbered lines 1012 (which is a type of optotype) is displayed in the virtual environment. The user interface 1020 displays an information page explaining what is selected on the clock diagram of converging numbered lines 1012 displayed in the virtual environment. The user interface 1030 displays an information page including two optional ways of using the controller to select lines on the clock diagram of converging numbered lines 1012. The user interface 1040 displays an information page explaining a situation having equally clear lines on the clock diagram of converging numbered lines 1012. The user interface 1050 displays an information page including an instruction using the controller to submit a selection. The user interface 1060 displays an information page including an instruction using the controller to indicate that no difference is observed on the clock diagram of converging numbered lines 1012.

Some embodiments of a VR system are configured to enhance administration and experience of vision tests. The VR system includes a headset device 140D equipped with a display (sometimes referred to as a head-mounted display (HMD)). In some embodiments, the headset device 140D includes and one or more sensors for tracking one or more of eye movement, head orientation, and/or hand gestures of a user wearing the headset device 140D. In some embodiments, the headset device 140D is configured to execute a vision assessment application 328 configured to adaptively manage a sequence of vision tests based on the user's condition. In some embodiments, the headset device 140D is communicatively coupled to a server 102 configured to execute a server-side module for the vision assessment application 328, thereby managing the sequence of vision tests jointly with a device-side module of the vision assessment application 328 executed on the headset device. The vision assessment application 328 is configured to generate a virtual reality (VR) user interface corresponding to a three-dimensional (3D) virtual environment and render visual stimuli 338 in this 3D virtual environment. A range of different vision tests are conducted based on the visual stimuli within an immersive VR space.

In some embodiments, a headset device 140D includes one or more processors 302 and memory 306 storing instructions to execute the vision assessment application 328 for rendering visual stimuli 338 in an output device 312 (e.g., a display) and processing sensor data 342 collected from the sensors 360 in response to the visual stimuli 338. The sensor data 342 may be processed to determine vision test results 344 (e.g., eye movement patterns, response times, and visual perception accuracy) for the user. Further, in some embodiments, VR technology facilitates a personalized control scheme for navigating the vision tests. The personalized control scheme enables the user to interact with the test environment through intuitive hand gestures and eye movements, thereby providing a natural and engaging testing experience. The vision tests may be customized based on individual users'requirements and accommodate a wide range of vision impairments.

In some embodiments, the vision test results 344 are used to generate comprehensive reports on the user's visual performance. For example, the headset device 140D employs a deep learning model that correlates micro-expression data with vision test results 344 to provide holistic assessment of the user's ocular health. In some situations, the vision test results 344 are applied to identify vision conditions of the user and track changes of the vision conditions over time, thereby offering valuable insights to healthcare providers. In various embodiments of this application, eye images are captured and used to determine eye movement information automatically and without user intervention, which is an efficient solution to provide reliable supplemental information that cannot be provided by the user's active responses to visual stimuli.

### Example Vision Test System

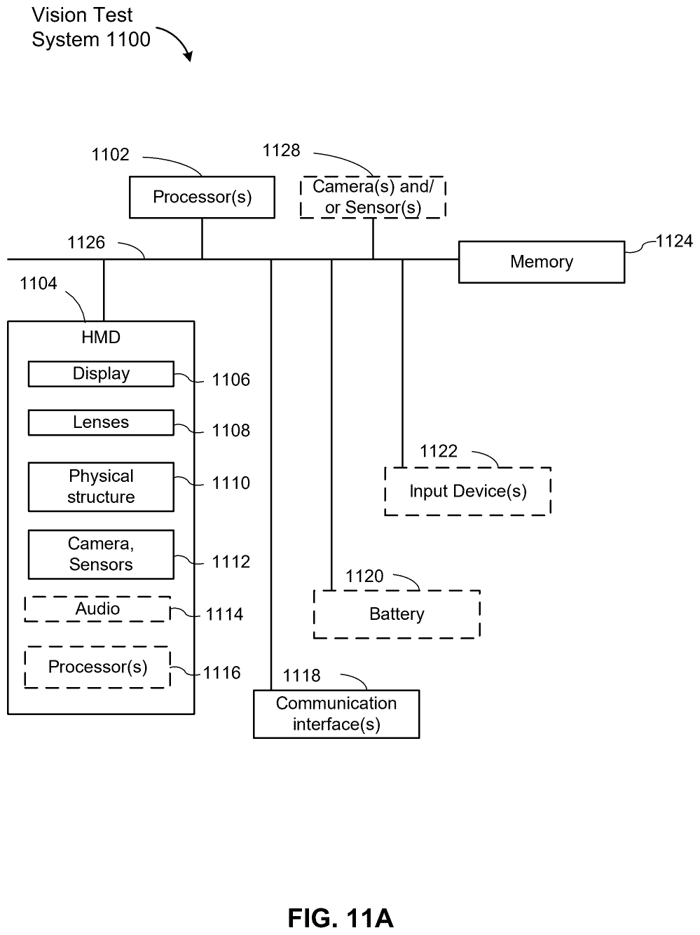

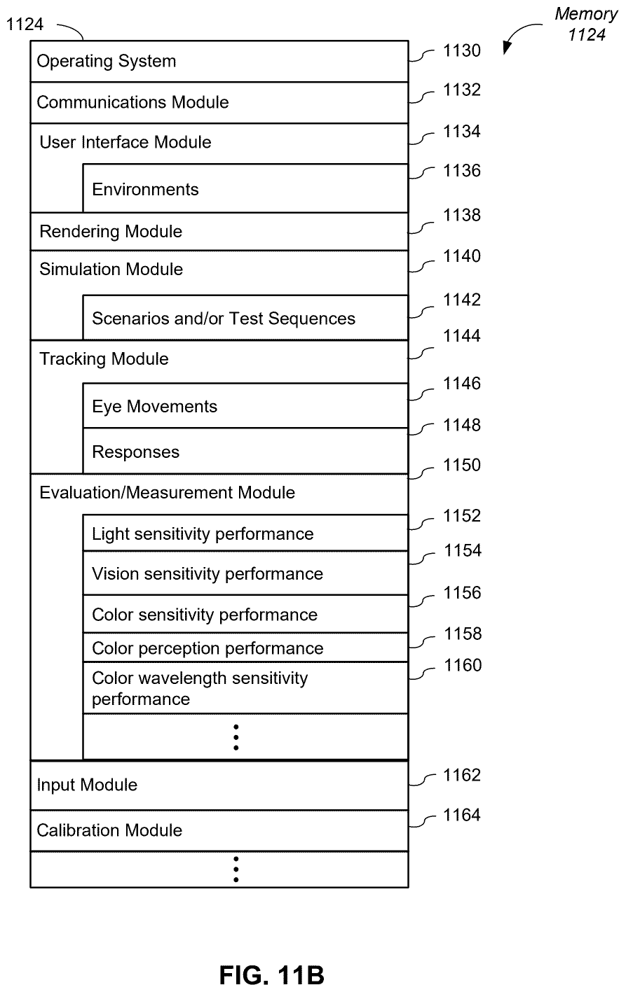

FIG. 11A is a diagram showing an example vision test system 1100, in accordance with some embodiments. The vision test system 1100 is implemented using a computer device (e.g., headset device 140D). The computer device includes one or more processors 1102, memory 1124 storing instructions to be implemented by the processor(s) 1102, a head-mounted display 1104, one or more network or other communications interfaces 1118, and one or more communication buses 1126 for interconnecting these and other optional components. The communication buses 1126 may include circuitry that interconnects and controls communications between system components.

The HMD 1104 may include a display 1106 (e.g., one or more high-resolution screens, one or more lenses 1108 (to focus and/or shape display images), cameras and/or sensors 1112 (e.g., outward camera 378, eye-tracking camera 366), and/or a physical structure 1110 (e.g., a structure that holds the components and configured to be worn on a head). The HMD 1104 optionally includes audio devices 1114 and one or more processors 1116 (instead of or in addition to the processors 1102, to implement instructions in the memory 1124). One or more cameras and/or sensors 1128 may be optionally included in some embodiments, instead of or in addition to the cameras and/or sensors 1112 integrated within the HMD 1104. The HMD may include, for example, high-resolution displays (e.g., 4K per eye), wide field of view (e.g., minimum 110 degrees), and/or adjustable interpupillary distance. The eye-tracking sensors can include, for example, high-precision infrared cameras, have a tracking frequency of 120 Hz or higher, have a latency of less than 5 milliseconds, and/or have an accuracy of sub-millimeter precision and/or 0.1 degrees in gaze direction.

In some embodiments, the computer device also includes one or more input devices 1122 (e.g., controllers and/or hand-tracking sensors). In some embodiments, the computer device also includes a battery 1120 (e.g., for standalone headsets). In some embodiments, the input device/mechanism 1122 includes a keyboard. In some embodiments, the input device/mechanism 1122 includes a “soft” keyboard, which is displayed as needed on the display 1106, for example, to enable a user to “press keys” that appear on the display 1106. In various embodiments, the communication interface(s) 1118 includes Wi-Fi, Bluetooth, and/or wired connections. In some embodiments, the input devices 1122 may include VR controllers and/or hand-tracking sensors.

In some embodiments, the memory 1124 includes high-speed random-access memory, such as DRAM, SRAM, DDR RAM, and/or other random-access solid state memory devices. In some embodiments, the memory 1124 includes non-volatile memory, such as one or more magnetic disk storage devices, optical disk storage devices, flash memory devices, or other non-volatile solid state storage devices. In some embodiments, the memory 1124 includes one or more storage devices remotely located from the processor(s) 1102. The memory 1124, or alternatively the non-volatile memory device(s) within the memory 1124, comprises a computer readable storage medium. Memory for headsets include, for example, Random-Access Memory (RAM), such as Low Power Double Data Rate RAM (LPDDR), used for running the operating system, applications, and/or handling real-time data processing. Memory 1124 may also include storage memory, such as flash memory, similar to smartphones (e.g., eMMC or UFS), for storing the operating system, applications, and/or user data. Video memory, often integrated with the GPU in mobile chipsets, can be used to handle graphics processing tasks. Cache memory, such as Static RAM (SRAM), can be used for high-speed memory used by the processors 1102 for quick data access.

Referring to FIG. 11B, in some embodiments, the memory 1124, or the computer readable storage medium of the memory 1124, stores the following programs, modules, and data structures, or a subset thereof.
an operating system 1130, which includes procedures for handling various basic system services and for performing hardware dependent tasks;
a communications module 1132, which is used for connecting the computing device to other computers and devices via the one or more communication network interfaces 1118 (wired or wireless) and/or via one or more communication networks, such as the Internet, other wide area networks, local area networks, metropolitan area networks, and so on;
a user interface module 1134 (sometimes referred to as the UI module 1134) for managing user interaction with VR/AR environments 1136 (sometimes referred to as three-dimensional virtual environments, photorealistic environments) and/or having system controls. This can include home environment, allowing users to launch apps, adjust settings, and/or navigate menus using virtual pointers or hand gestures;
a rendering module 1138 for handling the creation and/or display of 3D graphics in real-time. This can include a rendering pipeline, for example Unity's VR rendering pipeline, for optimizing frame rates and/or reducing latency for smooth VR/AR experiences;
a simulation module 1140 for creating and/or managing the rules, physics, and/or behaviors within the virtual environment. This can, for example, include PhysX in VR games, simulating realistic object interactions and gravity effects. The simulation module 1140 may include one or more scenarios and/or test sequences 1142;
a tracking module 1144 for processing sensor data to determine the position and orientation of the headset and/or controllers. The tracking module can track eye movements 1146 and/or responses 1148 (sometimes referred to as user responses), which may include, for example response times. In various embodiments, the eye movements 1146 includes, for example, gaze direction, fixation points, blink rate, squinting, and/or pupillary responses;
an evaluation and/or measurement module 1150 for analyzing tracked data, user interactions and/or system performance for optimization and/or adaptation and feedback to determine and/or measure, for example, light sensitivity performance 1152, vision sensitivity performance 1154, color sensitivity performance 1156, color perception performance 1158, and/or color wavelength sensitivity performance 1160. In some embodiments, the evaluation module performs real-time data processing and/or analysis, calculates performance metrics (e.g., reaction times, error rates), and/or assesses color perception and/or wavelength sensitivity. In some embodiments, the module 1150 can include one or more recommendation engines for AI-driven analysis for personalized recommendations, and/or adaptive eyewear and lens tint suggestions. In some embodiments, the module 1150 also includes a reporting system for report generation and/or visual field mapping and/or color sensitivity profiling;
an input module 1162 for interpreting and/or processing user input from various sources (e.g., controllers, hand tracking, voice commands). This module can include hand tracking software, translating hand and finger movements into VR interactions; and/or
a calibration module 1164 for alignment of virtual and physical elements, often including initial setup procedures, for calibrating the device and/or experimental setups based on user data, which can include setup, and/or guiding users through the process of defining their viewing and/or test area and/or calibrating controllers.

The UI module 1134 may generate interactive visual elements that allow users to navigate and interact with the highly realistic 3D virtual world. This may include creating menus and buttons that appear to exist within a 3D space, implementing gesture-based controls that feel natural in the virtual world, designing visual feedback that matches the aesthetic of the environment, and/or integrating information displays seamlessly with the surroundings. The UI module 1134 may utilize various implementation methods, such as game engines (e.g., Unity, Unreal Engine) for UI implementation and integration, and/or 3D modeling software for creating UI assets.

The processing may include processing on host computers for tethered VR headsets, may include on-device processing for standalone VR/AR headsets, and/or cloud processing for computationally intensive tasks. In various embodiments, the UI module 1134 enhances user immersion and presence by, for example, creating UI elements that look and feel like they belong in the photorealistic environment, implementing holographic displays or interactive physical objects, and/or supporting interaction through VR controllers or hand tracking. In some embodiments, the UI module 1134 adapts the UI to different types of virtual environments, ensuring consistency and usability across various scenarios. In some embodiments, the UI module 1134 also handles user input (e.g., in collaboration with an input module, described below) through multiple modalities, including hand tracking, eye tracking, and controller input, to facilitate seamless interaction with the generated UI.

In some embodiments, the rendering module 1138 integrates the VR user interface elements with the photorealistic environment, ensuring proper depth, occlusion, and lighting interactions. In some embodiments, the rendering module 1138 implements stereo rendering techniques to create a sense of depth and dimensionality for the UI elements when displayed on the HMD. In some embodiments, the rendering module 1138 applies distortion correction and lens-specific optimizations to ensure the UI is properly displayed on the HMD's optics. In some embodiments, the rendering module 1138 utilizes techniques like foveated rendering to optimize UI rendering performance, particularly for resource-intensive photorealistic environments. In some embodiments, the rendering module 1138 handles dynamic UI updates and animations in real-time, maintaining consistent frame rates crucial for comfortable VR experiences. In some embodiments, the rendering module 1138 implements anti-aliasing and other image quality enhancements specific to HMD displays to ensure crisp, readable UI elements.

In various embodiments, the one or more scenarios 1142 can include real-world scenarios, dynamic real-world visual experiences, test sequences with progressively finer details, real-world motion and target recognition visual tasks, and/or various visual scenarios (including, for example, scenarios with different lighting conditions). In some embodiments, the simulation module 1140 may be further configured to generate and manage real-world scenarios in the VR user interface, such as simulating everyday activities or specific professional environments. In some embodiments, the simulation module 1140 may be further configured to create and control testing sequences that progressively introduce finer details and objects at varying depths within the three-dimensional virtual environment, allowing for comprehensive visual acuity assessment.

In some embodiments, the simulation module 1140 may be further configured to simulate dynamic real-world visual experiences by incorporating moving objects, changing environments, and interactive elements that respond to user actions. In some embodiments, the simulation module 1140 may be further configured to implement real-world motion and target recognition tasks, such as tracking moving objects or identifying specific targets within complex visual scenes. In some embodiments, the simulation module 1140 may be further configured to generate visual scenarios that require focus adjustments, simulating the need to shift focus between near and far objects in the virtual environment.

In some embodiments, the simulation module 1140 may be further configured to create a diverse range of visual scenarios, each designed to test different aspects of vision or simulate specific real-world conditions. In some embodiments, the simulation module 1140 may be further configured to implement lighting simulation algorithms to create visual scenarios with varying lighting conditions, including daylight, twilight, indoor lighting, and challenging low-light situations. In some embodiments, the simulation module 1140 may be further configured to utilize the PhysX engine or similar physics simulation tools to ensure realistic object behavior and interactions within these scenarios, enhancing the authenticity of the simulated experiences.

In some embodiments, the simulation module 1140 may be further configured to integrate with the rendering module 1138 to ensure that simulated scenarios are accurately displayed on the HMD, maintaining the intended visual fidelity and realism. In some embodiments, the simulation module 1140 may be further configured to allow customization and parametric control of scenarios, enabling the creation of tailored visual experiences for specific testing or training purposes.

For eye testing purposes, some embodiments track eye movements and response times with high frequency and precision. In some embodiments, for eye movements, and specifically for saccades, rapid movements of the eye between fixation points are tracked at rates of at least 100-500 Hz. This high frequency helps capture the quick and brief nature of these movements accurately. For fixations, periods where the eyes are relatively stationary and focused on a single point are tracked at slightly lower rates, but typically in the range of 50-100 Hz, to ensure precise measurement of duration and stability. For smooth pursuit (e.g., movements where the eyes smoothly follow a moving object), eye movements are also tracked at high rates (100-200 Hz) to accurately capture the speed and trajectory of the eye movements.

In some embodiments, the high-precision eye tracking is achieved through a combination of hardware and software algorithms. For example, the hardware may include multiple infrared cameras strategically positioned around each eye, capturing images at a minimum of 1,000 frames per second. These cameras may use custom-designed sensors with a minimum resolution (e.g., at least 5 megapixels) for detailed capture of eye movements. The software may use computer vision algorithms, including, for example, convolutional neural networks (CNNs), for pupil detection and/or corneal reflection tracking. These algorithms may process the high-frame-rate imagery in real-time, employing, for example, parallel computing techniques to maintain low latency. Some embodiments use a predictive model to anticipate eye movements, further reducing effective latency. Calibration routines, for example, may employ active learning methods to rapidly adapt to individual eye physiologies. Using such a combination of high-speed imagery, advanced image processing, and/or predictive modeling some embodiments can track eye movements with sub-millimeter precision, a latency of less than 5 milliseconds, and/or an operational frequency exceeding 120 Hz.

In some embodiments, for response times, specifically for reaction time (e.g., the time it takes for a person to respond to a visual stimulus, such as pressing a button when a light appears), are tracked with millisecond accuracy. This typically means using sampling rates of 1000 Hz or higher to ensure precise measurement. For decision time, which may include, for example, the duration between recognizing a visual stimulus and making a decision based on, are tracked using high-frequency tracking, typically around 500-1000 Hz, to accurately capture the cognitive processing speed.

High-frequency tracking ensures that no significant movement or response detail is missed, providing a more accurate and reliable assessment of visual function. Real-world visual tasks involve rapid and complex eye movements, and high-frequency tracking allows for a more detailed analysis of how well the eyes can handle such tasks. Subtle abnormalities in eye movements or delays in response times can be early indicators of visual or neurological problems. High-frequency tracking helps in detecting these issues at an early stage. In some embodiments, for eye testing, continuous tracking of eye movements and response times is performed at high frequencies (e.g., ranging from 50 Hz to 1000 Hz) to ensure precise and comprehensive data collection. While both eye testing and VR games benefit from eye-tracking technology, the former requires much higher precision, frequency, and reliability for clinical and diagnostic purposes. In contrast, VR games prioritize user experience and real-time interaction, allowing for lower precision and frequency in tracking (e.g., 30-120 Hz).

In some embodiments, the tracking module 1144 may be further configured to continuously track eye movements and response times to visual stimuli presented in the one or more real-world scenarios simulated in the VR user interface, using the camera at high frequencies (e.g., 100-500 Hz for saccades, 50-100 Hz for fixations). In some embodiments, the tracking module 1144 may be further configured to track eye movements and response times to visual stimuli presented in the testing sequence, capturing data throughout the progression of finer details and varying depths in the three-dimensional virtual environment. In some embodiments, the tracking module 1144 may be further configured to monitor eye movements and response times to visual stimuli presented in the dynamic real-world visual experience, adapting to changing environmental conditions and moving objects within the simulation.

In some embodiments, the tracking module 1144 may be further configured to track eye movements and response times specifically for real-world motion and target recognition visual tasks, providing detailed data on how users visually engage with moving objects and identify targets in complex scenes. In some embodiments, the tracking module 1144 may be further configured to monitor dynamic focus adjustments in response to visual stimuli presented in various visual scenarios, capturing data on how quickly and accurately users can shift focus between near and far objects in the virtual environment.

In some embodiments, the tracking module 1144 may be further configured to track user interactions and responses to visual stimuli across a range of visual scenarios, including those with different lighting conditions, providing comprehensive data on visual performance under various environmental conditions. In some embodiments, the tracking module 1144 may be further configured to integrate with the simulation module 1140 to ensure synchronized tracking of eye movements and responses with the presented visual stimuli across all types of simulated scenarios.

In some embodiments, the tracking module 1144 may be further configured to process and/or analyze the collected high-frequency data in real-time, providing immediate feedback on visual performance and enabling dynamic adjustments to the testing or training protocols as needed. These enhanced tracking capabilities ensure that the system can capture detailed, precise data on eye movements and responses across a wide range of simulated scenarios, supporting comprehensive analysis of visual function and performance in virtual reality environments. In some embodiments, the tracking module 1144 may be further configured to continuously track eye movements and response times in response to visual stimuli presented in the one or more dynamic lighting scenarios. This tracking is performed using the camera at high frequencies (e.g., 100-500 Hz for saccades, 50-100 Hz for fixations) to capture rapid eye movements in changing light conditions.

In some embodiments, the tracking module 1144 may be further configured to continuously monitor and record pupil data, including pupil dilation and constriction, in response to visual stimuli presented in the one or more dynamic lighting scenarios. This pupil tracking is performed at high frequencies (e.g., 120-250 Hz) to capture subtle and rapid changes in pupil size as lighting conditions change. In some embodiments, the tracking module 1144 may be further configured to specifically track eye movements, including saccades, fixations, and smooth pursuit, in response to visual stimuli presented in the one or more dynamic lighting scenarios. This tracking captures how the eyes adapt and respond to changing light levels, moving shadows, or shifting light sources within the virtual environment.

In some embodiments, the tracking module 1144 may be further configured to synchronize the eye tracking data with the simulated lighting conditions, allowing for precise analysis of how different lighting scenarios affect eye movements, pupil reactions, and response times. In some embodiments, the tracking module 1144 may be further configured to process and analyze the collected high-frequency eye movement, pupil, and response time data in real-time, providing immediate feedback on visual performance under varying lighting conditions.

In some embodiments, the tracking module 1144 may be further configured to integrate with the simulation module 1140 to ensure that eye tracking is precisely coordinated with the dynamic changes in lighting conditions, allowing for accurate assessment of visual adaptation to light changes. These enhancements enable the system to capture detailed, time-synced data on eye movements, pupil reactions, and/or response times, specifically in relation to changing lighting conditions in the virtual environment, supporting comprehensive analysis of visual function and/or performance under various lighting scenarios.

In some embodiments, the evaluation and/or measurement module 1150 may be further configured to analyze eye movements and response times captured by the tracking module 1144 to evaluate visual acuity and perception. This may include, for example, assessing the accuracy and speed of eye movements in response to stimuli of varying sizes and contrasts. In some embodiments, the evaluation and/or measurement module 1150 may be further configured to utilize eye movement data and response times to specifically test and evaluate visual acuity, considering factors such as the minimum resolvable detail and reaction speed to visual stimuli.

In some embodiments, the evaluation and/or measurement module 1150 may be further configured to assess depth perception, motion detection, and spatial awareness by analyzing eye movements and response times during tasks that involve tracking moving objects, judging distances, and navigating 3D environments. In some embodiments, the evaluation and/or measurement module 1150 may be further configured to measure dynamic visual acuity by evaluating eye movements and response times when tracking moving targets of varying speeds and sizes, quantifying the ability to discern details of objects in motion. In some embodiments, the evaluation and/or measurement module 1150 may be further configured to analyze dynamic focus adjustment data to measure astigmatism, examining how the eyes focus on lines and shapes at different orientations and distances.

In some embodiments, the evaluation and/or measurement module 1150 may be further configured to process user interactions and responses to visual stimuli to measure and adjust for visual distortions. This may include, for example, analyzing how users perceive and interact with potentially distorted images or environments in the VR interface. In some embodiments, the evaluation and/or measurement module 1150 may be further configured to evaluate user interactions and responses in low-light scenarios to measure night blindness, assessing visual performance and adaptation in simulated nighttime or dim lighting conditions. In some embodiments, the evaluation and/or measurement module 1150 may be further configured to integrate with the simulation module 1140 to ensure that evaluations and measurements are precisely correlated with the specific visual stimuli and environmental conditions presented in each test scenario.

In some embodiments, the evaluation and/or measurement module 1150 may be further configured to implement advanced algorithms to interpret complex eye movement patterns and response data, translating raw tracking data into meaningful metrics for each visual function being assessed. In some embodiments, the evaluation and/or measurement module 1150 may be further configured to generate comprehensive reports detailing the results of visual function assessments, including quantitative measures of visual acuity, depth perception, motion detection, astigmatism, and night vision capabilities. In some embodiments, the evaluation and/or measurement module 1150 may be further configured to provide real-time feedback during testing sessions, allowing for dynamic adjustment of test parameters based on ongoing performance and response patterns. These features enable the system to conduct thorough, quantitative evaluations of various aspects of visual function based on eye movement data and/or user responses, supporting detailed analysis and measurement of visual capabilities within the VR environment.

Each of the above identified executable modules, applications, or sets of procedures may be stored in one or more of the previously mentioned memory devices and corresponds to a set of instructions for performing a function described above. The above identified modules or programs (i.e., sets of instructions) need not be implemented as separate software programs, procedures, or modules, and thus various subsets of these modules may be combined or otherwise rearranged in various embodiments. In some embodiments, the memory 1124 stores a subset of the modules and data structures identified above. Furthermore, in some embodiments, the memory 1124 stores additional modules or data structures not described above. Example details and/or operations of the modules, data structures, applications and/or procedures, are further described below, according to some embodiments.

Although FIG. 11A shows a computing device, FIG. 11A is intended more as a functional description of the various features that may be present rather than as a structural schematic of the embodiments described herein. In practice, and as recognized by those of ordinary skill in the art, items shown separately could be combined and some items could be separated.

### VR Light Sensitivity Testing System

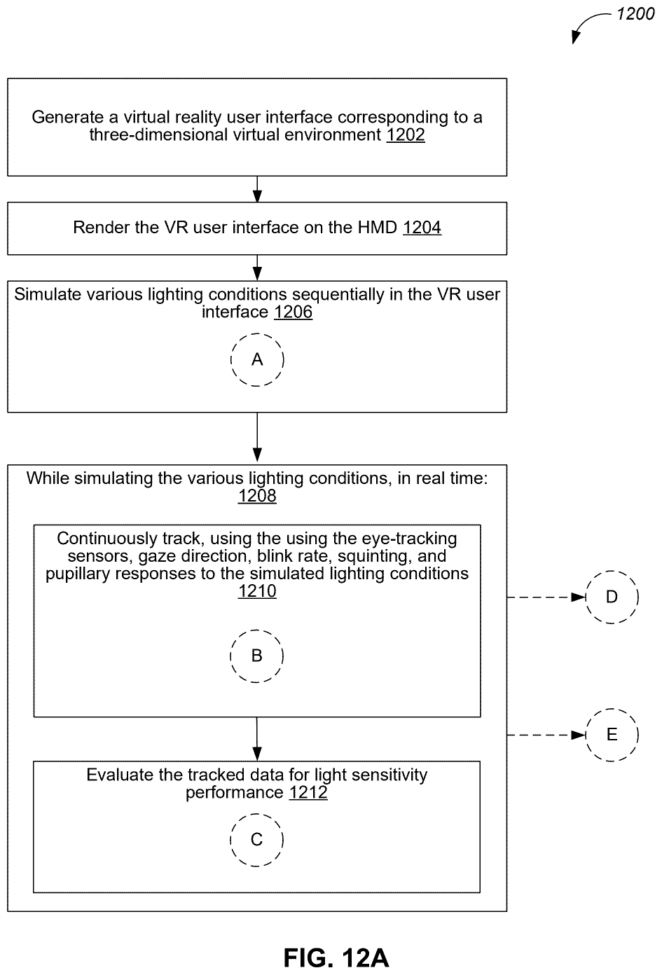

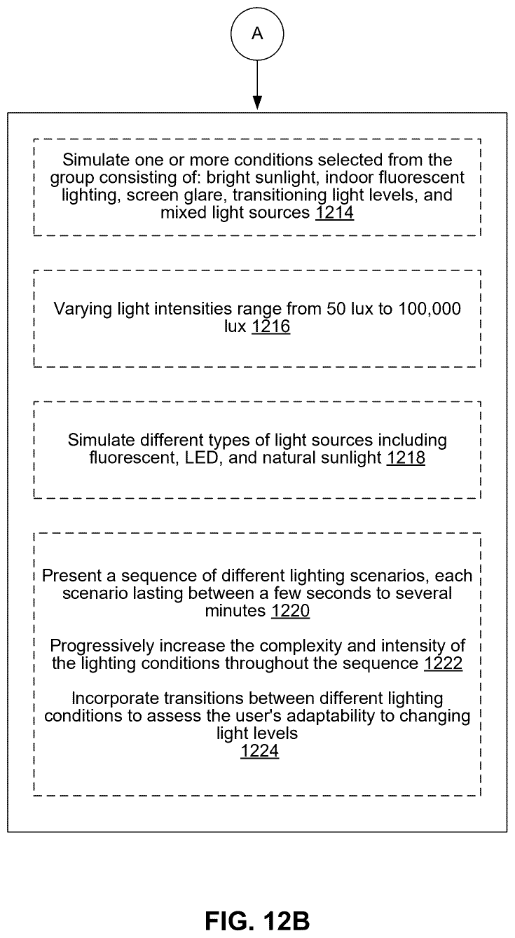

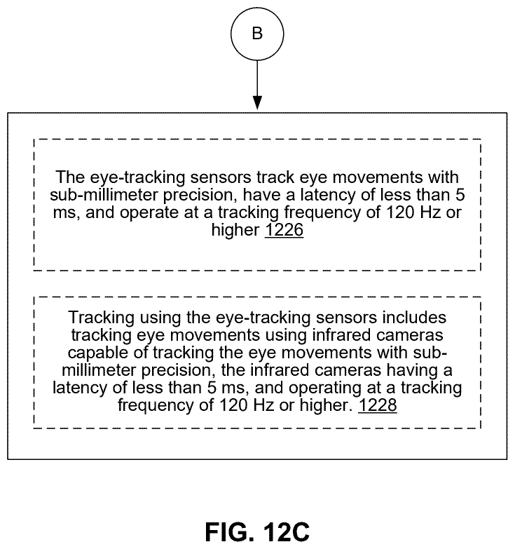

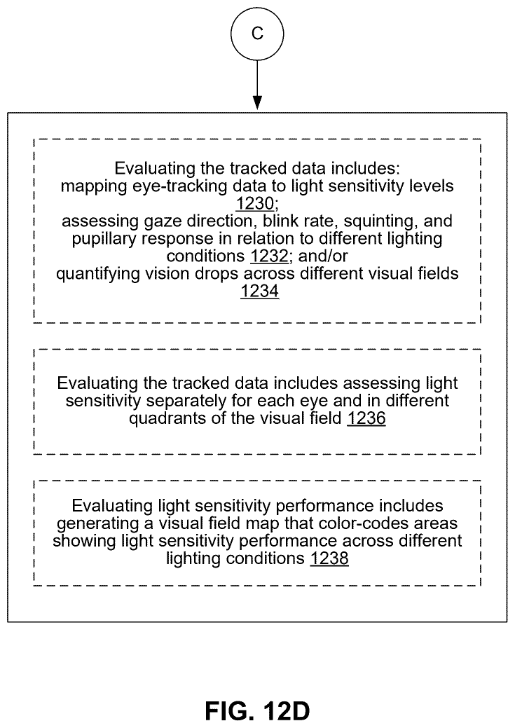

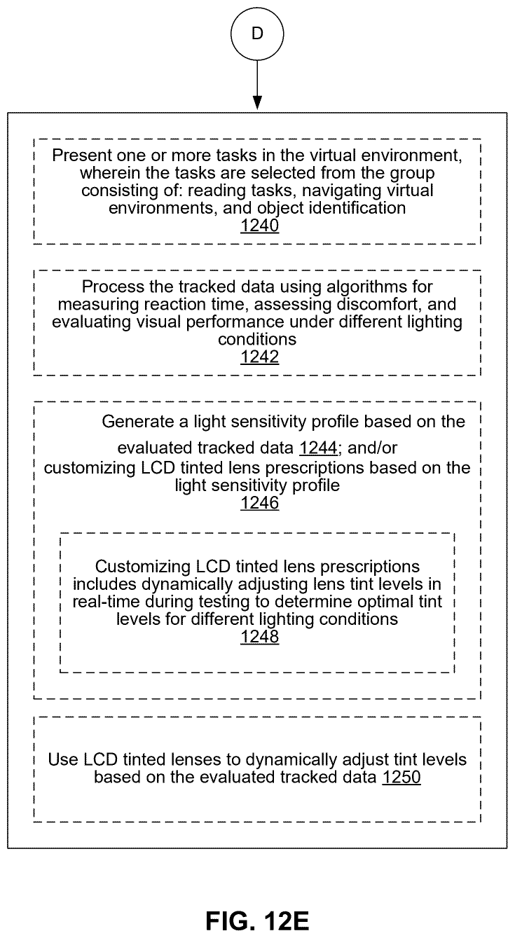

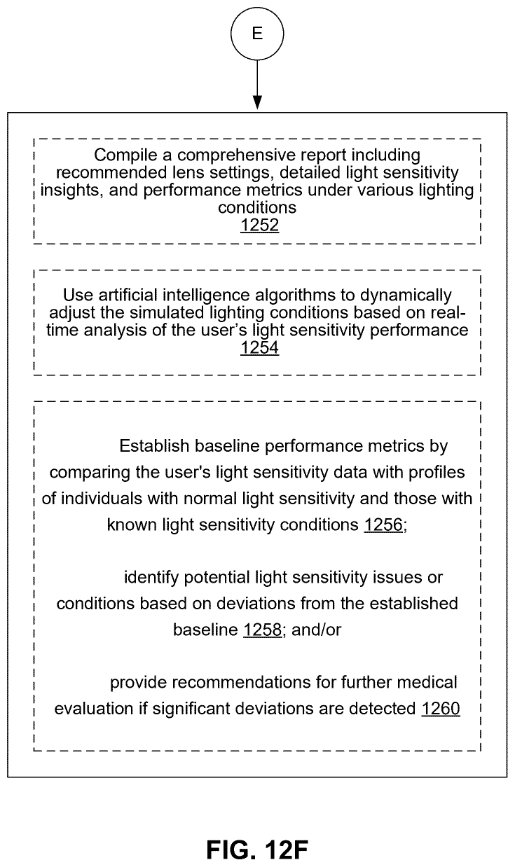

According to some embodiments, the vision test system 1100 described above is configured to implement a virtual vision test for evaluating list sensitivity and/or prescribing customized LCD tinted lenses. FIGS. 12A-12F show a flow diagram of an example process 1200 for implementing a virtual reality (VR) system for testing light sensitivity and prescribing customized LCD tinted lenses, according to some embodiments.

The computer device 140 (e.g., the computing device described above in reference to FIGS. 11A and 11B) generates (e.g., in step 1202) (e.g., using the UI module 1134) a virtual reality (VR) user interface (UI) corresponding to a three-dimensional virtual environment (e.g., an environment 1136). In some embodiments, game engines (e.g., platforms like Unity or Unreal Engine) are used to implement the UI and integrate it with the virtual environment. 3D modeling software can be used for creating assets that may be part of the UI in the photorealistic environment.

In some embodiments, this step is performed on a host computer, whereby the main processing unit (CPU) and graphics card (GPU) of the computer connected to a VR/AR headset handles much of the heavy lifting for generating and rendering the UI. This can be useful for tethered VR headsets that rely on a powerful PC for processing. In some embodiments, this step is performed on the headset itself. Standalone VR/AR headsets have onboard processors that can handle some or all of the UI generation and rendering. This on-device processing provides responsive, low-latency interactions. Cloud processing can also be used for some aspects of UI generation. For example, tasks requiring heavy computation might be offloaded to cloud servers and streamed to the headset. A combination of the above, with some elements pre-baked during development, some processed on a host PC, and some handled by the headset itself, can be used in some embodiments.

In some embodiments, the step of generating a VR UI corresponding to a photorealistic environment includes creating interactive visual elements that allow users to navigate and interact with a highly realistic 3D virtual world. Photorealistic virtual environment refers to a 3D digital space that looks and behaves as close to reality as possible. Advanced graphics, lighting, textures, and/or physics simulations can be used to create a highly detailed and lifelike virtual world. VR user interface is the set of visual elements, controls, and/or interaction methods that allow users to navigate, manipulate, and/or engage with the virtual environment. In VR, these interfaces are designed to be intuitive and immersive, often blending seamlessly with the virtual world.

Generating the interface may include generating UI elements that are both functional and visually consistent with the photorealistic environment. In various embodiments, this includes menus and buttons that appear to exist within the 3D space, gesture-based controls that feel natural in the virtual world, visual feedback that matches the aesthetic of the environment, and/or information displays that integrate with the surroundings. The computer device 140 creates an interface that enhances the user's sense of presence and immersion in the virtual world. This often means making UI elements that look and feel like they belong in the photorealistic environment, such as holographic displays or physical objects that the user can interact with using VR controllers or hand tracking.

Eye testing using photorealistic environments offers several advantages compared to traditional methods. Photorealistic environments provide a more accurate and comprehensive assessment of visual function. For example, photorealistic environments provide realistic simulation, mimic real-world conditions much more accurately than traditional eye charts or simple visual tests. This allows for a more accurate assessment of how well a person can see in everyday situations. These environments can change dynamically to simulate different lighting conditions, distances, and angles, providing a more comprehensive test of visual capabilities, including peripheral vision and depth perception.

Patients, especially children or those with attention difficulties, may find photorealistic environments more engaging than standard tests, leading to more reliable results as they are more likely to fully participate in the testing process. Traditional eye tests often focus on static images and high-contrast letters. Photorealistic environments, on the other hand, can be used to present complex, real-world visual tasks that can better assess functions like motion detection, contrast sensitivity, and/or color perception. Furthermore, the photorealistic environment can be customized to the specific needs or conditions of the patient, such as simulating the individual's workplace or home setting, providing a personalized and relevant assessment of their vision.

More complex and varied testing scenarios, which photorealistic environments can help simulate, can help in the early detection of visual problems that might not be apparent in traditional tests. This includes issues related to glare, night vision, and visual processing speeds. Advanced eye-tracking technology, specific examples of which are described herein, can be used in photorealistic environments to provide objective data on eye movements, fixation points, and response times, offering a more detailed analysis of visual function. For patients undergoing vision therapy or rehabilitation, photorealistic environments can provide a controlled yet realistic setting for practicing visual skills, making the training more effective and directly applicable to real-world tasks. Overall, eye testing using photorealistic environments described herein, represents a significant advancement in optometry and vision science, offering a richer, more detailed, and accurate assessment of visual health.

The computer device 140 renders (e.g., in step 1204) (e.g., using the rendering module 1138) the VR user interface on the HMD 1102. In some embodiments, photorealistic environments are displayed by leveraging various techniques and technologies described herein, according to some embodiments. Some embodiments use photogrammetry to create highly detailed 3D models from a set of photographs. By capturing real-world objects or environments from multiple angles, photogrammetry helps reconstruct their geometry and computer textures with a high degree of realism. In some embodiments, these models are then imported into the VR environment (sometimes referred to as the photorealistic environment or three-dimensional virtual environment).

Some embodiments provide 360-degree photography and videography. In some embodiments, VR devices display panoramic 360-degree photos and videos, which provide an immersive and photorealistic representation of real-world environments. In some embodiments, these are captured using specialized camera rigs or stitched together from multiple camera feeds. Some embodiments use real-time ray tracing. Modern graphics hardware and rendering techniques like real-time ray tracing help simulate the behavior of light in a physically accurate manner. By accurately modeling the interaction of light with materials, surfaces, and objects, ray tracing produces highly photorealistic images and environments in real-time. Some embodiments provide high-resolution textures and models. VR devices leverage high-resolution textures and detailed 3D models to create environments that closely resemble reality.

Some embodiments generate photorealistic environments using a combination of advanced rendering techniques and real-world data integration. High-resolution textures, captured through photogrammetry, may be mapped onto geometrically accurate 3D models. Global illumination algorithms, including ray tracing and radiosity, may be employed to simulate realistic lighting conditions. Physical-based rendering (PBR) materials may be used to accurately represent surface properties, such as reflectivity, roughness, and subsurface scattering. Dynamic elements, such as moving objects or changing weather conditions, may be simulated using particle systems and fluid dynamics algorithms. Some embodiments also incorporate real-time occlusion culling and level-of-detail (LOD) management to maintain high frame rates while preserving visual fidelity. To ensure consistency and repeatability, each photorealistic environment may be generated based on predefined parameters. These parameters may include lighting conditions, object placements, and/or atmospheric effects. In this way, some embodiments create controlled yet highly detailed environments that can be easily replicated or modified for different testing scenarios.

In some embodiments, the environments are created using techniques like photogrammetry, 3D scanning, or manually by artists and designers. Some embodiments use physically based rendering (PBR). PBR includes simulating the behavior of materials and their interactions with light based on real-world physics principles. By accurately modeling materials and their properties, such as roughness, metallic properties, and reflectance, PBR produces highly realistic visuals in VR environments. Some embodiments use image-based rendering, which includes using real-world photographs or video footage as the basis for rendering virtual environments. In some embodiments, by projecting and blending these images onto 3D geometry, a highly photorealistic environment is created. In some embodiments, VR devices capture real-world lighting information using techniques like light probes or environmental capture. This data can then be used to accurately simulate and recreate realistic lighting conditions within the virtual environment. By combining the techniques described herein and leveraging the latest advancements in graphics hardware and rendering algorithms, VR devices can provide highly immersive and photorealistic virtual experiences that closely resemble real-world environments.

Photorealistic environments used for eye testing can differ significantly from those used in VR games in several aspects, including design, functionality, and application. Photorealistic environments for eye testing are designed for precision, control, and repeatability to assess visual functions accurately, while those for VR games focus on creating immersive, interactive, and enjoyable experiences for entertainment. In contrast to VR games, eye testing requires clinical precision. Accordingly, some embodiments provide highly controlled and repeatable conditions for accurate diagnosis and assessment of visual functions. In some embodiments, specific scenarios are tailored to simulate real-world conditions that are relevant for visual testing, such as different lighting conditions, contrast levels, and visual tasks like reading or recognizing objects. Environments are kept consistent across tests to ensure reliable results. This includes controlled variations in visual stimuli to test specific aspects of vision.

Eye testing also requires precision tracking. Accordingly, some embodiments utilize high-precision eye-tracking to measure fine details of eye movements, fixations, and/or response times. Some embodiments collect accurate data for clinical analysis, including metrics, such as saccadic latency, fixation stability, and smooth pursuit accuracy. Some embodiments can include standardized visual tests, such as visual acuity tests, contrast sensitivity tests, and visual field tests.

In some embodiments, the photorealistic virtual environment prioritizes precision, control, repeatability and/or data collection over immersion, interaction, variety and/or user experience to assess visual functions accurately.

For example, a photorealistic environment for eye testing that includes a simulated driving environment can include a controlled simulation of driving conditions at night or in fog, designed to assess visual acuity, peripheral vision, and reaction times. The environment would include standardized visual stimuli, such as road signs, other vehicles, and pedestrians, which appear in predetermined patterns and intervals. For repeatability, each test is consistent, with the same conditions and stimuli presented in the same manner each time. This ensures that results can be reliably compared across different sessions or subjects. As another example, a photorealistic environment for eye testing that includes reading and office tasks can include a photorealistic simulation of an office environment with various reading tasks. This could include reading text on a computer screen, paper documents, and recognizing icons or objects on a cluttered desk.

For repeatability, text size, font, contrast, and lighting conditions are kept constant across tests. This allows precise measurement of reading speed, accuracy, and visual fatigue under standardized conditions. As yet another example, a supermarket simulation can include a virtual supermarket where patients are asked to locate and identify products on shelves. The environment would include standardized lighting, product placement, and visual clutter. For repeatability, the position and appearance of products remain the same in each test, ensuring that any changes in performance are due to the patient's vision and not variations in the environment. Eye testing environments prioritize controlled and repeatable conditions to ensure accurate measurement of visual functions instead of, or in addition to, focusing on creating immersive and interactive experiences that engage and entertain players. Eye testing environments are standardized to eliminate variables that could affect the results. A goal of eye testing environments, such as the ones described herein, is to collect precise data for clinical analysis, more than merely providing enjoyable user experience.

In the context of a photorealistic virtual environment designed for precise visual function assessment, qualities, such as precision, control, repeatability, and data collection, may be quantified or measured using the following methodologies. Precision may be quantified by measuring the variance in visual acuity scores or reaction times when the same stimuli are presented multiple times under identical conditions. A lower variance would indicate higher precision. Additionally, the spatial resolution of the visual stimuli may be quantified by the pixel density in the VR environment, where higher pixel density corresponds to higher precision in visual representation.

Control may be measured by assessing the fidelity of the virtual environment to real-world parameters. For instance, in a simulated driving environment, control may be quantified by how accurately the speed, direction, and lighting conditions match predefined standards. Metrics, such as frame rate stability, latency in rendering, and synchronization with real-world physics (e.g., gravity, friction) may serve as quantitative measures of control.

Repeatability may be quantified by the consistency of test results across multiple sessions. Statistical methods, such as calculating the intraclass correlation coefficient (ICC), may be used to measure the reliability of visual function assessments over time. A high ICC value may indicate that the VR environment consistently produces similar outcomes, highlighting strong repeatability. The effectiveness of data collection may be measured by the amount and quality of data points gathered during each session. This may include the resolution of eye-tracking data, the accuracy of response time measurements, and the granularity of physiological data (e.g., pupil dilation, heart rate). The completeness of data collection, indicated by minimal data loss or artifacts, may also be used.

In some embodiments, the photorealistic virtual environment corresponds to an environment selected from the group consisting of: urban streets, natural landscapes, indoor settings (e.g., living rooms, offices), and crowded public spaces (e.g., malls, transportation hubs). The system may define, store, and/or use scenarios with a level of detail and movement similar to busy intersections or trails by leveraging advanced computer graphics techniques and/or a robust database architecture.

For example, each environment, such as a busy intersection or a forest trail, may be defined by its unique set of visual and interactive elements. For a busy intersection, the system may include parameters, such as traffic density, pedestrian flow, vehicle speeds, traffic light cycles, and/or ambient noise levels. For a forest trail, the environment may include varying terrain textures, dynamic lighting based on time of day, and/or movement of flora and fauna.

Optionally, scenarios may be stored as modular data sets within the system's database. Each scenario may include 3D models, textures, lighting maps, and/or behavioral scripts that dictate how objects in the environment interact with the user.

For example, a busy intersection scenario may store detailed vehicle models, pedestrian avatars, and/or algorithms controlling their movement patterns. The storage system may be optimized for quick retrieval and modification, allowing scenarios to be adapted based on user requirements or testing protocols. The system may use these scenarios by dynamically loading them into the VR environment during testing.

The criteria for what constitute each environment can include various factors. For example, the criteria can include a Level of Detail (LOD). For busy intersections, for example, the LOD may include high-resolution textures for vehicles, road surfaces, and buildings, alongside complex shadowing and/or reflection effects. For trails, for example, the LOD may emphasize realistic foliage, ground textures, and/or subtle environmental movements like wind in the trees. The criteria can also include a movement complexity. In busy intersections, movement complexity may involve multiple objects (e.g., vehicles, pedestrians) moving at varying speeds and/or trajectories.

For trails, movement complexity may include the swaying of trees, shifting light through the canopy, and/or the user's interaction with uneven terrain; (iii) interactivity: The degree to which the user can interact with the environment may also define its complexity. In an intersection, users may respond to traffic signals, navigate around obstacles, and/or follow a vehicle's trajectory. In a trail scenario, interaction may include avoiding obstacles, tracking wildlife, and/or responding to changes in terrain.

In some embodiments, the photorealistic virtual environment corresponds (e.g., in step 1218) to an environment with varied lighting conditions (e.g., transitioning from daylight to twilight, using texture mapping techniques) and/or scenarios with a level of detail and movement similar to busy intersections or forest trails. Some embodiments use texture mapping, a technique used in computer graphics and 3D rendering, to add detailed surface information to 3D models. Texture mapping can be used for handling varying lighting conditions.

For example, light mapping includes pre-computing and storing lighting information in texture maps. Light maps capture the way light interacts with the geometry of a scene, including shadows, color bleeding, and other global illumination effects. By baking this information into texture maps, the lighting can be applied efficiently during real-time rendering without costly re-computation. Normal maps store per-pixel surface normal information in a texture. This allows the renderer to calculate accurate lighting by taking into account the high-frequency details captured in the normal map, even if the underlying geometry is relatively low-resolution. Normal mapping enhances the appearance of surface details and their interaction with light.

Ambient occlusion textures store pre-computed accessibility information, which approximates how exposed each surface point is to ambient lighting. This allows the renderer to apply physically based ambient shadowing effects without costly real-time calculations. Specular maps modulate the intensity and color of specular highlights on a surface. This allows for accurate representation of different material properties and their interactions with light sources in the scene. Self-shadowing and horizon mapping involve pre-computing and storing shadow information in textures, which can then be used to apply self-shadowing and atmospheric effects to objects in a consistent manner under varying lighting conditions. By combining these texture mapping techniques, in some embodiments, real-time rendering engines efficiently approximate complex lighting interactions, even in scenarios with dynamic lighting conditions. The pre-computed textures allow the renderer to produce realistic results while maintaining high performance, which is crucial for applications such as games, architectural visualization, and virtual reality.

In some embodiments, the VR user interface allows a user to navigate through virtual environments using natural head and eye movements, mimicking real-world interactions and responses. Natural head and eye movements in the context of a VR environment may be defined and/or measured using several parameters that reflect the typical behavior of these movements in real-world scenarios. For definition of natural movements, natural head movements may be characterized by the range, speed, and/or smoothness with which users typically move their heads when engaging with their environment. This may include nodding, turning the head left or right, tilting, and/or the combination of these movements during tasks, such as scanning a room or focusing on different objects in the VR environment.

Natural eye movements may be defined by saccades (quick jumps of the eye between fixation points), fixations (periods where the eyes are stationary and focused on a single point), and/or smooth pursuit (the eye's ability to track a moving object). The parameters may include saccadic velocity, fixation duration, and/or the accuracy of smooth pursuit. Head movements may be measured using gyroscopes and accelerometers embedded in the VR headset. The system may record the angular velocity and acceleration of the head in three axes (pitch, yaw, and roll) and/or compare these metrics against established norms for natural head movements. Eye movements may be measured using infrared eye-tracking technology that monitors the position and movement of the eyes within the VR headset. The system may capture data on saccadic movements, including their amplitude, velocity, and frequency, as well as fixation stability and duration. Smooth pursuit may be measured by tracking the eye's ability to follow a moving target with minimal lag or deviation.

Referring back to FIG. 12A, the computer device 140 simulates (e.g., in step 1206) (e.g., using the simulation module 1140) various lighting conditions (e.g., the scenarios 1142) sequentially in the VR user interface. Referring to FIG. 12B, in some embodiments, the computer device 140 simulates the various lighting conditions by simulating (e.g., in step 1214) one or more conditions selected from the group consisting of: bright sunlight, indoor fluorescent lighting, screen glare, transitioning light levels, and mixed light sources. In some embodiments, simulating various lighting conditions includes (e.g., in step 1216) varying light intensities ranging from 50 lux to 100,000 lux.

In some embodiments, simulating various lighting conditions includes simulating (e.g., in step 1218) different types of light sources including fluorescent, LED, and natural sunlight. In some embodiments, simulating various lighting conditions includes: presenting (e.g., in step 1220) a sequence of different lighting scenarios, each scenario lasting between a few seconds to several minutes; progressively increasing (e.g., in step 1222) the complexity and intensity of the lighting conditions throughout the sequence; and incorporating (e.g., in step 1224) transitions between different lighting conditions to assess the user's adaptability to changing light levels.

The computer device 140 may provide precision and consistency of visual stimuli. Several advanced techniques and technologies may be integrated for ensuring precision, consistency, and accurate tracking in a VR environment designed for visual assessments requires. For precision, high-resolution displays, for example using VR headsets with high pixel density and low sub-pixel variance, may help ensure that visual stimuli are presented with maximum clarity and detail. This may reduce the chances of aliasing or blurring, which can affect the accuracy of visual tests. Sub-millimeter accuracy tracking using advanced tracking systems, such as those employing multiple cameras or external sensors, may help ensure that head and eye movements are captured with sub-millimeter accuracy.

This level of precision may be used for detecting even the smallest deviations in eye movement, which can be indicative of underlying visual impairments. For consistency, standardized scenarios may be used. For example, all visual stimuli and scenarios within the VR environment may be standardized, meaning that each test presents the same conditions (lighting, object placement, timing) regardless of when or where the test is conducted. This consistency may be used for comparing results across different sessions or subjects. Maintaining a high and stable frame rate (e.g., 90 FPS or higher) may help ensure that the VR environment remains fluid and responsive, preventing motion blur or jitter, which could introduce inconsistencies in test results.

In the context of a VR environment used for visual function assessments, the minimum refresh rate that may be considered real-time is generally 90 Hz. This is because a refresh rate of 90 Hz or higher helps ensure smooth, fluid motion and to reduce motion sickness, which can occur at lower refresh rates. A 90 Hz refresh rate means that the display updates 90 times per second. This is useful for creating a seamless and immersive experience, especially when the user is interacting with dynamic environments that require real-time responses. A lower refresh rate may introduce latency, leading to visual artifacts or lag, which could compromise the accuracy of the visual tests.

Referring back to FIG. 12A, the computer device 140, while simulating (e.g., in step 1208) the various lighting conditions, in real time, continuously tracks (e.g., in step 1210) (e.g., using the tracking module 1144), using the eye-tracking sensors, gaze direction, blink rate, squinting, and/or pupillary responses (e.g., the eye movements 1146) to the simulated lighting conditions. Referring to FIG. 12C, in some embodiments, the eye-tracking sensors track (e.g., in step 1226) eye movements with sub-millimeter precision, have a latency of less than 5 ms, and operate at a tracking frequency of 120 Hz or higher. In some embodiments, tracking using the eye-tracking sensors includes (e.g., in step 1228) tracking eye movements using infrared cameras capable of tracking the eye movements with sub-millimeter precision, the infrared cameras having a latency of less than 5 ms and operating at a tracking frequency of 120 Hz or higher.

Calibrated eye-tracking systems may be used. For example, eye-tracking systems may be calibrated for each user to account for individual differences in eye physiology, such as interpupillary distance (IPD) and eye dominance. Calibration may help ensure that the system accurately tracks the user's gaze direction, fixation points, and saccadic movements. In environments where high accuracy is paramount, redundant tracking systems (e.g., combining inside-out tracking with external cameras) may be employed. This redundancy may help cross-verify data and correct any potential inaccuracies caused by a single tracking method. The VR system may continuously monitor the tracking data in real time to detect and/or correct any anomalies. For example, if the system detects a sudden, unrealistic jump in eye movement, the system may prompt a recalibration or discard the aberrant data to maintain the accuracy of the test results.

Referring back to FIG. 12A, the computer device 140, while simulating the various lighting conditions, in real time, evaluates (e.g., in step 1212) (e.g., using the evaluation/measurement module 1150) the tracked data for light sensitivity performance (e.g., the light sensitivity performance 1152). Referring to FIG. 12D, in some embodiments, the computer device 140 evaluates the tracked data by mapping (e.g., in step 1230) eye-tracking data to light sensitivity levels, assessing (e.g., in step 1232) gaze direction, blink rate, squinting, and pupillary response in relation to different lighting conditions, and/or quantifying (e.g., in step 1234) vision drops across different visual fields. In some embodiments, the computer device 140 evaluates the tracked data by assessing (e.g., in step 1236) light sensitivity separately for each eye and in different quadrants of the visual field. In some embodiments, the computer device 140 evaluates light sensitivity performance by generating (e.g., in step 1238) a visual field map that color-codes areas showing light sensitivity performance across different lighting conditions.

Referring to FIG. 12E, in some embodiments, the computer device 140 presents (e.g., in step 1240) one or more tasks in the virtual environment. The tasks may be selected from the group consisting of: reading tasks, navigating virtual environments, and object identification. In some embodiments, the computer device 140 processes (e.g., in step 1242) the tracked data using algorithms for measuring reaction time, assessing discomfort, and evaluating visual performance under different lighting conditions. In some embodiments, the computer device 140 generates (e.g., in step 1244) a light sensitivity profile based on the evaluated tracked data, and/or customizes (e.g., in step 1246) LCD tinted lens prescriptions based on the light sensitivity profile. In some embodiments, customizing LCD tinted lens prescriptions includes dynamically adjusting (e.g., in step 1248) lens tint levels in real-time during testing to determine optimal tint levels for different lighting conditions. In some embodiments, the computer device 140 uses (e.g., in step 1250) LCD tinted lenses to dynamically adjust tint levels based on the evaluated tracked data.

Some embodiments customize LCD tinted lenses using a multi-step process, which may translate light sensitivity data into optimal tint configurations. Initially, for instance, the system may analyze pupillary responses, blink rates, and/or performance metrics across various lighting conditions to create a detailed light sensitivity profile. This profile may be then mapped onto a multidimensional color space, where each dimension may correspond to a specific tint parameter (e.g., intensity, hue, opacity). Some embodiments use optimization algorithms, to iteratively adjust these tint parameters in real-time during testing. Each adjustment may be immediately simulated in the virtual environment, allowing for instant feedback on its effectiveness. Some embodiments use a gradient descent method to converge on an optimal tint configuration that, for example, maximizes visual comfort and performance across all tested lighting conditions. This dynamic adjustment process helps ensure that the final tint prescription is tailored not only to a user's overall sensitivity but also to their specific responses, for example, in different real-world lighting scenarios.

Referring next to FIG. 12F, in some embodiments, the computer device 140 compiles (e.g., in step 1252) a comprehensive report including recommended lens settings, detailed light sensitivity insights, and performance metrics under various lighting conditions. In some embodiments, the computer device 140 uses (e.g., in step 1254) artificial intelligence algorithms to dynamically adjust the simulated lighting conditions based on real-time analysis of the user's light sensitivity performance. In some embodiments, the artificial intelligence (AI) algorithms employed for dynamically adjusting simulated lighting conditions use a combination of reinforcement learning and adaptive neural networks. In some embodiments, the system continuously analyzes, for instance, the user's light sensitivity performance metrics, including pupillary responses, blink rates, and/or task completion accuracy. This real-time data may be input to a deep learning model, which may predict optimal lighting adjustments to challenge the user's visual system while avoiding excessive discomfort. In some embodiments, the AI model is initially trained on a large dataset of light sensitivity profiles and corresponding optimal lighting conditions. During each test session, the model may fine-tune its predictions based on a user's responses, for example, employing transfer learning techniques to rapidly adapt to unique sensitivity patterns. The system may use a multi-armed bandit algorithm, for example, to balance exploration of new lighting conditions with exploitation of known effective settings, ensuring a comprehensive yet efficient testing process.

In some embodiments, the computer device 140 establishes (e.g., in step 1256) baseline performance metrics by comparing the user's light sensitivity data with profiles of individuals with normal light sensitivity and those with known light sensitivity conditions, identifies (e.g., in step 1258) potential light sensitivity issues or conditions based on deviations from the established baseline, and/or provides (e.g., in step 1260) recommendations for further medical evaluation if significant deviations are detected.

### VR Lens Tint Recommendation System

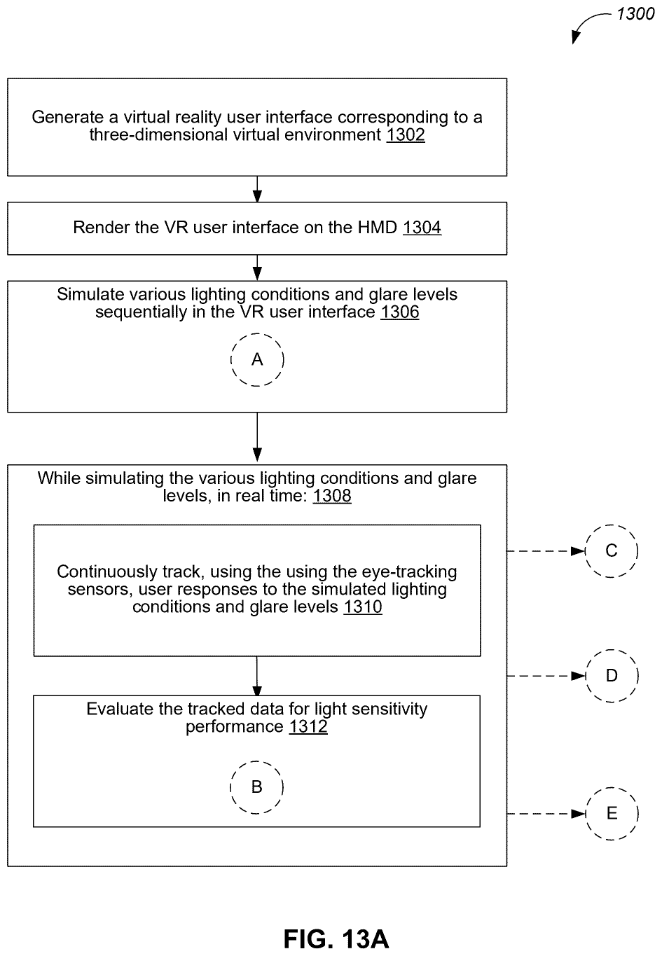

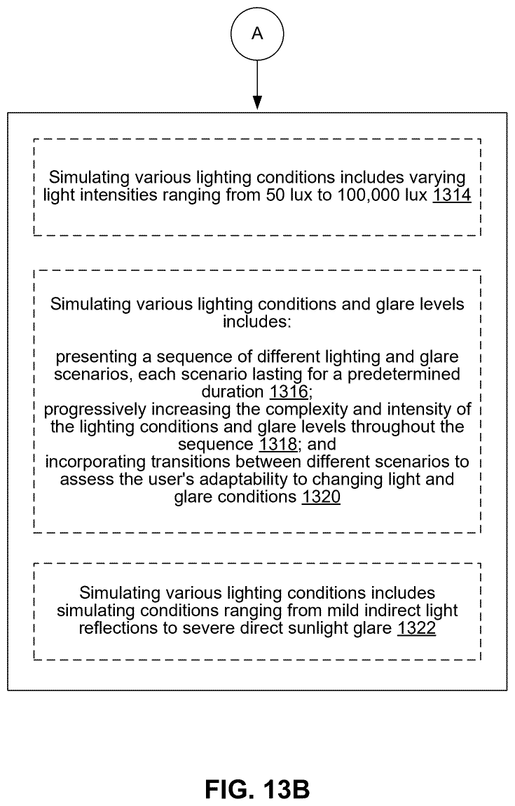

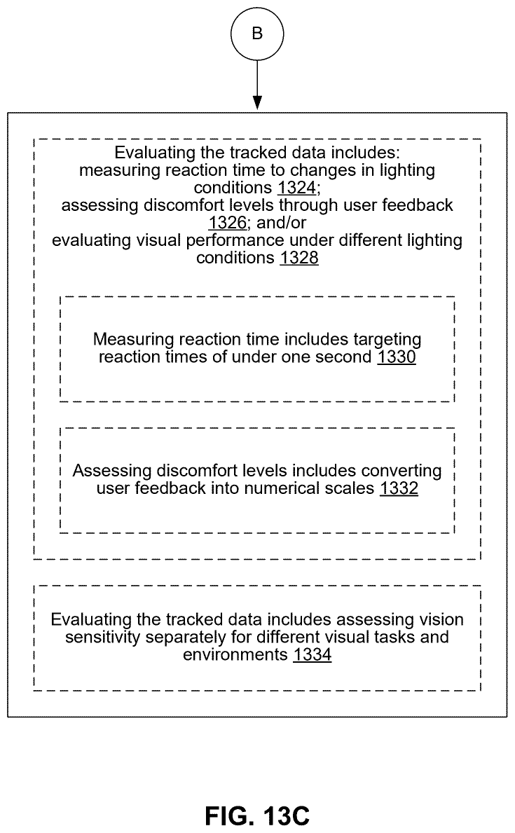

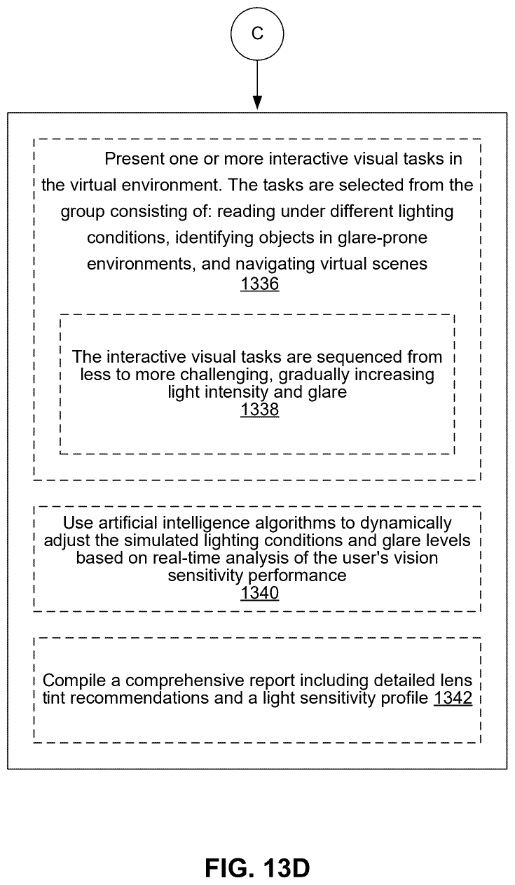

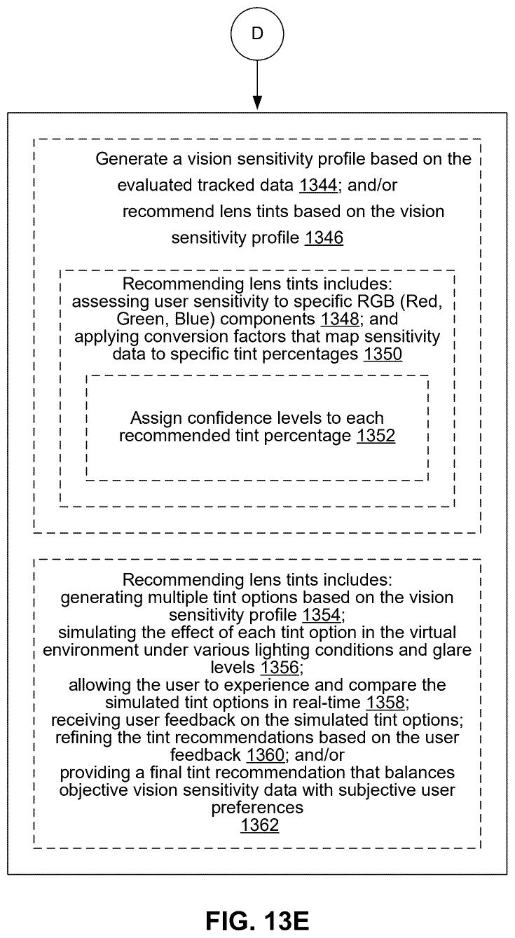

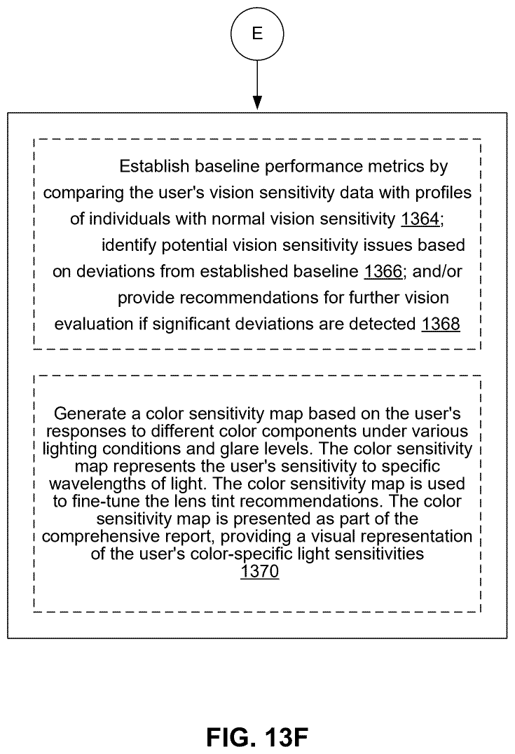

According to some embodiments, the vision test system 1100 described above is configured to implement a method for recommending lens tints through an interactive vision sensitivity test. FIGS. 13A-13F show a flow diagram of an example process 1300 for recommending lens tints through an interactive vision sensitivity test, according to some embodiments.

The computer device 140 (e.g., the computing device described above in reference to FIGS. 11A and 11B) generates (e.g., in step 1302) (e.g., using the UI module 1134) a VR user interface corresponding to a three-dimensional virtual environment.

The computer device 140 also renders (e.g., in step 1304) (e.g., using the rendering module 1138) the VR user interface on the HMD 312A. Example details of the three-dimensional virtual environment and rendering the VR user interface are described above in reference to FIG. 12B, according to some embodiments.

The computer device 140 also simulates (e.g., in step 1302) (e.g., using the simulation module 1140) various lighting conditions and glare levels sequentially (e.g., the test sequences 1142) in the VR user interface 1204.

Referring next to FIG. 13B, in some embodiments, the computer device 140 simulates various lighting conditions by varying (e.g., in step 1314) light intensities ranging from 50 lux to 100,000 lux. In some embodiments, the computer device 140 simulates various lighting conditions and glare levels by, for example, presenting (e.g., in step 1316) a sequence of different lighting and glare scenarios, each scenario lasting for a predetermined duration, progressively increasing (e.g., in step 1318) the complexity and intensity of the lighting conditions and glare levels throughout the sequence, and incorporating (e.g., in step 1320) transitions between different scenarios to assess the user's adaptability to changing light and glare conditions. In some embodiments, the computer device 140 simulates various glare levels by simulating (e.g., in step 1322) conditions ranging from mild indirect light reflections to severe direct sunlight glare.

Referring back to FIG. 13A, the method also includes continuously tracking (e.g., in step 1310) (e.g., using the tracking module 1144), using the eye-tracking sensors, user responses to the simulated lighting conditions and glare levels.

The method also includes evaluating (e.g., in step 1312) (e.g., using the evaluation/measurement module 1150) the tracked data for vision sensitivity performance (e.g., the visual sensitivity performance 1154). Referring next to FIG. 13C, in some embodiments, the computer device 140 evaluates the tracked data by measuring (e.g., in step 1324) reaction time to changes in lighting conditions, assessing (e.g., in step 1326) discomfort levels through user feedback, and/or evaluating (e.g., in step 1328) visual performance under different lighting conditions. In some embodiments, the computer device 140 measures reaction time by targeting (e.g., in step 1330) reaction times of under one second. In some embodiments, the computer device 140 assesses discomfort levels by converting (e.g., in step 1332) user feedback into numerical scales. In some embodiments, the computer device 140 evaluates the tracked data by assessing (e.g., in step 1334) vision sensitivity separately for different visual tasks and environments.

Referring next to FIG. 13D, in some embodiments, the computer device 140 presents (e.g., in step 1336) one or more interactive visual tasks in the virtual environment. The tasks may be selected from the group consisting of: reading under different lighting conditions, identifying objects in glare-prone environments, and navigating virtual scenes. I some embodiments, the interactive visual tasks are sequenced (e.g., in step 1338) from less to more challenging, gradually increasing light intensity and glare. In some embodiments, the computer device 140 uses (e.g., in step 1340) artificial intelligence algorithms to dynamically adjust the simulated lighting conditions and glare levels based on real-time analysis of the user's vision sensitivity performance. In some embodiments, the computer device 140 compiles (e.g., in step 1342) a comprehensive report including detailed lens tint recommendations and a light sensitivity profile.

Referring next to FIG. 13E, in some embodiments, the computer device 140 also generates (e.g., in step 1344) a vision sensitivity profile based on the evaluated tracked data, and recommends (e.g., in step 1346) lens tints based on the vision sensitivity profile. In some embodiments, recommending lens tints includes assessing (e.g., in step 1348) user sensitivity to specific RGB (Red, Green, Blue) components, and applying (e.g., in step 1350) conversion factors that map sensitivity data to specific tint percentages. In some embodiments, the computer device 140 further assigns (e.g., in step 1352) confidence levels to each recommended tint percentage.

In some embodiments, the computer device 140 also recommends lens tints by generating (e.g., in step 1354) multiple tint options based on the vision sensitivity profile, simulating (e.g., in step 1356) the effect of each tint option in the virtual environment under various lighting conditions and glare levels, allowing (e.g., in step 1358) the user to experience and compare the simulated tint options in real-time, receiving (e.g., in step 1360) user feedback on the simulated tint options, refining the tint recommendations based on the user feedback, and/or providing (e.g., in step 1362) a final tint recommendation that balances objective vision sensitivity data with subjective user preferences.

Referring next to FIG. 13F, in some embodiments, the computer device 140 also establishes (e.g., in step 1364) baseline performance metrics by comparing the user's vision sensitivity data with profiles of individuals with normal vision sensitivity, identifies (e.g., in step 1366) potential vision sensitivity issues based on deviations from the established baseline, and/or provides (e.g., in step 1368) recommendations for further vision evaluation if significant deviations are detected. In some embodiments, the computer device 140 also generates (e.g., in step 1370) a color sensitivity map based on the user's responses to different color components under various lighting conditions and glare levels. The color sensitivity map may represent the user's sensitivity to specific wavelengths of light. The color sensitivity map may be used to fine-tune the lens tint recommendations. The color sensitivity map may be presented as part of the comprehensive report, providing a visual representation of the user's color-specific light sensitivities.

### VR-Enabled Color Blindness Test Using Color-Coded Challenges and Puzzles

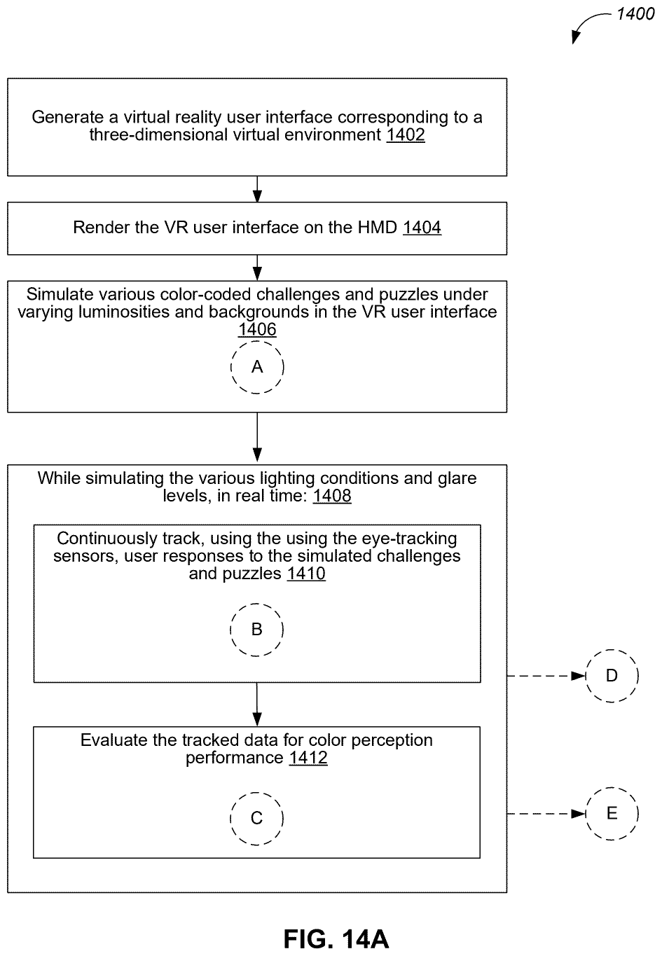

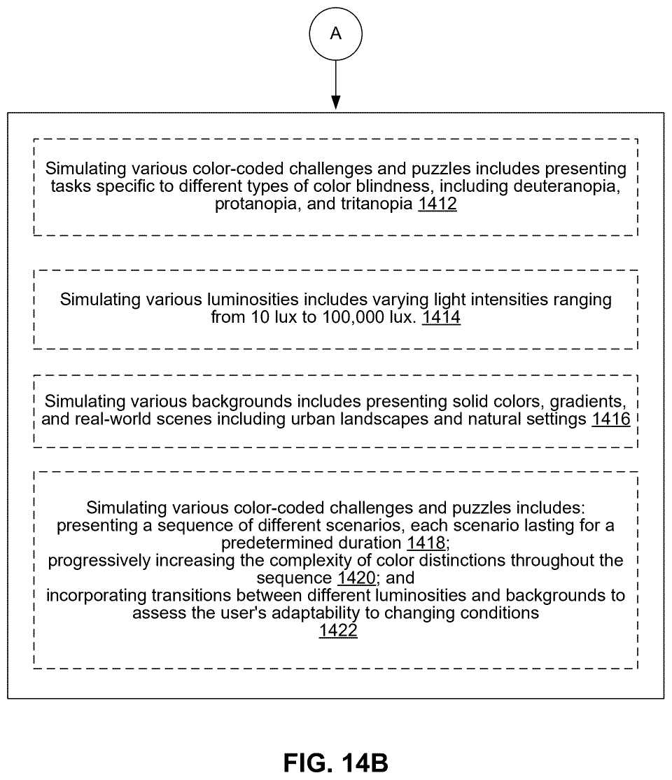

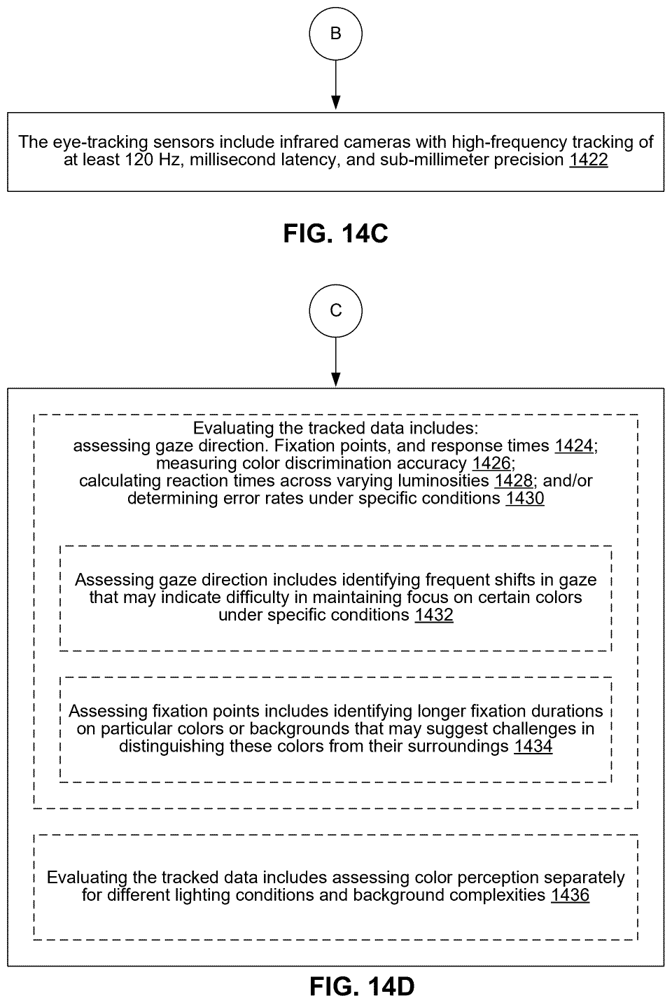

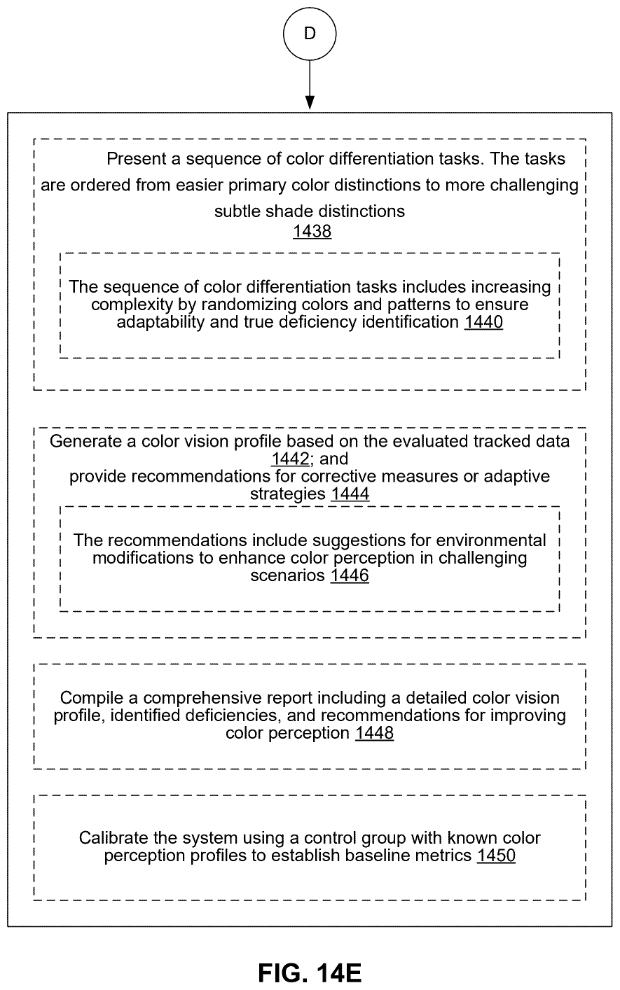

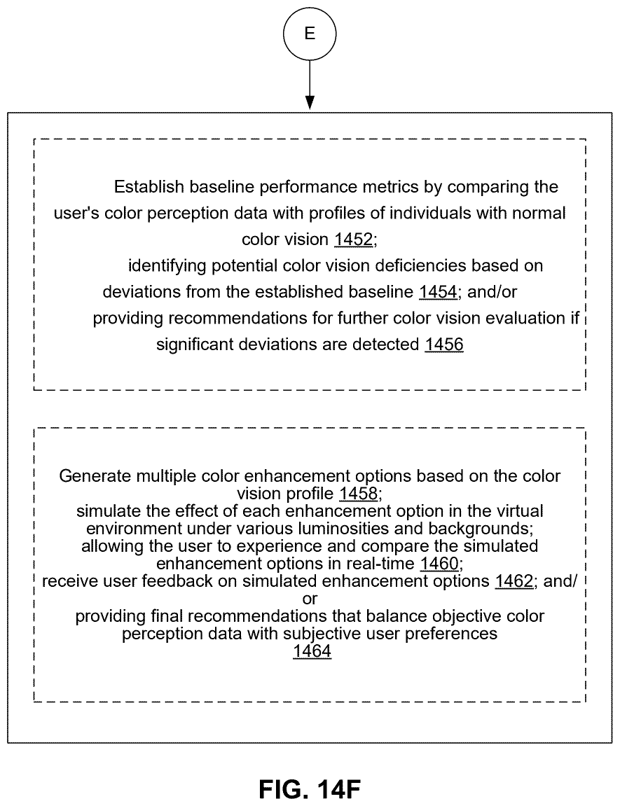

According to some embodiments, the vision test system 1100 described above is configured to implement a virtual eye test for evaluating color perception. FIGS. 14A-14F show a flow diagram of an example process 1400 for implementing a virtual eye test for color blindness using color-coded challenges and/or puzzles, according to some embodiments.

The computer device 140 (e.g., the computing device described above in reference to FIGS. 11A and 11B) generates (e.g., in step 1402) (e.g., using the UI module 1134) a VR user interface corresponding to a three-dimensional virtual environment (e.g., the environment 1136).

The computer device 140 also renders (e.g., in step 1404) (e.g., using the rendering module 1138) the VR user interface on the HMD 312A. Example details of the three-dimensional virtual environment and rendering the VR user interface are described above in reference to FIG. 12B, according to some embodiments.

The computer device 140 also simulates (e.g., in step 1406) (e.g., using the simulation module 1140) various color-coded challenges and/or puzzles under varying luminosities and backgrounds (e.g., the scenario 1142) in the VR user interface. Referring to FIG. 14B, in some embodiments, simulating various color-coded challenges and/or puzzles under varying luminosities and backgrounds includes presenting (e.g., in step 1412) tasks specific to different types of color blindness, including deuteranopia, protanopia, and tritanopia. In some embodiments, simulating various luminosities includes varying (e.g., in step 1414) light intensities ranging from 10 lux to 100,000 lux. In some embodiments, simulating various backgrounds includes presenting (e.g., in step 1416) solid colors, gradients, and real-world scenes including urban landscapes and natural settings. in some embodiments, simulating various color-coded challenges and puzzles includes presenting (e.g., in step 1418) a sequence of different scenarios, each scenario lasting for a predetermined duration, progressively increasing (e.g., in step 1420) the complexity of color distinctions throughout the sequence, and/or incorporating (e.g., in step 1422) transitions between different luminosities and backgrounds to assess the user's adaptability to changing conditions.

Referring back to FIG. 14A, while simulating the various lighting conditions and glare levels, in real time (e.g., in step 1408), the computer device 140 also continuously tracks (e.g., in step 1410) (e.g., using the tracking module 1144), the eye-tracking sensors, user responses (e.g., the responses 1148) to the simulated challenges and puzzles. Referring to FIG. 14C, in some embodiments, the eye-tracking sensors include (e.g., in step 1422) infrared cameras with high-frequency tracking of at least 120 Hz, millisecond latency, and/or sub-millimeter precision.

Referring again to FIG. 14A, the computer device 140 also evaluates (e.g., in step 1412) the tracked data for color perception performance (e.g., the color perception performance 1158). Referring to FIG. 14D, in some embodiments, the computer device 140 evaluates the tracked data by assessing (e.g., in step 1424) gaze direction, fixation points, and response times, measuring (e.g., in step 1426) color discrimination accuracy, calculating (e.g., in step 1428) reaction times across varying luminosities, and/or determining (e.g., in step 1430) error rates under specific conditions. In some embodiments, the computer device 140 assesses gaze direction by identifying (e.g., in step 1432) frequent shifts in gaze that may indicate difficulty in maintaining focus on certain colors under specific conditions. In some embodiments, the computer device 140 assesses fixation points by identifying (e.g., in step 1434) longer fixation durations on particular colors or backgrounds that may suggest challenges in distinguishing these colors from their surroundings. In some embodiments, the computer device 140 evaluates the tracked data by assessing (e.g., in step 1436) color perception separately for different lighting conditions and background complexities.

Referring next to FIG. 14E, in some embodiments, the computer device 140 also presents (e.g., in step 1438) a sequence of color differentiation tasks. The tasks are ordered from easier primary color distinctions to more challenging subtle shade distinctions. In some embodiments, the sequence of color differentiation tasks includes increasing (e.g., in step 1440) complexity by randomizing colors and patterns to ensure adaptability and true deficiency identification. In some embodiments, the computer device 140 also generates (e.g., in step 1442) a color vision profile based on the evaluated tracked data, and/or provides (e.g., in step 1444) recommendations for corrective measures or adaptive strategies. In some embodiments, the recommendations include (e.g., in step 1446) suggestions for environmental modifications to enhance color perception in challenging scenarios. In some embodiments, the computer device 140 also generates (e.g., in step 1448) a comprehensive report including a detailed color vision profile, identified deficiencies, and recommendations for improving color perception. In some embodiments, the computer device 140 also calibrates (e.g., in step 1450) the system using a control group with known color perception profiles to establish baseline metrics.

Referring next to FIG. 14F, in some embodiments, the computer device 140 also establishes (e.g., in step 1452) baseline performance metrics by comparing the user's color perception data with profiles of individuals with normal color vision, identifies (e.g., in step 1454) potential color vision deficiencies based on deviations from the established baseline, and/or provides (e.g., in step 1456) recommendations for further color vision evaluation if significant deviations are detected. In some embodiments, the computer device 140 also generates (e.g., in step 1456) multiple color enhancement options based on the color vision profile, simulates (e.g., in step 1458) the effect of each enhancement option in the virtual environment under various luminosities and backgrounds, allows (e.g., in step 1460) the user to experience and compare the simulated enhancement options in real-time, receives (e.g., in step 1462) user feedback on the simulated enhancement options, and/or provides (e.g., in step 1464) final recommendations that balance objective color perception data with subjective user preferences.

In some embodiments, color sensitivity testing in the virtual reality environment is implemented using a multifaceted approach that leverages the unique capabilities of VR technology. The system, for example, may generate a wide spectrum of color stimuli, precisely controlled in terms of hue, saturation, and/or brightness. These stimuli may be presented in various forms, including simple color patches, complex patterns, and real-world object simulations. The testing protocol may include adaptive psychophysical methods, such as the staircase procedure and/or forced-choice paradigms, to efficiently determine color discrimination thresholds. The system may, for example, vary the color differences between stimuli dynamically based on responses, converging on sensitivity measurements for different regions of a color space. Additionally, the VR environment may allow for the simulation of various lighting conditions and backgrounds, enabling the assessment of color constancy and simultaneous color contrast effects. In this way, the combination of precise stimulus control, adaptive testing methods, and/or realistic environmental simulations in VR helps provide a comprehensive evaluation of color sensitivity that surpasses traditional clinical tests in both accuracy and ecological validity.

### Evaluating Color Perception Under Varying Luminosities and Backgrounds in VR

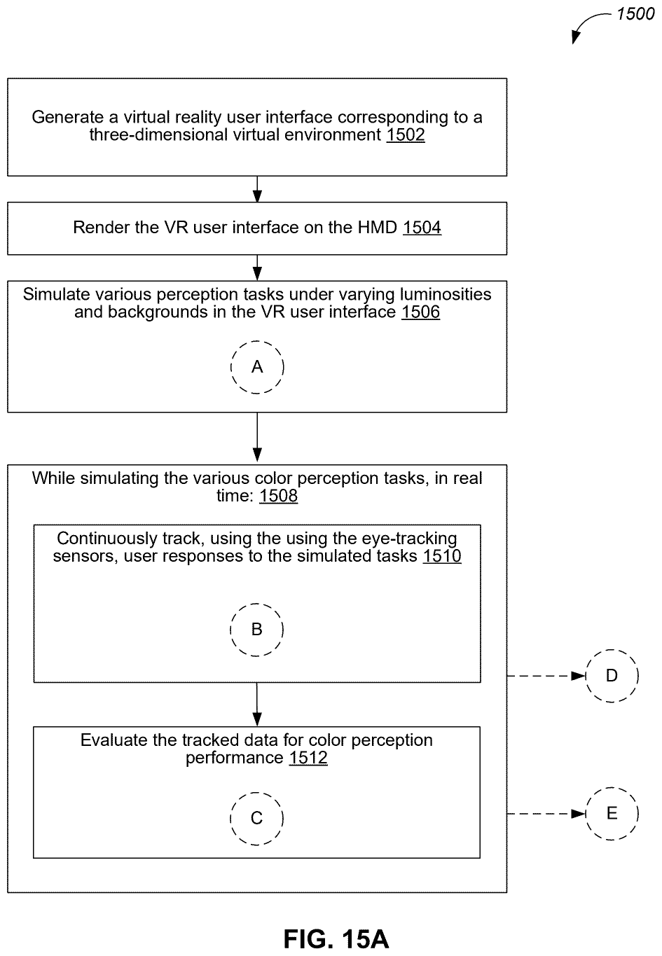

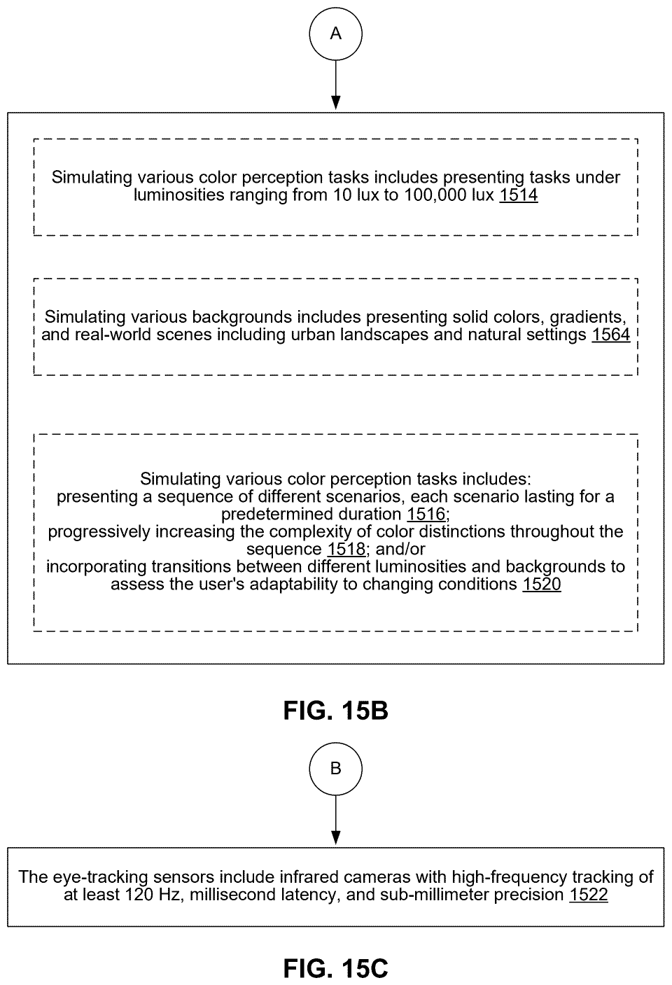

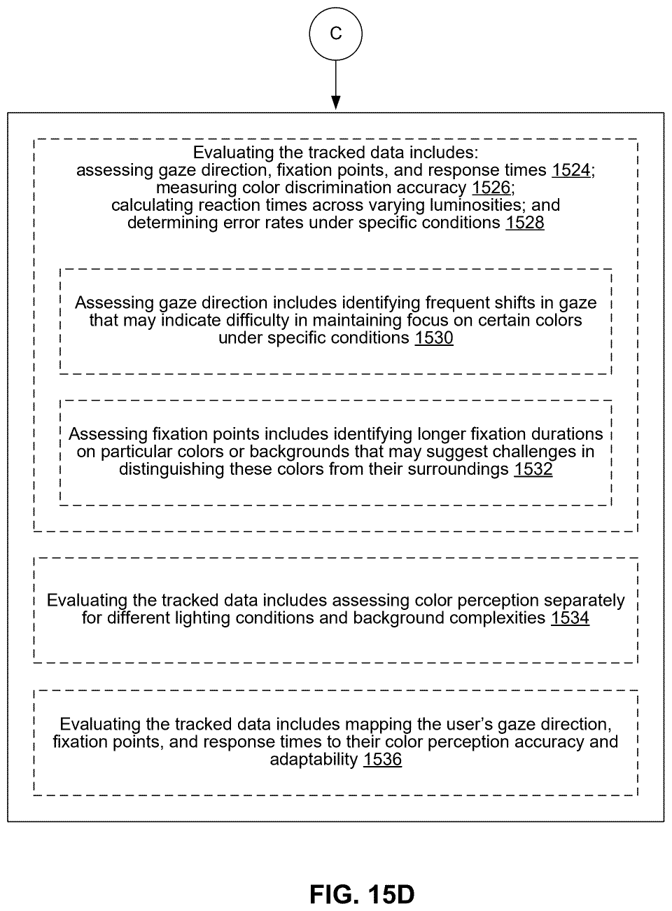

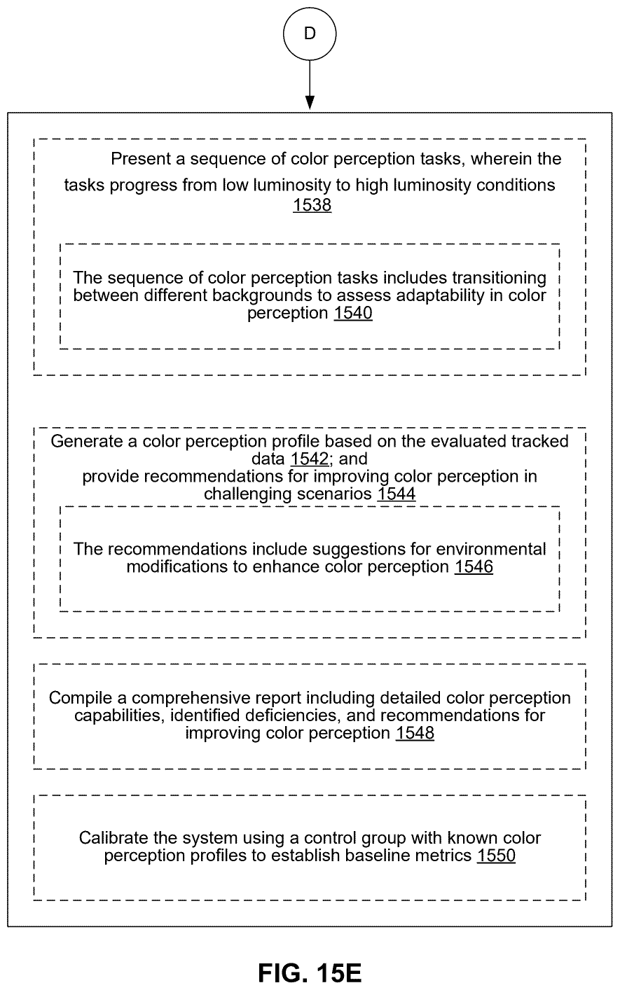

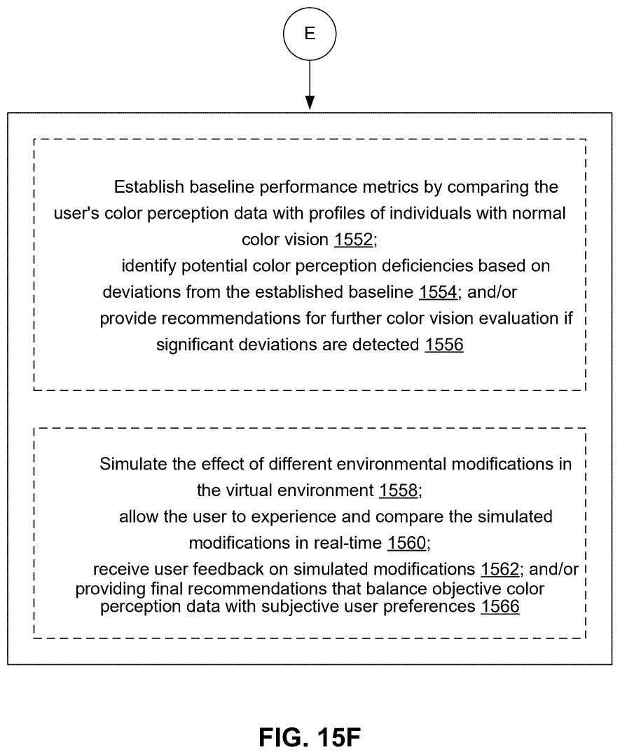

According to some embodiments, the vision test system 1100 described above is configured to evaluate color perception under varying luminosities and backgrounds. FIGS. 15A-15F show a flow diagram of an example process 1500 for implementing a virtual eye test for evaluating color perception under varying luminosities and backgrounds, according to some embodiments.

The computer device 140 (e.g., the computing device described above in reference to FIGS. 11A and 11B) generates (e.g., in step 1502) (e.g., using the UI module 1134) a VR user interface corresponding to a three-dimensional virtual environment (e.g., the environment 1136).

Referring back to FIG. 15A, the computer device 140 also renders (e.g., in step 1504) (e.g., using the rendering module 1138) the VR user interface on the HMD 312A. Example details of the three-dimensional virtual environment and rendering the VR user interface are described above in reference to FIG. 12B, according to some embodiments.

The computer device 140 also simulates (e.g., in step 1506) (e.g., using the simulation module 1140) various color perception tasks under varying luminosities and backgrounds (e.g., the scenarios 1142) in the VR user interface 1404. Referring to FIG. 15B, in some embodiments, the computer device 140 simulates various color perception tasks by presenting (e.g., in step 1514) tasks under luminosities ranging from 10 lux to 100,000 lux. In some embodiments, the computer device 140 simulates various backgrounds by presenting (e.g., in step 1564) solid colors, gradients, and real-world scenes including urban landscapes and natural settings. In some embodiments, the computer device 140 simulates various color perception tasks by presenting (e.g., in step 1516) a sequence of different scenarios, each scenario lasting for a predetermined duration, progressively increasing (e.g., in step 1518) the complexity of color distinctions throughout the sequence, and/or incorporating (e.g., in step 1520) transitions between different luminosities and backgrounds to assess the user's adaptability to changing conditions Referring back to FIG. 15A, while simulating the color perception tasks, in real-time (e.g., in step 1508), the computer device 140 also continuously tracks (e.g., in step 1510) (e.g., using the tracking module 1144), using the eye-tracking sensors, user responses (e.g., the responses 1148) and response times (e.g., the response times 1148) to the simulated tasks. Referring next to FIG. 15C, in some embodiments, the eye-tracking sensors may include (e.g., in step 1522) infrared cameras with high-frequency tracking of at least 120 Hz, millisecond latency, and/or sub-millimeter precision.

Referring back to FIG. 15A, the computer device 140 also evaluates (e.g., in step 1512) (e.g., using the evaluation/measurement module 1150) the tracked data for color perception performance (e.g., the color perception performance 1158). Referring to FIG. 15D, in some embodiments, the computer device 140 evaluates the tracked data by assessing (e.g., in step 1524) gaze direction, fixation points, and response times, measuring (e.g., in step 1526) color discrimination accuracy, calculating (e.g., in step 1528) reaction times across varying luminosities, and/or determining (e.g., in step 1530) error rates under specific conditions. In some embodiments, the computer device 140 assesses gaze direction by identifying frequent shifts in gaze that may indicate difficulty in maintaining focus on certain colors under specific conditions. In some embodiments, the computer device 140 assesses fixation points by identifying (e.g., in step 1532) longer fixation durations on particular colors or backgrounds that may suggest challenges in distinguishing these colors from their surroundings. In some embodiments, the computer device 140 evaluates the tracked data by assessing (e.g., in step 1534) color perception separately for different lighting conditions and background complexities. In some embodiments, the computer device 140 evaluates the tracked data by mapping (e.g., in step 1536) the user's gaze direction, fixation points, and response times to their color perception accuracy and adaptability.

Referring to FIG. 15E, in some embodiments, the computer device 140 also presents (e.g., in step 1538) a sequence of color perception tasks, wherein the tasks progress from low luminosity to high luminosity conditions. In some embodiments, the sequence of color perception tasks includes transitioning (e.g., in step 1540) between different backgrounds to assess adaptability in color perception. In some embodiments, the computer device 140 also generates (e.g., in step 1542) a color perception profile based on the evaluated tracked data, and/or provides (e.g., in step 1544) recommendations for improving color perception in challenging scenarios. In some embodiments, the recommendations include (e.g., in step 1546) suggestions for environmental modifications to enhance color perception. In some embodiments, the computer device 140 also compiles (e.g., in step 1548) a comprehensive report including detailed color perception capabilities, identified deficiencies, and recommendations for improving color perception. In some embodiments, the computer device 140 also calibrates (e.g., in step 1550) the system using a control group with known color perception profiles to establish baseline metrics.

Referring to FIG. 15F, in some embodiments, the computer device 140 also establishes (e.g., in step 1552) baseline performance metrics by comparing the user's color perception data with profiles of individuals with normal color vision, identifies (e.g., in step 1554) potential color perception deficiencies based on deviations from the established baseline, and/or provides (e.g., in step 1556) recommendations for further color vision evaluation if significant deviations are detected. In some embodiments, the computer device 140 also simulates (e.g., in step 1558) the effect of different environmental modifications in the virtual environment, allows (e.g., in step 1560) the user to experience and compare the simulated modifications in real-time, receives (e.g., in step 1562) user feedback on the simulated modifications, and/or provides (e.g., in step 1566) final recommendations that balance objective color perception data with subjective user preferences.

### VR Color Wavelength Sensitivity Testing System

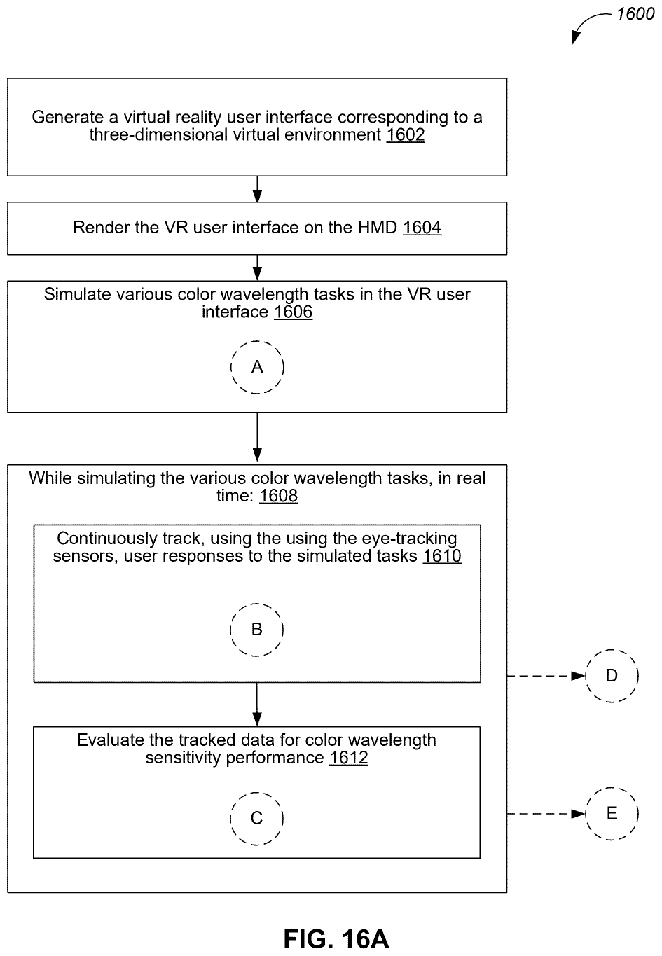

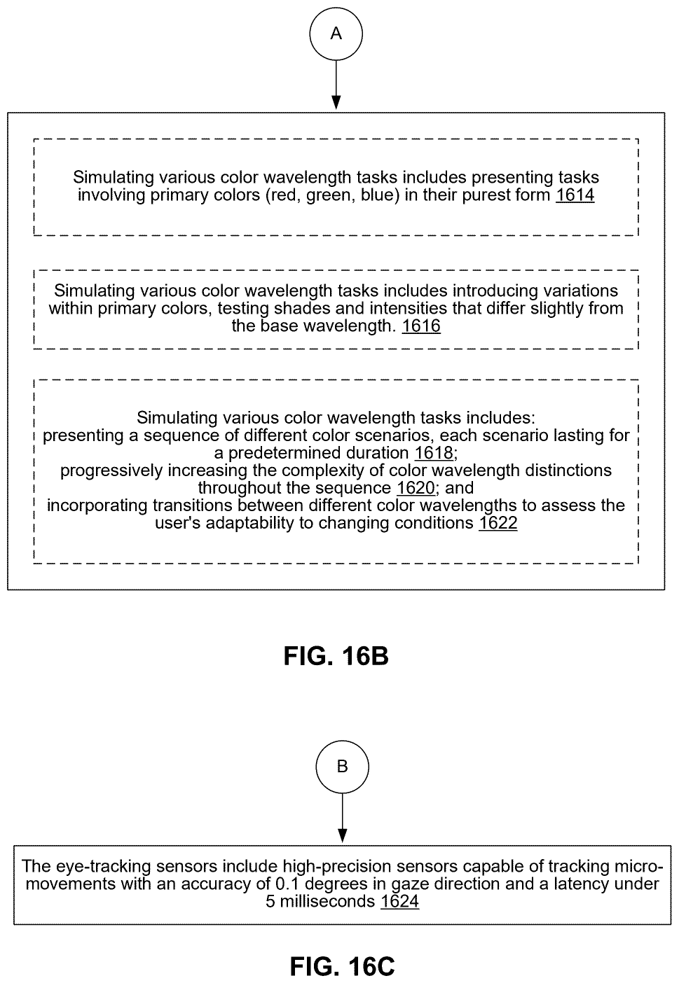

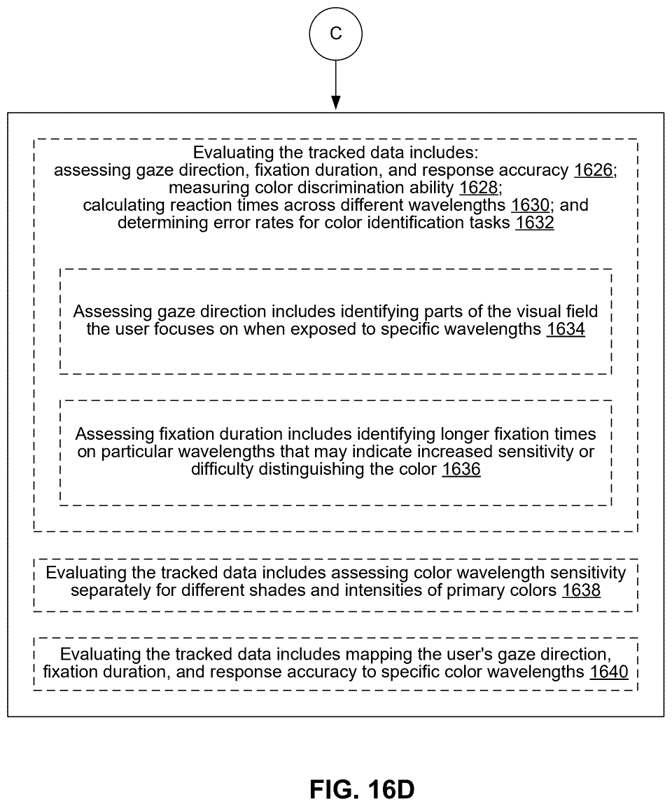

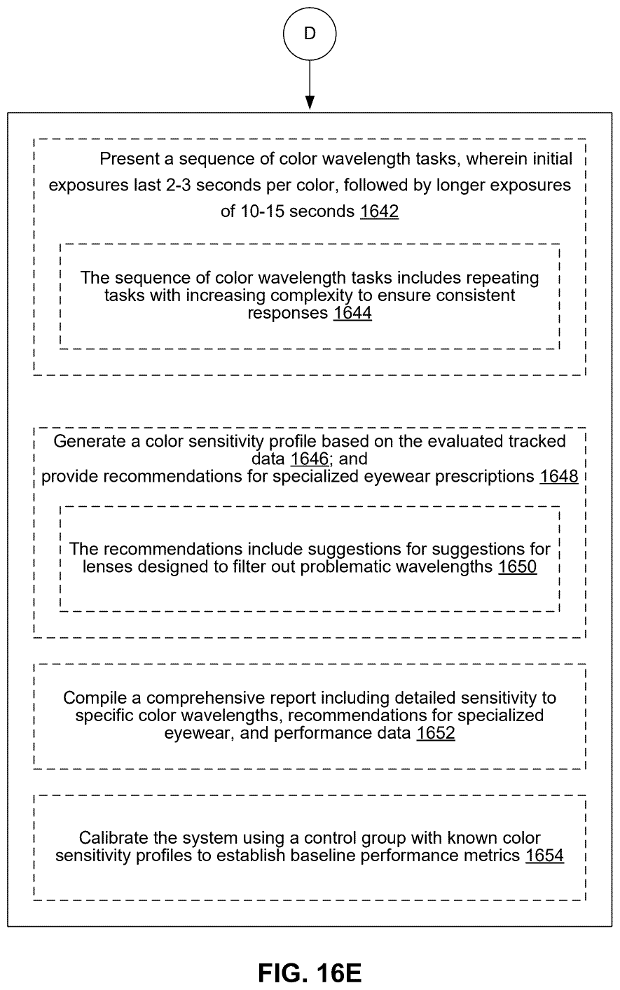

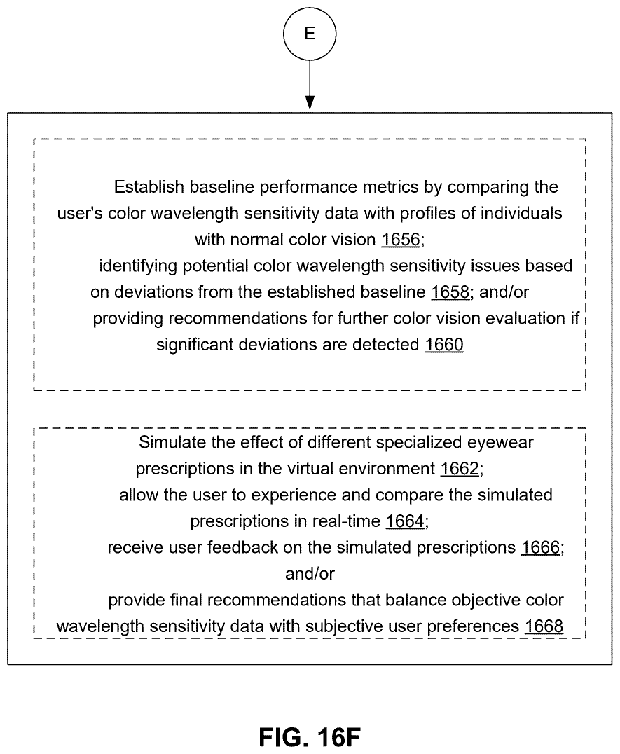

According to some embodiments, the vision test system 1100 described above is configured to test sensitivity to specific color wavelengths for specialized eyewear prescriptions. FIGS. 16A-16F show a flow diagram of an example process 1600 for testing sensitivity to specific color wavelengths for specialized eyewear prescriptions, according to some embodiments.

The computer device 140 (e.g., the computing device described above in reference to FIGS. 11A and 11B) generates (e.g., in step 1602) (e.g., using the UI module 1134) a VR user interface corresponding to a three-dimensional virtual environment (e.g., the environment 1136).

The computer device 140 also renders (e.g., in step 1604) (e.g., using the rendering module 1138) the VR user interface on the HMD 312A. Example details of the three-dimensional virtual environment and rendering the VR user interface are described above in reference to FIG. 12B, according to some embodiments.

The computer device 140 also simulates (e.g., in step 1606) (e.g., using the simulation module 1140), in the VR user interface, various color wavelength tasks (e.g., the scenarios 1142). Referring to FIG. 16B, in some embodiments, the computer device 140 simulates (e.g., in step 1614) various color wavelength tasks by presenting (e.g., in step 1614) tasks involving primary colors (red, green, blue) in their purest form. In some embodiments, the computer device 140 simulates various color wavelength tasks by introducing (e.g., in step 1616) variations within primary colors, testing shades and intensities that differ slightly from the base wavelength. in some embodiments, the computer device 140 simulates various color wavelength tasks by presenting (e.g., in step 1618) a sequence of different color scenarios, each scenario lasting for a predetermined duration, progressively increasing (e.g., in step 1620) the complexity of color wavelength distinctions throughout the sequence, and/or incorporating (e.g., in step 1622) transitions between different color wavelengths to assess the user's adaptability to changing conditions

Referring back to FIG. 16A, while simulating the color wavelength tasks, in real time (e.g., in step 1608), the computer device 140 also tracks (e.g., in step 1610) (e.g., using the tracking module 1144), using the eye-tracking sensors, user responses (e.g., the responses 1148) to the simulated tasks. Referring to FIG. 16C, in some embodiments, the eye-tracking sensors include (e.g., in step 1624) high-precision sensors capable of tracking micro-movements with an accuracy of 0.1 degrees in gaze direction and a latency under 5 ms.

Referring back to FIG. 16A, the computer device 140 also evaluates (e.g., in step 1610) (e.g., using the evaluation/measurement module 1150) the tracked data for color wavelength sensitivity performance (e.g., the color sensitivity performance 1156). Referring to FIG. 16D, in some embodiments, the computer device 140 evaluates the tracked data by assessing (e.g., in step 1626) gaze direction, fixation duration, and response accuracy, measuring (e.g., in step 1628) color discrimination ability, calculating (e.g., in step 1630) reaction times across different wavelengths, and/or determining (e.g., in step 1632) error rates for color identification tasks. In some embodiments, the computer device 140 assesses gaze direction by identifying (e.g., in step 1634) parts of the visual field the user focuses on when exposed to specific wavelengths. In some embodiments, the computer device 140 assesses fixation duration by identifying (e.g., in step 1636) longer fixation times on particular wavelengths that may indicate increased sensitivity or difficulty distinguishing the color. In some embodiments, the computer device 140 evaluates the tracked data by assessing (e.g., in step 1638) color wavelength sensitivity separately for different shades and intensities of primary colors. In some embodiments, the computer device 140 evaluates the tracked data by mapping (e.g., in step 1640) the user's gaze direction, fixation duration, and response accuracy to specific color wavelengths.

Referring to FIG. 16E, in some embodiments, the computer device 140 also presents (e.g., in step 1642) a sequence of color wavelength tasks. Initial exposures may last 2-3 seconds per color, followed by longer exposures of 10-15 seconds. In some embodiments, the sequence of color wavelength tasks includes repeating (e.g., in step 1644) tasks with increasing complexity to ensure consistent responses. In some embodiments, the computer device 140 also generates (e.g., in step 1646) a color sensitivity profile based on the evaluated tracked data, and/or provides (e.g., in step 1648) recommendations for specialized eyewear prescriptions. In some embodiments, the recommendations include (e.g., in step 1650) suggestions for lenses designed to filter out problematic wavelengths. In some embodiments, the computer device 140 also compiles (e.g., in step 1652) a comprehensive report including detailed sensitivity to specific color wavelengths, recommendations for specialized eyewear, and performance data. In some embodiments, the computer device 140 also calibrates (e.g., in step 1654) the system using a control group with established color sensitivity profiles to establish baseline performance metrics.

Referring next to FIG. 16F, in some embodiments, the computer device 140 also establishes (e.g., in step 1656) baseline performance metrics by comparing the user's color wavelength sensitivity data with profiles of individuals with normal color vision, identifies (e.g., in step 1658) potential color wavelength sensitivity issues based on deviations from the established baseline, and/or provides (e.g., in step 1660) recommendations for further color vision evaluation if significant deviations are detected. In some embodiments, the computer device 140 also simulates (e.g., in step 1662) the effect of different specialized eyewear prescriptions in the virtual environment, allows (e.g., in step 1664) the user to experience and compare the simulated prescriptions in real-time, receives (e.g., in step 1666) user feedback on the simulated prescriptions, and/or provides (e.g., in step 1668) final recommendations that balance objective color wavelength sensitivity data with subjective user preferences.

### VR Adaptive Eyewear Testing and Recommendation System

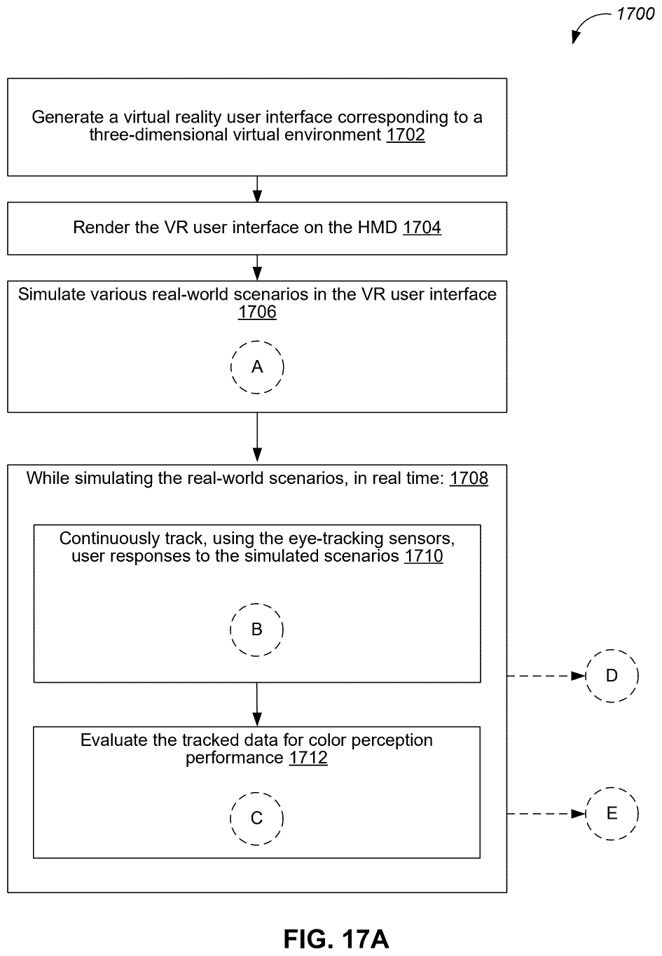

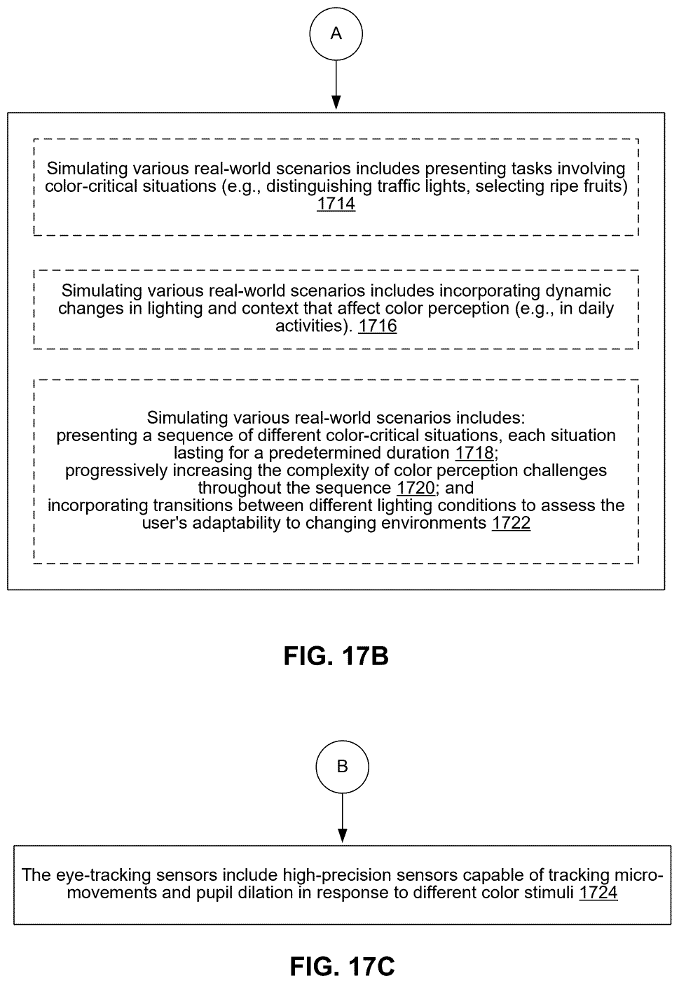

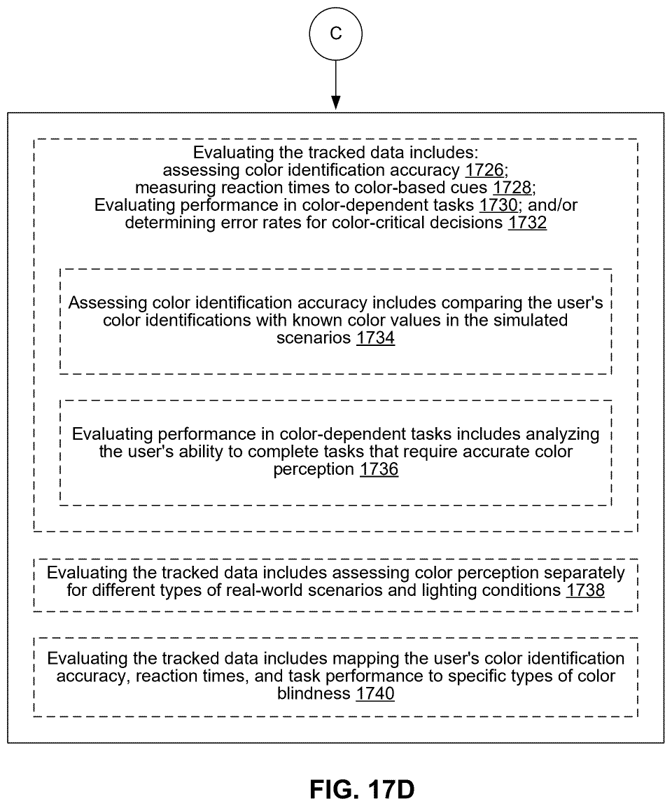

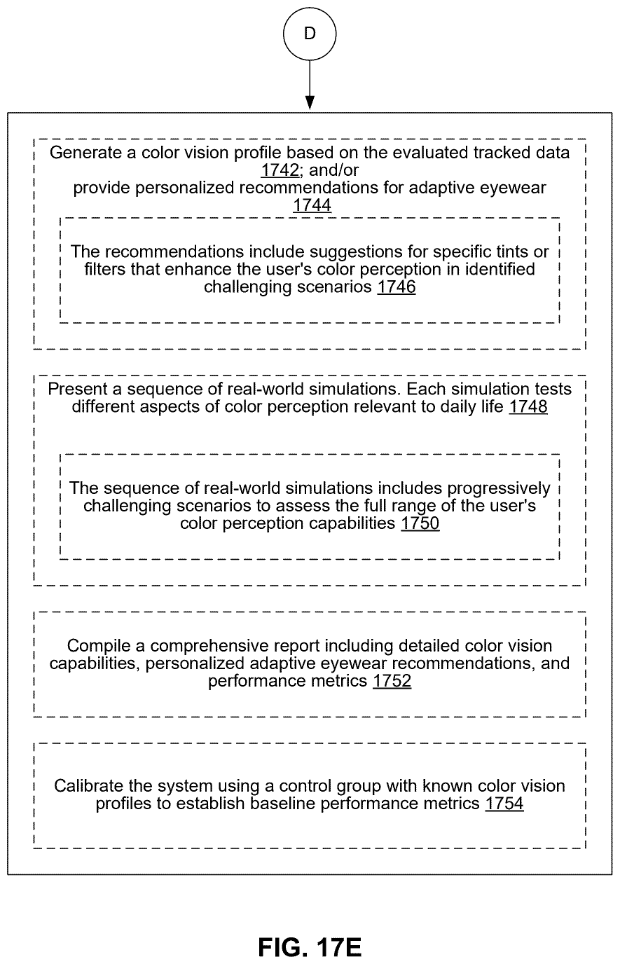

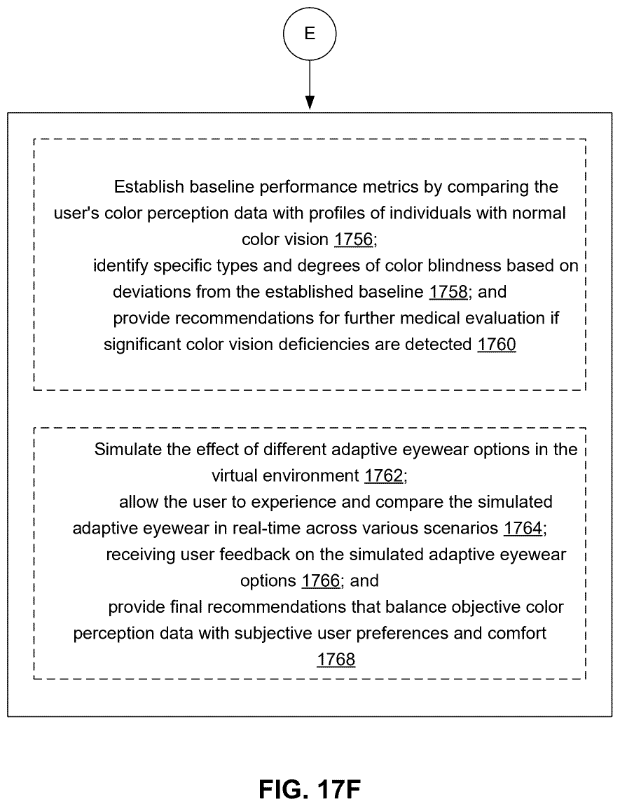

According to some embodiments, the vision test system 1100 described above is configured for testing and/or recommending adaptive eyewear for color blindness in real-world simulations. FIGS. 17A-17F show a flow diagram of an example process 1700 for testing and/or recommending adaptive eyewear for color blindness in real-world simulations, according to some embodiments.

The computer device 140 (e.g., the computing device described above in reference to FIGS. 11A and 11B) generates (e.g., in step 1702) (e.g., using the UI module 1134) a VR user interface corresponding to a three-dimensional virtual environment (e.g., the environment 1136).

Referring back to FIG. 17A, the computer device 140 also renders (e.g., in step 1704) (e.g., using the rendering module 1138) the VR user interface on the HMD 312A. Example details of the three-dimensional virtual environment and rendering the VR user interface are described above in reference to FIG. 12B, according to some embodiments.

The computer device 140 also simulates (e.g., in step 1706) (e.g., using the simulation module 1140), in the VR user interface, a plurality of real-world scenarios (e.g., the scenarios 1142). Referring to FIG. 17B, in some embodiments, the computer device 140 simulates various real-world scenarios by presenting (e.g., in step 1714) tasks involving color-critical situations (e.g., distinguishing traffic lights or selecting ripe fruits). In some embodiments, the computer device 140 simulates various real-world scenarios by incorporating (e.g., in step 1716) dynamic changes in lighting and context that affect color perception in daily activities. In some embodiments, the computer device 140 simulates various real-world scenarios by presenting (e.g., in step 1718) a sequence of different color-critical situations, each situation lasting for a predetermined duration, progressively increasing (e.g., in step 1720) the complexity of color perception challenges throughout the sequence, and/or incorporating (e.g., in step 1722) transitions between different lighting conditions to assess the user's adaptability to changing environments

Referring back to FIG. 17A, while simulating the real-world scenarios, in real-time (e.g., in step 1708), the computer device 140 also continuously tracks (e.g., in step 1710) (e.g., using the tracking module 1144), using the eye-tracking sensors, user responses (e.g., the responses 1148) to the simulated scenarios. Referring to FIG. 17C, in some embodiments, the eye-tracking sensors include (e.g., in step 1724) high-precision sensors capable of tracking micro-movements and pupil dilation in response to different color stimuli.

Referring back to FIG. 17A, the computer device 140 also evaluates (e.g., in step 1712) (e.g., using the evaluation/measurement module 1150) the tracked data for color perception performance (e.g., the color perception performance 1158). Referring next to FIG. 17D, in some embodiments, the computer device 140 evaluates the tracked data by assessing (e.g., in step 1726) color identification accuracy, measuring (e.g., in step 1728) reaction times to color-based cues, evaluating (e.g., in step 1730) performance in color-dependent tasks, and/or determining (e.g., in step 1732) error rates for color-critical decisions. In some embodiments, the computer device 140 assesses color identification accuracy by comparing (e.g., in step 1734) the user's color identifications with known color values in the simulated scenarios. In some embodiments, the computer device 140 evaluates performance in color-dependent tasks by analyzing (e.g., in step 1736) the user's ability to complete tasks that require accurate color perception. In some embodiments, the computer device 140 evaluates the tracked data by assessing (e.g., in step 1738) color perception separately for different types of real-world scenarios and lighting conditions. In some embodiments, the computer device 140 evaluates the tracked data by mapping (e.g., in step 1740) the user's color identification accuracy, reaction times, and task performance to specific types of color blindness.

Referring next to FIG. 17E, in some embodiments, the computer device 140 also generates (e.g., in step 1742) a color vision profile based on the evaluated tracked data, and/or provides (e.g., in step 1744) personalized recommendations for adaptive eyewear. In some embodiments, the recommendations include (e.g., in step 1746) suggestions for specific tints or filters that enhance the user's color perception in identified challenging scenarios. In some embodiments, the computer device 140 also presents (e.g., in step 1748) a sequence of real-world simulations. Each simulation may test different aspects of color perception relevant to daily life. In some embodiments, the sequence of real-world simulations includes progressively challenging (e.g., in step 1750) scenarios to assess the full range of the user's color perception capabilities. In some embodiments, the computer device 140 also compiles (e.g., in step 1752) a comprehensive report including detailed color vision capabilities, personalized adaptive eyewear recommendations, and performance metrics. In some embodiments, the computer device 140 also calibrates (e.g., in step 1754) the system using a control group with known color vision profiles to establish baseline performance metrics.

Referring next to FIG. 17F, in some embodiments, the computer device 140 also establishes (e.g., in step 1756) baseline performance metrics by comparing the user's color perception data with profiles of individuals with normal color vision, identifies (e.g., in step 1758) specific types and degrees of color blindness based on deviations from the established baseline, and/or provides (e.g., in step 1760) recommendations for further medical evaluation if significant color vision deficiencies are detected. In some embodiments, the computer device 140 also simulates (e.g., in step 1762) the effect of different adaptive eyewear options in the virtual environment, allows (e.g., in step 1764) the user to experience and compare the simulated adaptive eyewear in real-time across various scenarios, receives (e.g., in step 1766) user feedback on the simulated adaptive eyewear options, and/or provides (e.g., in step 1768) final recommendations that balance objective color perception data with subjective user preferences and comfort.

### Example Vision Test Process

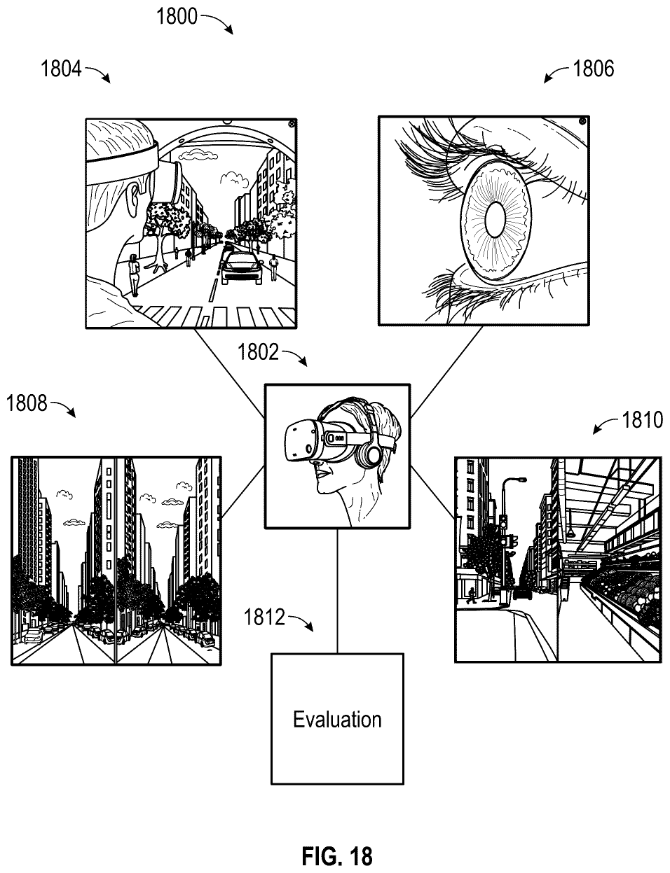

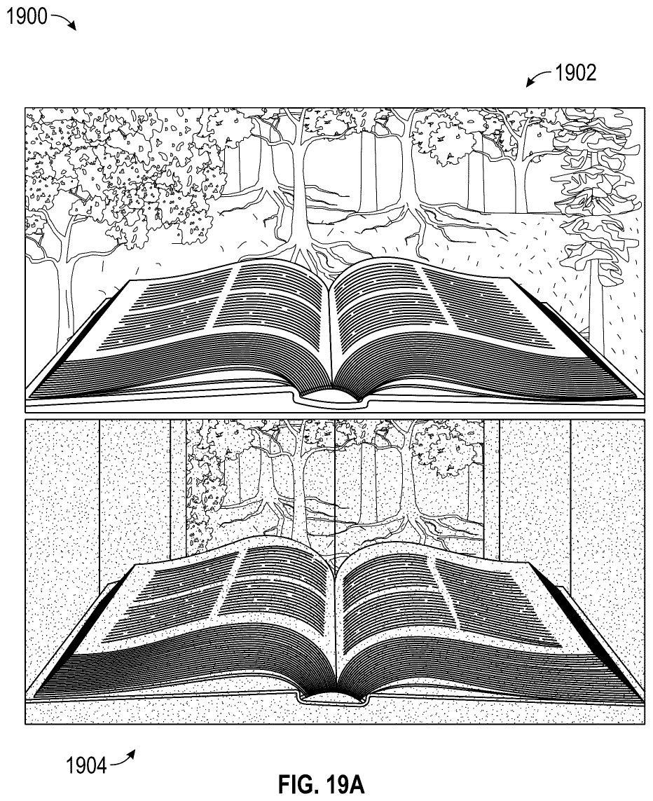

FIG. 18 is a schematic diagram showing an example vision test 1800, in accordance with some embodiments. The illustration 1802 shows a person wearing a VR headset (HMD). The VR headset may include eye-tracking cameras. As shown in the illustration 1804, the user's view through the HMD may show a three-dimensional virtual environment. An example of an environment is shown in the illustration 1810. The illustration 1806 shows a close-up of an eye that may be tracked by the eye-tracking cameras, which may track eye movements, such as saccades, fixations, and smooth pursuit. The illustration 1808 shows example scenarios that may be displayed in the HMD for evaluation response. Based on responses, the system may perform various evaluations (e.g., in step 1812).

### Example VR Light Sensitivity Testing and LCD Tinted Lens Prescription System

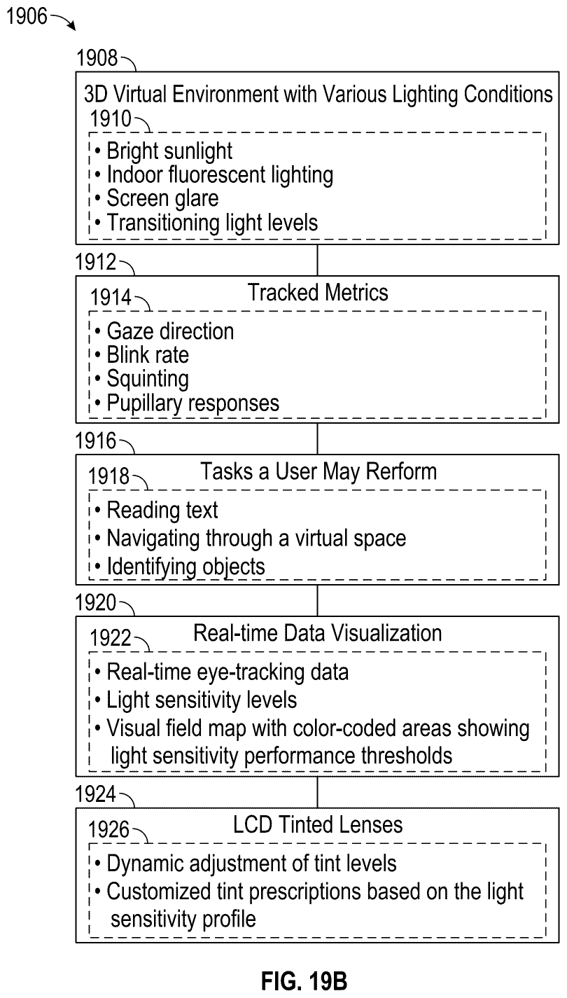

FIG. 19A shows illustrations of example visual scenarios 1900, for VR light sensitivity testing and LCD tinted lens prescription system, according to some embodiments. The illustration shows two panels showing an open textbook with written text. One panel 1902 shows the textbook under bright sunlight in an outdoor setting. The other panel 1904 shows the textbook under indoor dim lighting under a study lamp.

FIG. 19B is a block diagram of example components 1906 for a VR light sensitivity testing and LCD tinted lens prescription system, according to some embodiments. Some embodiments can include 3D virtual environment with various lighting conditions 1908, which may include, for example, bright sunlight, indoor fluorescent lighting, screen glare, and/or transitioning light levels. Some embodiments can include tracked metrics 1912, which may include, for example, gaze direction, blink rate, squinting, and/or pupillary responses 1914. In some embodiments, tasks 1916 a user may perform, may include, for example, reading text, navigating through a virtual space, and/or identifying objects 1918. In some embodiments, real-time data visualization 1920, which may include, for example, real-time eye-tracking data, light sensitivity levels, and/or visual field map with color-coded areas showing light sensitivity performance thresholds 1922. Some embodiments can include LCD tinted lenses 1924, which may include, for example, dynamic adjustment of tint levels, and/or customized tint prescriptions based on the light sensitivity profile 1926.

### Example VR Lens Tint Recommendation Through Interactive Vision Sensitivity Test

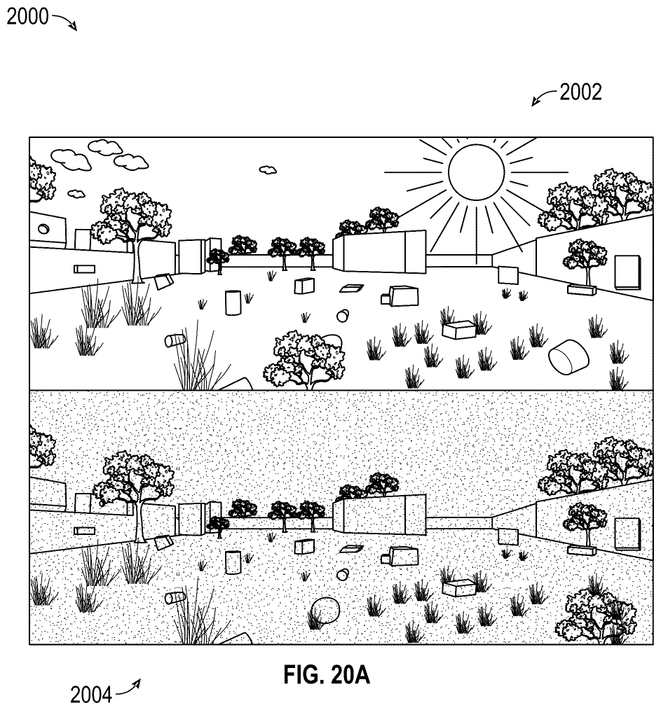

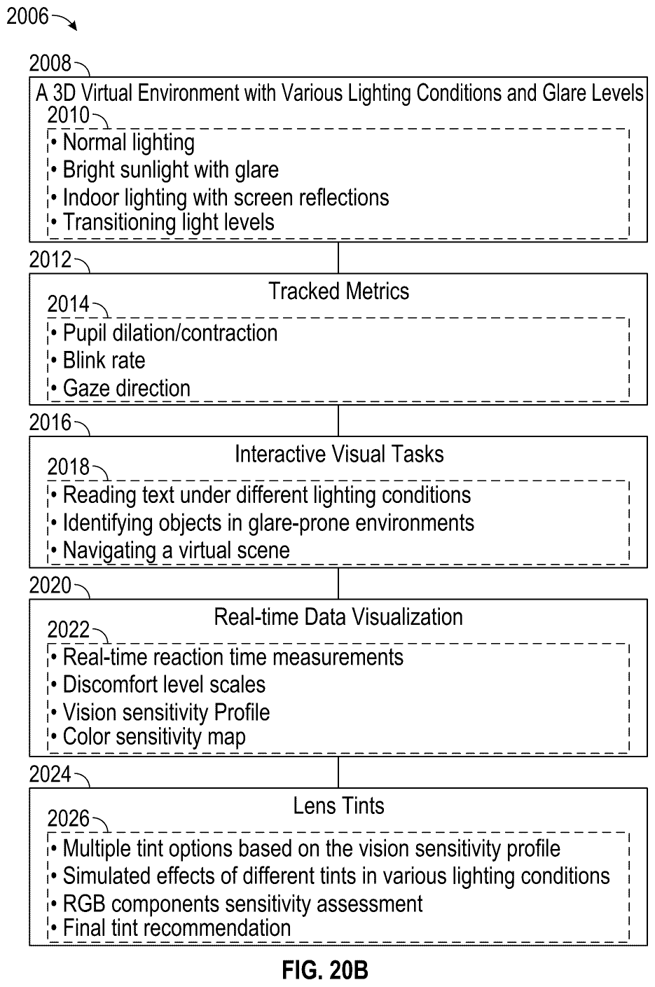

FIG. 20A shows illustrations of example photorealistic view glare-prone environments 2000 for VR lens tint recommendation through interactive vision sensitivity test, according to some embodiments. The environments show two panels, each showing some objects. One panel 2002 shows the environment in bright sunlight with glare, the other panel 2004 shows the environment in normal light.

FIG. 20B is a block diagram of example components 2006 for VR lens tint recommendation through interactive vision sensitivity test, according to some embodiments. Some embodiments can include 3D virtual environment with various lighting conditions and glare levels 2008, which may include, for example, normal lighting, bright sunlight with glare, indoor lighting with screen reflections, and/or transitioning light levels 2010. Some embodiments can include tracked metrics 2012, which may include, for example, gaze direction, blink rate, and/or pupillary dilation and/or contraction 2014. Some embodiments can include interactive visual tasks 2016, which may include, for example, reading text under different lighting conditions, identifying objects in glare-prone environments, and/or navigating a virtual scene 2018. Some embodiments can include real-time data visualization 2020, which may include, for example, real-time reaction time measurements, discomfort level scales, vision sensitivity profile, and/or color sensitivity map 2022. Some embodiments can include lens tints 2024, which may include, for example, multiple tint options based on the vision sensitivity profile, simulated effects of different tints in various lighting conditions, RGB component sensitivity assessment, and/or final tint recommendation.

### Example VR-Enabled Color Blindness Test Using Color-Coded Challenges and Puzzles

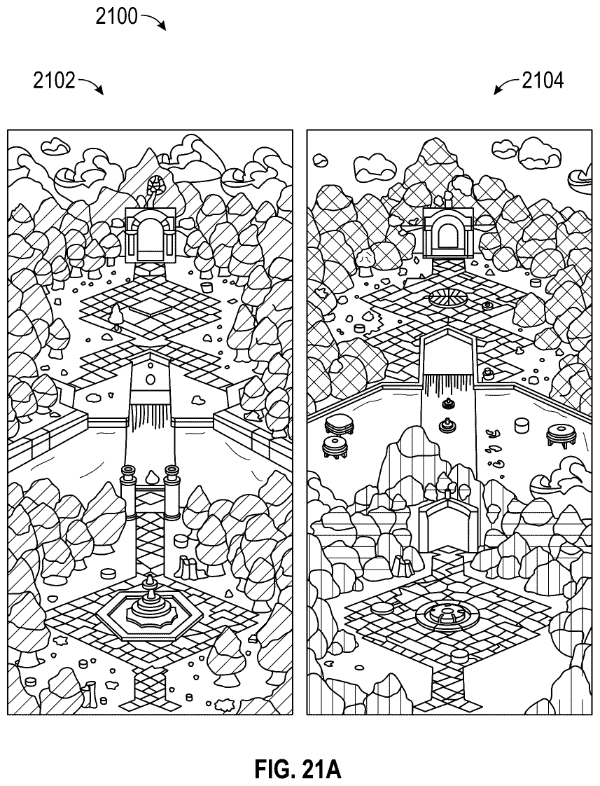

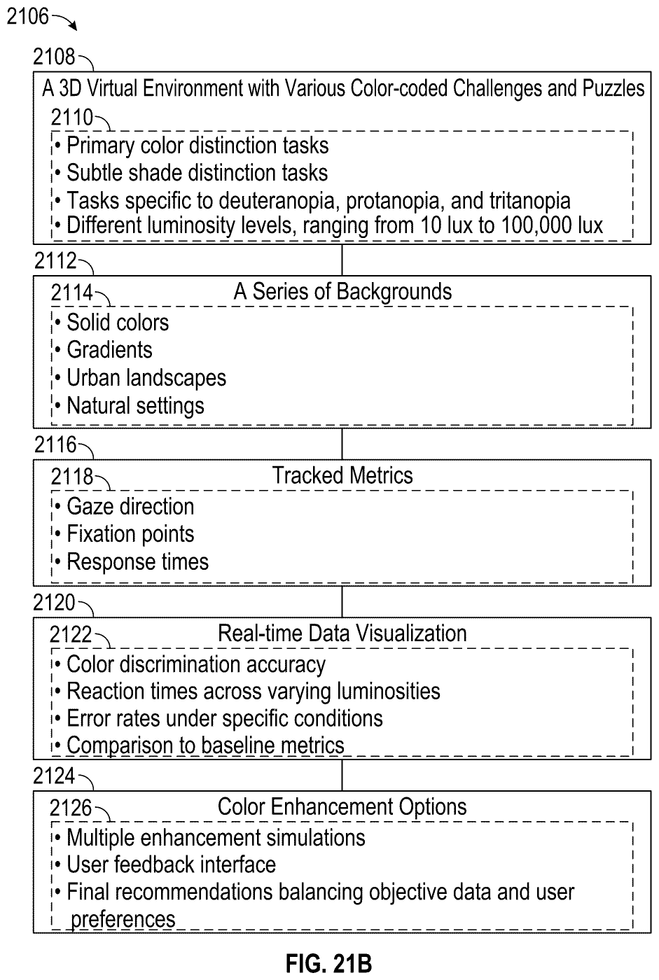

FIG. 21A shows illustrations of example 3D virtual environments 2100 for VR-enabled color blindness test using color-coded challenges and puzzles, according to some embodiments. The figure shows two panels, each displaying different 3D virtual environments with various color-coded challenges and puzzles. One panel 2102 shows one set of challenges and puzzles, while the other panel 2104 shows a different set.

FIG. 21B is a block diagram of example components 2106 for VR-enabled color blindness test using color-coded challenges and puzzles, according to some embodiments. Some embodiments can include a 3D virtual environment with various color-coded challenges and puzzles 2108, which may include, for example, primary color distinction tasks, subtle shade distinction tasks, tasks specific to deuteranopia, protanopia, and tritanopia, and/or different luminosity levels, ranging from 10 lux to 100,000 lux, 2110. Some embodiments can include a series of backgrounds 2112, which may include, for example, solid colors, gradients, urban landscapes, and/or natural settings 2114. Some embodiments can include tracked metrics 2116, which may include, for example, gaze direction, fixation points, and/or response times 2118. Some embodiments can include real-time data visualization 2120, which may include, for example, color discrimination accuracy, reaction times across varying luminosities, error rates under specific conditions, and/or comparison to baseline metrics 2122. Some embodiments can include color enhancement options 2124, which may include, for example, multiple enhancement simulations, user feedback interface, and/or final recommendations balancing objective data and user preferences 2126.

### Example VR-Based Color Perception Evaluation System

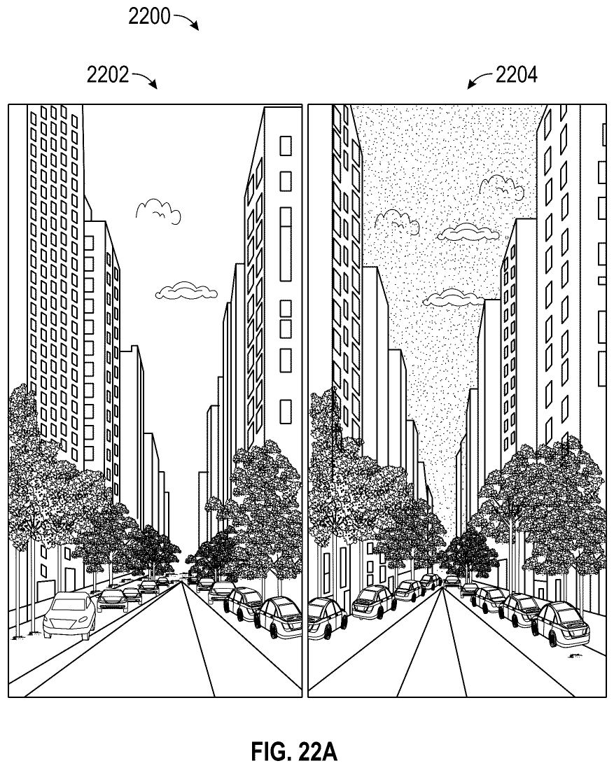

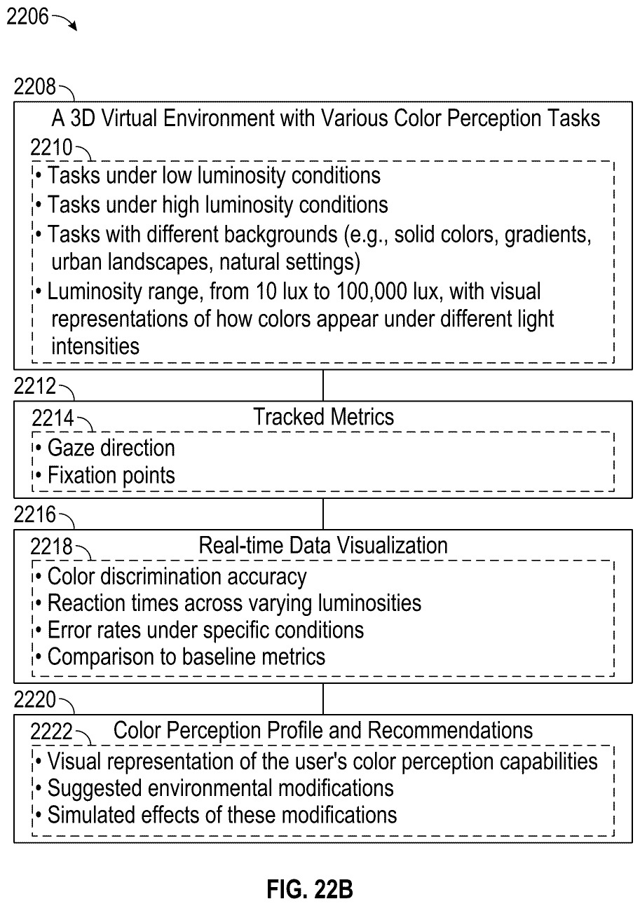

FIG. 22A shows illustrations of example backgrounds 2200 for VR-based color perception evaluation system, according to some embodiments. The figure shows two panels, each displaying different backgrounds under various luminosity conditions. One panel 2202 shows one set of backgrounds (e.g., solid colors, gradients) under certain luminosity conditions, while the other panel 2204 shows a different set of backgrounds (e.g., urban landscapes, natural settings) under different luminosity conditions.

FIG. 22B is a block diagram of example components 2206 for VR-based color perception evaluation system, according to some embodiments. Some embodiments can include a 3D virtual environment with various color perception tasks 2208, which may include, for example, tasks under low luminosity conditions, tasks under high luminosity conditions, tasks with different backgrounds (e.g., solid colors, gradients, urban landscapes, natural settings), and/or luminosity range, from 10 lux to 100,000 lux, with visual representations of how colors appear under different light intensities 2210. Some embodiments can include tracked metrics 2212, which may include, for example, gaze direction and/or fixation points 2214. Some embodiments can include real-time data visualization 2216, which may include, for example, color discrimination accuracy, reaction times across varying luminosities, error rates under specific conditions, and/or comparison to baseline metrics 2218. Some embodiments can include color perception profile and recommendations 2220, which may include, for example, visual representation of the user's color perception capabilities, suggested environmental modifications, and/or simulated effects of these modifications 2222.

### Example VR-Based Color Wavelength Sensitivity Evaluation System

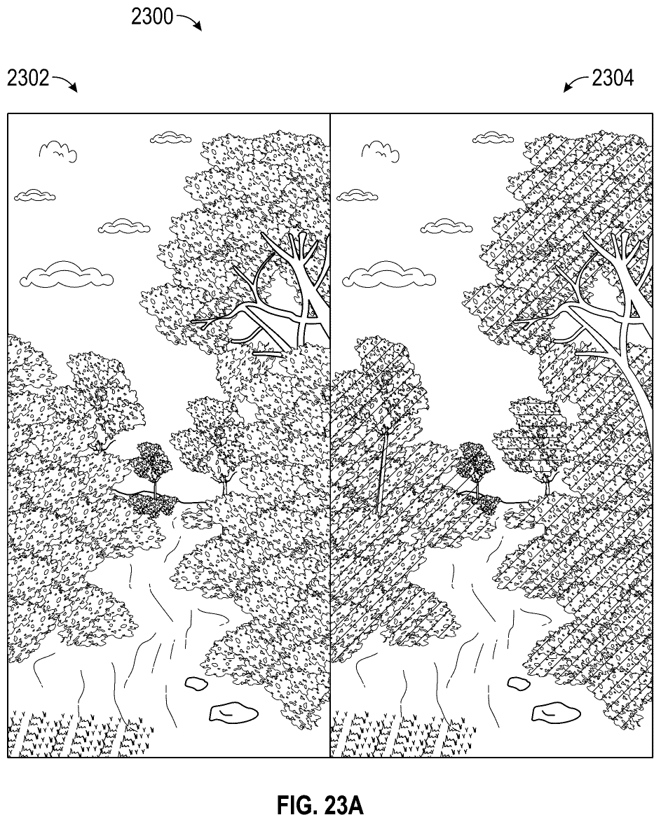

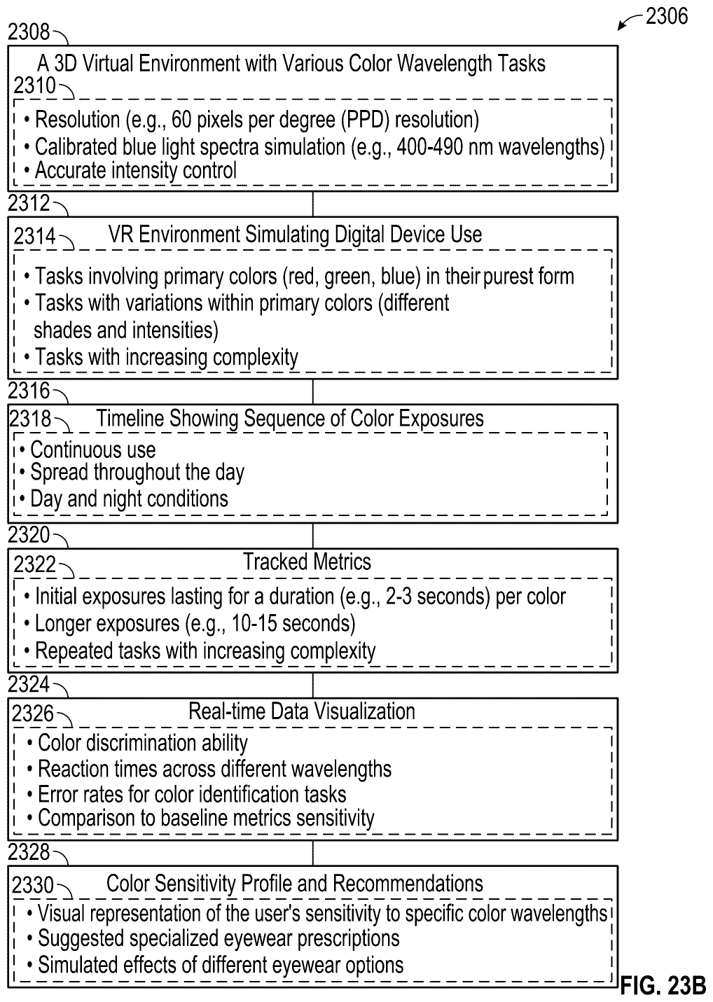

FIG. 23A shows illustrations of example color presentations 2300 for VR-based color wavelength sensitivity evaluation system, according to some embodiments. The figure shows two panels. One panel 2302 displays only primary colors (red, green, blue) in their purest form, while the other panel 2304 shows variations of the primary colors with different shades and intensities.

FIG. 23B is a block diagram of example components 2306 for VR-based color wavelength sensitivity evaluation system, according to some embodiments. Some embodiments can include a 3D virtual environment with various color wavelength tasks 2308, which may include, for example, resolution (e.g., 60 pixels per degree (PPD) resolution), calibrated blue light spectra simulation (e.g., 400-490 nm wavelengths), and/or accurate intensity control 2310. Some embodiments can include a VR environment simulating digital device use 2312, which may include, for example, tasks involving primary colors (red, green, blue) in their purest form, tasks with variations within primary colors (different shades and intensities), and/or tasks with increasing complexity 2314. Some embodiments can include a timeline showing sequence of color exposures 2316, which may include, for example, continuous use, spread throughout the day, and/or day and night conditions 2318. Some embodiments can include tracked metrics 2320, which may include, for example, initial exposures lasting for a duration (e.g., 2-3 seconds) per color, longer exposures (e.g., 10-15 seconds), and/or repeated tasks with increasing complexity 2322. Some embodiments can include real-time data visualization 2324, which may include, for example, color discrimination ability, reaction times across different wavelengths, error rates for color identification tasks, and/or comparison to baseline metrics sensitivity 2326. Some embodiments can include color sensitivity profile and recommendations 2328, which may include, for example, visual representation of the user's sensitivity to specific color wavelengths, suggested specialized eyewear prescriptions, and/or simulated effects of different eyewear options 2330.

### Example VR-Based Adaptive Eyewear Recommendation System for Color Blindness

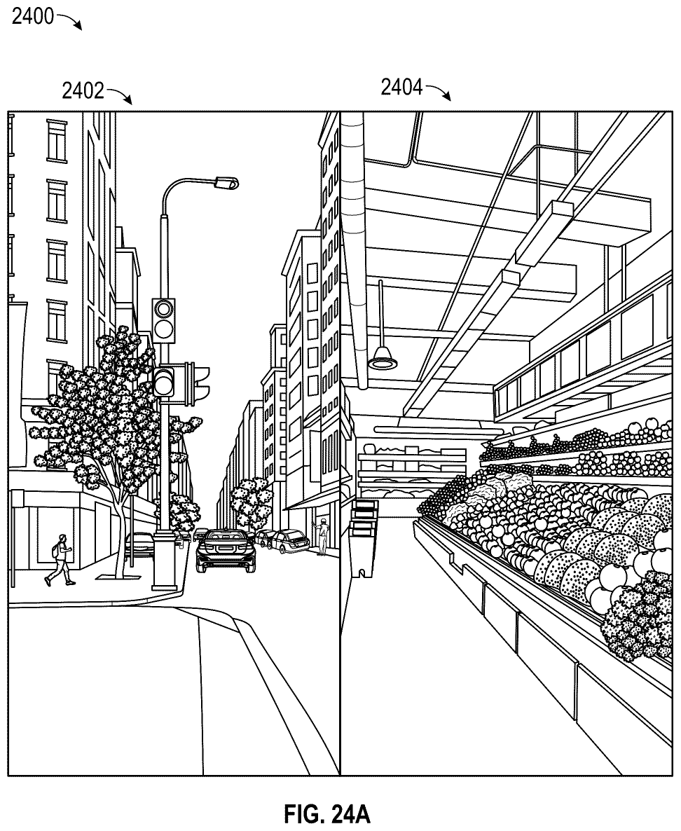

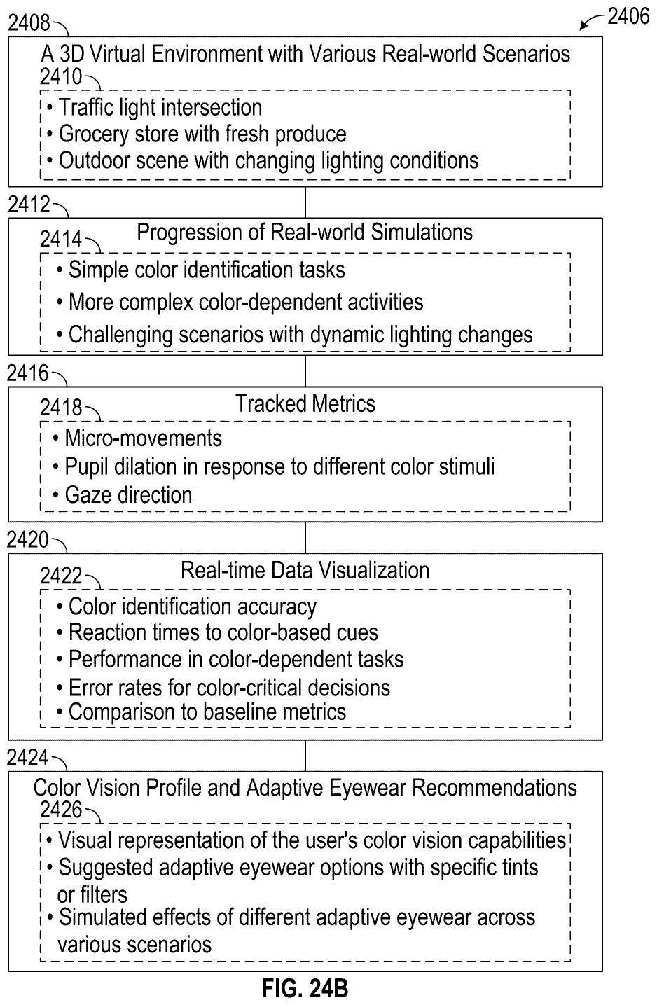

FIG. 24A shows illustrations of example real-world scenarios 2400 for VR-based adaptive eyewear recommendation system for color blindness, according to some embodiments. The figure shows two panels. One panel 2402 displays a traffic light intersection with color signals, while the other panel 2404 shows a grocery store with fresh produce of different colors.

FIG. 24B is a block diagram of example components 2406 for VR-based adaptive eyewear recommendation system for color blindness, according to some embodiments. Some embodiments can include a 3D virtual environment with various real-world scenarios 2408, which may include, for example, a traffic light intersection, a grocery store with fresh produce, and/or an outdoor scene with changing lighting conditions 2410. Some embodiments can include a progression of real-world simulations 2412, which may include, for example, simple color identification tasks, more complex color-dependent activities, and/or challenging scenarios with dynamic lighting changes 2414. Some embodiments can include tracked metrics 2416, which may include, for example, micro-movements, pupil dilation in response to different color stimuli, and/or gaze direction 2418. Some embodiments can include real-time data visualization 2420, which may include, for example, color identification accuracy, reaction times to color-based cues, performance in color-dependent tasks, error rates for color-critical decisions, and/or comparison to baseline metrics 2422. Some embodiments can include color vision profile and adaptive eyewear recommendations 2424, which may include, for example, visual representation of the user's color vision capabilities, suggested adaptive eyewear options with specific tints or filters, and/or simulated effects of different adaptive eyewear across various scenarios 2426.

### Illustration of Subject Technology As Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.
Clause 1. A method of implementing a virtual reality (VR) system for testing light sensitivity and prescribing customized LCD tinted lenses, comprising: at an electronic device including a head-mounted display and eye-tracking sensors: generating a VR user interface corresponding to a three-dimensional virtual environment; rendering the VR user interface on the head-mounted display; simulating various lighting conditions sequentially in the VR user interface; and while simulating the various lighting conditions, in real time: continuously tracking, using the eye-tracking sensors, gaze direction, blink rate, squinting, and pupillary responses to the simulated lighting conditions; and evaluating the tracked data for light sensitivity performance.
Clause 2. The method of Clause 1, wherein simulating various lighting conditions comprises simulating one or more conditions selected from the group consisting of: bright sunlight, indoor fluorescent lighting, screen glare, transitioning light levels, and mixed light sources.
Clause 3. The method of Clauses 1 or 2, wherein simulating various lighting conditions comprises varying light intensities ranging from 50 lux to 100,000 lux.
Clause 4. The method of any of Clauses 1-3, wherein simulating various lighting conditions comprises simulating different types of light sources including fluorescent, LED, and natural sunlight.
Clause 5. The method of any of Clauses 1-4, further comprising presenting one or more tasks in the virtual environment, wherein the tasks are selected from the group consisting of: reading tasks, navigating virtual environments, and object identification.
Clause 6. The method of any of Clauses 1-5, wherein the eye-tracking sensors track eye movements with sub-millimeter precision, have a latency of less than 5 ms, and operate at a tracking frequency of 120 Hz or higher.
Clause 7. The method of any of Clauses 1-6, wherein evaluating the tracked data comprises: mapping eye-tracking data to light sensitivity levels; assessing gaze direction, blink rate, squinting, and pupillary response in relation to different lighting conditions; and quantifying vision drops across different visual fields.
Clause 8. The method of any of Clauses 1-7, further comprising: processing the tracked data using algorithms for measuring reaction time, assessing discomfort, and evaluating visual performance under different lighting conditions.
Clause 9. The method of any of Clauses 1-8, further comprising: generating a light sensitivity profile based on the evaluated tracked data; and customizing LCD tinted lens prescriptions based on the light sensitivity profile.
Clause 10. The method of any of Clauses 1-9, wherein customizing LCD tinted lens prescriptions comprises dynamically adjusting lens tint levels in real-time during testing to determine optimal tint levels for different lighting conditions.
Clause 11. The method of any of Clauses 1-10, further comprising using LCD tinted lenses to dynamically adjust tint levels based on the evaluated tracked data.
Clause 12. The method of any of Clauses 1-11, further comprising compiling a comprehensive report including recommended lens settings, detailed light sensitivity insights, and performance metrics under various lighting conditions.
Clause 13. The method of any of Clauses 1-12, wherein evaluating the tracked data includes assessing light sensitivity separately for each eye and in different quadrants of the visual field.
Clause 14. The method of any of Clauses 1-13, wherein tracking using the eye-tracking sensors comprises tracking eye movements using infrared cameras capable of tracking the eye movements with sub-millimeter precision, the infrared cameras having a latency of less than 5 ms and operating at a tracking frequency of 120 Hz or higher.
Clause 15. The method of any of Clauses 1-14, further comprising using artificial intelligence algorithms to dynamically adjust the simulated lighting conditions based on real-time analysis of the user's light sensitivity performance.
Clause 16. The method of any of Clauses 1-15, wherein evaluating light sensitivity performance includes generating a visual field map that color-codes areas showing light sensitivity performance across different lighting conditions.
Clause 17. The method of any of Clauses 1-16, wherein simulating various lighting conditions comprises: presenting a sequence of different lighting scenarios, each scenario lasting between a few seconds to several minutes; progressively increasing the complexity and intensity of the lighting conditions throughout the sequence; and incorporating transitions between different lighting conditions to assess the user's adaptability to changing light levels.
Clause 18. The method of any of Clauses 1-17, further comprising: establishing baseline performance metrics by comparing the user's light sensitivity data with profiles of individuals with normal light sensitivity and those with known light sensitivity conditions; identifying potential light sensitivity issues or conditions based on deviations from the established baseline; and providing recommendations for further medical evaluation if significant deviations are detected.
Clause 19. A method of implementing a virtual reality (VR) system for recommending lens tints through an interactive vision sensitivity test, comprising: at an electronic device including a head-mounted display (HMD) and a camera: generating a VR user interface corresponding to a three-dimensional virtual environment; rendering the VR user interface on the head-mounted display; simulating various lighting conditions and glare levels sequentially in the VR user interface; and while simulating the various lighting conditions and glare levels, in real time: continuously tracking, using the eye-tracking sensors, user responses to the simulated lighting conditions and glare levels; and evaluating the tracked data for vision sensitivity performance.
Clause 20. The method of Clause 19, wherein simulating various lighting conditions comprises varying light intensities ranging from 50 lux to 100,000 lux.
Clause 21. The method of Clauses 19 or 20, wherein simulating various glare levels comprises simulating conditions ranging from mild indirect light reflections to severe direct sunlight glare.
Clause 22. The method of any of Clauses 19-21, further comprising presenting one or more interactive visual tasks in the virtual environment, wherein the tasks are selected from the group consisting of: reading under different lighting conditions, identifying objects in glare-prone environments, and navigating virtual scenes.
Clause 23. The method of Clause 22, wherein the interactive visual tasks are sequenced from less to more challenging, gradually increasing light intensity and glare.
Clause 24. The method of any of Clauses 19-23, wherein evaluating the tracked data comprises: measuring reaction time to changes in lighting conditions; assessing discomfort levels through user feedback; and evaluating visual performance under different lighting conditions.
Clause 25. The method of Clause 24, wherein measuring reaction time comprises targeting reaction times of under one second.
Clause 26. The method of Clause 24, wherein assessing discomfort levels comprises converting user feedback into numerical scales.
Clause 27. The method of any of Clauses 19-26, further comprising: generating a vision sensitivity profile based on the evaluated tracked data; and recommending lens tints based on the vision sensitivity profile.
Clause 28. The method of Clause 27, wherein recommending lens tints comprises: assessing user sensitivity to specific RGB (Red, Green, Blue) components; and applying conversion factors that map sensitivity data to specific tint percentages.
Clause 29. The method of Clause 27, further comprising assigning confidence levels to each recommended tint percentage.
Clause 30. The method of any of Clauses 19-29, further comprising compiling a comprehensive report including detailed lens tint recommendations and a light sensitivity profile.
Clause 31. The method of any of Clauses 19-30, wherein evaluating the tracked data includes assessing vision sensitivity separately for different visual tasks and environments.
Clause 32. The method of any of Clauses 19-31, further comprising using artificial intelligence algorithms to dynamically adjust the simulated lighting conditions and glare levels based on real-time analysis of the user's vision sensitivity performance.
Clause 33. The method of any of Clauses 19-32, wherein simulating various lighting conditions and glare levels comprises: presenting a sequence of different lighting and glare scenarios, each scenario lasting for a predetermined duration; progressively increasing the complexity and intensity of the lighting conditions and glare levels throughout the sequence; and incorporating transitions between different scenarios to assess the user's adaptability to changing light and glare conditions.
Clause 34. The method of any of Clauses 19-33, further comprising: establishing baseline performance metrics by comparing the user's vision sensitivity data with profiles of individuals with normal vision sensitivity; identifying potential vision sensitivity issues based on deviations from the established baseline; and providing recommendations for further vision evaluation if significant deviations are detected.
Clause 35. The method of any of Clauses 19-34, further comprising: generating a color sensitivity map based on the user's responses to different color components under various lighting conditions and glare levels, wherein the color sensitivity map represents the user's sensitivity to specific wavelengths of light, wherein the color sensitivity map is used to fine-tune the lens tint recommendations; and wherein the color sensitivity map is presented as part of the comprehensive report, providing a visual representation of the user's color-specific light sensitivities.
Clause 36. The method of any of Clauses 19-35, wherein recommending lens tints comprises: generating multiple tint options based on the vision sensitivity profile; simulating the effect of each tint option in the virtual environment under various lighting conditions and glare levels; allowing the user to experience and compare the simulated tint options in real-time; receiving user feedback on the simulated tint options; refining the tint recommendations based on the user feedback; and providing a final tint recommendation that balances objective vision sensitivity data with subjective user preferences.
Clause 37. A method of implementing a virtual reality (VR) system for evaluating color perception, comprising: at an electronic device including a head-mounted display (HMD) and eye-tracking sensors: generating a VR user interface corresponding to a three-dimensional virtual environment; rendering the VR user interface on the head-mounted display; simulating various color-coded challenges and puzzles under varying luminosities and backgrounds in the VR user interface; and while simulating the color-coded challenges and puzzles, in real time: continuously tracking, using the eye-tracking sensors, user responses to the simulated challenges and puzzles; and evaluating the tracked data for color perception performance.
Clause 38. The method of Clause 37, wherein simulating various color-coded challenges and puzzles comprises presenting tasks specific to different types of color blindness, including deuteranopia, protanopia, and tritanopia.
Clause 39. The method of any of Clauses 37 or 38, wherein simulating various luminosities comprises varying light intensities ranging from 10 lux to 100,000 lux.
Clause 40. The method of any of Clauses 37-39, wherein simulating various backgrounds comprises presenting solid colors, gradients, and real-world scenes including urban landscapes and natural settings.
Clause 41. The method of any of Clauses 37-40, further comprising presenting a sequence of color differentiation tasks, wherein the tasks are ordered from easier primary color distinctions to more challenging subtle shade distinctions.
Clause 42. The method of any of Clauses 37-41, wherein the sequence of color differentiation tasks includes increasing complexity by randomizing colors and patterns to ensure adaptability and true deficiency identification.
Clause 43. The method of any of Clauses 37-42, wherein evaluating the tracked data comprises: assessing gaze direction, fixation points, and response times; measuring color discrimination accuracy; calculating reaction times across varying luminosities; and determining error rates under specific conditions.
Clause 44. The method of Clause 43, wherein assessing gaze direction comprises identifying frequent shifts in gaze that may indicate difficulty in maintaining focus on certain colors under specific conditions.
Clause 45. The method of Clause 43, wherein assessing fixation points comprises identifying longer fixation durations on particular colors or backgrounds that may suggest challenges in distinguishing these colors from their surroundings.
Clause 46. The method of any of Clauses 37-45, further comprising: generating a color vision profile based on the evaluated tracked data; and providing recommendations for corrective measures or adaptive strategies.
Clause 47. The method of Clause 46, wherein the recommendations include suggestions for environmental modifications to enhance color perception in challenging scenarios.
Clause 48. The method of any of Clauses 37-47, further comprising compiling a comprehensive report including a detailed color vision profile, identified deficiencies, and recommendations for improving color perception.
Clause 49. The method of any of Clauses 37-48, wherein evaluating the tracked data includes assessing color perception separately for different lighting conditions and background complexities.
Clause 50. The method of any of Clauses 37-49, further comprising calibrating the system using a control group with known color perception profiles to establish baseline metrics.
Clause 51. The method of any of Clauses 37-50, wherein simulating various color-coded challenges and puzzles comprises: presenting a sequence of different scenarios, each scenario lasting for a predetermined duration; progressively increasing the complexity of color distinctions throughout the sequence; and incorporating transitions between different luminosities and backgrounds to assess the user's adaptability to changing conditions.
Clause 52. The method of any of Clauses 37-51, further comprising: establishing baseline performance metrics by comparing the user's color perception data with profiles of individuals with normal color vision; identifying potential color vision deficiencies based on deviations from the established baseline; and providing recommendations for further color vision evaluation if significant deviations are detected.
Clause 53. The method of any of Clauses 37-52, wherein the eye-tracking sensors comprise infrared cameras with high-frequency tracking of at least 120 Hz, millisecond latency, and sub-millimeter precision.
Clause 54. The method of any of Clauses 37-53, further comprising: generating multiple color enhancement options based on the color vision profile; simulating the effect of each enhancement option in the virtual environment under various luminosities and backgrounds; allowing the user to experience and compare the simulated enhancement options in real-time; receiving user feedback on the simulated enhancement options; and providing final recommendations that balance objective color perception data with subjective user preferences.
Clause 55. A method of implementing a virtual reality (VR) system for evaluating color perception, comprising: at an electronic device including a head-mounted display (HMD) and eye-tracking sensors: generating a VR user interface corresponding to a three-dimensional virtual environment; rendering the VR user interface on the head-mounted display; simulating various color perception tasks under varying luminosities and backgrounds in the VR user interface; and while simulating the color perception tasks, in real time: continuously tracking, using the eye-tracking sensors, user responses to the simulated tasks; and evaluating the tracked data for color perception performance.
Clause 56. The method of Clause 55, wherein simulating various color perception tasks comprises presenting tasks under luminosities ranging from 10 lux to 100,000 lux.
Clause 57. The method of Clause 55 or 56, wherein simulating various backgrounds comprises presenting solid colors, gradients, and real-world scenes including urban landscapes and natural settings.
Clause 58. The method of any of Clauses 55-57, further comprising presenting a sequence of color perception tasks, wherein the tasks progress from low luminosity to high luminosity conditions.
Clause 59. The method of Clause 58, wherein the sequence of color perception tasks includes transitioning between different backgrounds to assess adaptability in color perception.
Clause 60. The method of any of Clauses 55-59, wherein evaluating the tracked data comprises: assessing gaze direction, fixation points, and response times; measuring color discrimination accuracy; calculating reaction times across varying luminosities; and determining error rates under specific conditions.
Clause 61. The method of Clause 60, wherein assessing gaze direction comprises identifying frequent shifts in gaze that may indicate difficulty in maintaining focus on certain colors under specific conditions.
Clause 62. The method of Clause 61, wherein assessing fixation points comprises identifying longer fixation durations on particular colors or backgrounds that may suggest challenges in distinguishing these colors from their surroundings.
Clause 63. The method of any of Clauses 55-62, further comprising: generating a color perception profile based on the evaluated tracked data; and providing recommendations for improving color perception in challenging scenarios.
Clause 64. The method of Clause 63, wherein the recommendations include suggestions for environmental modifications to enhance color perception.
Clause 65. The method of any of Clauses 55-64, further comprising compiling a comprehensive report including detailed color perception capabilities, identified deficiencies, and recommendations for improving color perception.
Clause 66. The method of any of Clauses 55-64, wherein evaluating the tracked data includes assessing color perception separately for different lighting conditions and background complexities.
Clause 67. The method of any of Clauses 55-66, further comprising calibrating the system using a control group with known color perception profiles to establish baseline metrics.
Clause 68. The method of any of Clauses 55-67, wherein simulating various color perception tasks comprises: presenting a sequence of different scenarios, each scenario lasting for a predetermined duration; progressively increasing the complexity of color distinctions throughout the sequence; and incorporating transitions between different luminosities and backgrounds to assess the user's adaptability to changing conditions.
Clause 69. The method of any of Clauses 55-68, further comprising: establishing baseline performance metrics by comparing the user's color perception data with profiles of individuals with normal color vision; identifying potential color perception deficiencies based on deviations from the established baseline; and providing recommendations for further color vision evaluation if significant deviations are detected.
Clause 70. The method of any of Clauses 55-69, wherein the eye-tracking sensors comprise infrared cameras with high-frequency tracking of at least 120 Hz, millisecond latency, and sub-millimeter precision.
Clause 71. The method of any of Clauses 55-70, wherein evaluating the tracked data comprises mapping the user's gaze direction, fixation points, and response times to their color perception accuracy and adaptability.
Clause 72. The method of any of Clauses 55-71, further comprising: simulating the effect of different environmental modifications in the virtual environment; allowing the user to experience and compare the simulated modifications in real-time; receiving user feedback on the simulated modifications; and providing final recommendations that balance objective color perception data with subjective user preferences.
Clause 73. A method of implementing a virtual reality (VR) system for evaluating color perception, comprising: at an electronic device including a head-mounted display (HMD) and eye-tracking sensors: generating a VR user interface corresponding to a three-dimensional virtual environment; rendering the VR user interface on the head-mounted display; simulating various color wavelength tasks in the VR user interface; and while simulating the color wavelength tasks, in real time: continuously tracking, using the eye-tracking sensors, user responses to the simulated tasks; and evaluating the tracked data for color wavelength sensitivity performance.
Clause 74. The method of Clause 73, wherein simulating various color wavelength tasks comprises presenting tasks involving primary colors (red, green, blue) in their purest form.
Clause 75. The method of Clause 73 or 74, wherein simulating various color wavelength tasks comprises introducing variations within primary colors, testing shades and intensities that differ slightly from the base wavelength.
Clause 76. The method of any of Clauses 73-75, further comprising presenting a sequence of color wavelength tasks, wherein initial exposures last 2-3 seconds per color, followed by longer exposures of 10-15 seconds.
Clause 77. The method of Clause 76, wherein the sequence of color wavelength tasks includes repeating tasks with increasing complexity to ensure consistent responses.
Clause 78. The method of any of Clauses 73-77, wherein evaluating the tracked data comprises: assessing gaze direction, fixation duration, and response accuracy; measuring color discrimination ability; calculating reaction times across different wavelengths; and determining error rates for color identification tasks.
Clause 79. The method of Clause 78, wherein assessing gaze direction comprises identifying which parts of the visual field the user focuses on when exposed to specific wavelengths.
Clause 80. The method of Clause 79, wherein assessing fixation duration comprises identifying longer fixation times on particular wavelengths that may indicate increased sensitivity or difficulty distinguishing the color.
Clause 81. The method of any of Clauses 73-80, further comprising: generating a color sensitivity profile based on the evaluated tracked data; and providing recommendations for specialized eyewear prescriptions.
Clause 82. The method of Clause 73-81, wherein the recommendations include suggestions for lenses designed to filter out problematic wavelengths.
Clause 83. The method of any of Clauses 73-82, further comprising compiling a comprehensive report including detailed sensitivity to specific color wavelengths, recommendations for specialized eyewear, and performance data.
Clause 84. The method of any of Clauses 73-83, wherein evaluating the tracked data includes assessing color wavelength sensitivity separately for different shades and intensities of primary colors.
Clause 85. The method of any of Clauses 73-84, further comprising calibrating the system using a control group with established color sensitivity profiles to establish baseline performance metrics.
Clause 86. The method of any of Clauses 73-85, wherein simulating various color wavelength tasks comprises: presenting a sequence of different color scenarios, each scenario lasting for a predetermined duration; progressively increasing the complexity of color wavelength distinctions throughout the sequence; and incorporating transitions between different color wavelengths to assess the user's adaptability to changing conditions.
Clause 87. The method of any of Clauses 73-86, further comprising: establishing baseline performance metrics by comparing the user's color wavelength sensitivity data with profiles of individuals with normal color vision; identifying potential color wavelength sensitivity issues based on deviations from the established baseline; and providing recommendations for further color vision evaluation if significant deviations are detected.
Clause 88. The method of any of Clauses 73-87, wherein the eye-tracking sensors comprise high-precision sensors capable of tracking micro-movements with an accuracy of 0.1 degrees in gaze direction and a latency under 5 milliseconds.
Clause 89. The method of any of Clauses 73-88, wherein evaluating the tracked data comprises mapping the user's gaze direction, fixation duration, and response accuracy to specific color wavelengths.
Clause 90. The method of any of Clauses 73-89, further comprising: simulating the effect of different specialized eyewear prescriptions in the virtual environment; allowing the user to experience and compare the simulated prescriptions in real-time; receiving user feedback on the simulated prescriptions; and providing final recommendations that balance objective color wavelength sensitivity data with subjective user preferences.
Clause 91. A method of implementing a virtual reality (VR) system for testing and recommending adaptive eyewear for color blindness, comprising: generating a VR user interface corresponding to a three-dimensional virtual environment; rendering the VR user interface on the head-mounted display; simulating various real-world scenarios in the VR user interface; and while simulating the real-world scenarios, in real time: continuously tracking, using the eye-tracking sensors, user responses to the simulated scenarios; and evaluating the tracked data for color perception performance.
Clause 92. The method of Clause 91, wherein simulating various real-world scenarios comprises presenting tasks involving color-critical situations such as distinguishing traffic lights or selecting ripe fruits.
Clause 93. The method of Clause 91 or 92, wherein simulating various real-world scenarios comprises incorporating dynamic changes in lighting and context that affect color perception in daily activities.
Clause 94. The method of any of Clauses 91-93, further comprising presenting a sequence of real-world simulations, wherein each simulation tests different aspects of color perception relevant to daily life.
Clause 95. The method of Clause 94, wherein the sequence of real-world simulations includes progressively challenging scenarios to assess the full range of the user's color perception capabilities.
Clause 96. The method of any of Clauses 91-95, wherein evaluating the tracked data comprises: assessing color identification accuracy; measuring reaction times to color-based cues; evaluating performance in color-dependent tasks; and determining error rates for color-critical decisions.
Clause 97. The method of Clause 96, wherein assessing color identification accuracy comprises comparing the user's color identifications with known color values in the simulated scenarios.
Clause 98. The method of Clause 96, wherein evaluating performance in color-dependent tasks comprises analyzing the user's ability to complete tasks that require accurate color perception.
Clause 99. The method of any of Clauses 91-97, further comprising: generating a color vision profile based on the evaluated tracked data; and providing personalized recommendations for adaptive eyewear.
Clause 100. The method of Clause 99, wherein the recommendations include suggestions for specific tints or filters that enhance the user's color perception in identified challenging scenarios.
Clause 101. The method of any of Clauses 91-100, further comprising compiling a comprehensive report including detailed color vision capabilities, personalized adaptive eyewear recommendations, and performance metrics.
Clause 102. The method of any of Clauses 91-101, wherein evaluating the tracked data includes assessing color perception separately for different types of real-world scenarios and lighting conditions.
Clause 103. The method of any of Clauses 91-102, further comprising calibrating the system using a control group with known color vision profiles to establish baseline performance metrics.
Clause 104. The method of any of Clauses 91-103, wherein simulating various real-world scenarios comprises: presenting a sequence of different color-critical situations, each situation lasting for a predetermined duration; progressively increasing the complexity of color perception challenges throughout the sequence; and incorporating transitions between different lighting conditions to assess the user's adaptability to changing environments.
Clause 105. The method of any of Clauses 91-104, further comprising: establishing baseline performance metrics by comparing the user's color perception data with profiles of individuals with normal color vision; identifying specific types and degrees of color blindness based on deviations from the established baseline; and providing recommendations for further medical evaluation if significant color vision deficiencies are detected.
Clause 106. The method of any of Clauses 91-105, wherein the eye-tracking sensors comprise high-precision sensors capable of tracking micro-movements and pupil dilation in response to different color stimuli.
Clause 107. The method of any of Clauses 91-106, wherein evaluating the tracked data comprises mapping the user's color identification accuracy, reaction times, and task performance to specific types of color blindness.
Clause 108. The method of any of Clauses 91-107, further comprising: simulating the effect of different adaptive eyewear options in the virtual environment; allowing the user to experience and compare the simulated adaptive eyewear in real-time across various scenarios; receiving user feedback on the simulated adaptive eyewear options; and providing final recommendations that balance objective color perception data with subjective user preferences and comfort.
Clause 109. A system for implementing a virtual eye test, comprising: a head-mounted display including a display and one or more cameras; one or more processors; and memory storing one or more programs configured to be executed by the one or more processors, the one or more programs including instructions for performing the method of any of Clauses 1-108.

In some embodiments, any of the above clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

### Further Considerations

As used herein, the word “module” refers to logic embodied in hardware or firmware, or to a collection of software instructions, possibly having entry and exit points, written in a programming language, such as, for example C++. A software module may be compiled and linked into an executable program, installed in a dynamic link library, or may be written in an interpretive language such as BASIC. It will be appreciated that software modules may be callable from other modules or from themselves, and/or may be invoked in response to detected events or interrupts. Software instructions may be embedded in firmware, such as an EPROM or EEPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware.

It is contemplated that the modules may be integrated into a fewer number of modules. One module may also be separated into multiple modules. The described modules may be implemented as hardware, software, firmware or any combination thereof. Additionally, the described modules may reside at different locations connected through a wired or wireless network, or the Internet.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise”as “comprise”is interpreted when employed as a transitional word in a claim.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items.

As used herein, the term “comprising” indicates the presence of the specified integer(s), but allows for the possibility of other integers, unspecified. This term does not imply any particular proportion of the specified integers. Variations of the word “comprising,”such as “comprise”and “comprises,”have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope. In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. A method of implementing a virtual reality (VR) system for evaluating color perception, comprising:
at an electronic device including a head-mounted display (HMD) and eye-tracking sensors:
generating a VR user interface corresponding to a three-dimensional virtual environment;
rendering the VR user interface on the head-mounted display;
simulating various color wavelength tasks in the VR user interface; and
while simulating the color wavelength tasks, in real time:
continuously tracking, using the eye-tracking sensors, user responses to the simulated tasks; and
evaluating the tracked data for color wavelength sensitivity performance.

2. The method of claim 1, wherein simulating various color wavelength tasks comprises presenting tasks involving primary colors (red, green, blue) in their purest form.

3. The method of claim 1, wherein simulating various color wavelength tasks comprises introducing variations within primary colors, testing shades and intensities that differ slightly from the base wavelength.

4. The method of claim 1, wherein simulating various color wavelength tasks comprises:
presenting a sequence of different color scenarios, each scenario lasting for a predetermined duration;
progressively increasing the complexity of color wavelength distinctions throughout the sequence; and
incorporating transitions between different color wavelengths to assess the user's adaptability to changing conditions.

5. The method of claim 1, wherein the eye-tracking sensors comprise high-precision sensors capable of tracking micro-movements with an accuracy of 0.1 degrees in gaze direction and a latency under 5 milliseconds.

6. The method of claim 1, wherein evaluating the tracked data comprises:
assessing gaze direction, fixation duration, and response accuracy;
measuring color discrimination ability;
calculating reaction times across different wavelengths; and
determining error rates for color identification tasks.

7. The method of claim 6, wherein assessing gaze direction comprises identifying parts of the visual field the user focuses on when exposed to specific wavelengths.

8. The method of claim 6, wherein assessing fixation duration comprises identifying longer fixation times on particular wavelengths that may indicate increased sensitivity or difficulty distinguishing the color.

9. The method of claim 1, wherein evaluating the tracked data comprises assessing color wavelength sensitivity separately for different shades and intensities of primary colors.

10. The method of claim 1, wherein evaluating the tracked data comprises mapping the user's gaze direction, fixation duration, and response accuracy to specific color wavelengths.

11. The method of claim 1, further comprising presenting a sequence of color wavelength tasks, wherein initial exposures last 2-3 seconds per color, followed by longer exposures of 10-15 seconds.

12. The method of claim 11, wherein the sequence of color wavelength tasks comprises repeating tasks with increasing complexity to ensure consistent responses.

13. The method of claim 1, further comprising:
generating a color sensitivity profile based on the evaluated tracked data; and
providing recommendations for specialized eyewear prescriptions.

14. The method of claim 13, wherein the recommendations comprise suggestions for lenses designed to filter out problematic wavelengths.

15. The method of claim 1, further comprising compiling a comprehensive report including detailed sensitivity to specific color wavelengths, recommendations for specialized eyewear, and performance data.

16. The method of claim 1, further comprising calibrating the system using a control group with established color sensitivity profiles to establish baseline performance metrics.

17. The method of claim 1, further comprising:
establishing baseline performance metrics by comparing the user's color wavelength sensitivity data with profiles of individuals with normal color vision;
identifying potential color wavelength sensitivity issues based on deviations from the established baseline; and
providing recommendations for further color vision evaluation if significant deviations are detected.

18. The method of claim 1, further comprising:
simulating the effect of different specialized eyewear prescriptions in the virtual environment;
allowing the user to experience and compare the simulated prescriptions in real-time;
receiving user feedback on the simulated prescriptions; and
providing final recommendations that balance objective color wavelength sensitivity data with subjective user preferences.

19. A system for recommending lens tints through an interactive vision sensitivity test, comprising:
a head-mounted display;
eye-tracking sensors;
one or more processors; and
memory storing one or more programs configured to be executed by the one or
more processors, the one or more programs including instructions for:
generating a VR user interface corresponding to a three-dimensional virtual environment;
rendering the VR user interface on the head-mounted display;
simulating various color wavelength tasks in the VR user interface; and
while simulating the color wavelength tasks, in real time:
continuously tracking, using the eye-tracking sensors, user responses to the simulated tasks; and
evaluating the tracked data for color wavelength sensitivity performance.

20. A non-transitory computer-readable storage medium storing one or more programs configured to be executed by one or more processors of an electronic device with a head-mounted display and eye-tracking sensors, the one or more programs including instructions for:
generating a VR user interface corresponding to a three-dimensional virtual environment;
rendering the VR user interface on the head-mounted display;
simulating various color wavelength tasks in the VR user interface; and
while simulating the color wavelength tasks, in real time:
continuously tracking, using the eye-tracking sensors, user responses to the simulated tasks; and
evaluating the tracked data for color wavelength sensitivity performance.
